(19) United States
(12) Patent Application Publication
(10) Pub. No.: US 2023/0026402 A1
(43) Pub. Date: Jan. 26, 2023

(54) MICROBIOTA BASED THERAPIES TO PROMOTE MENTAL HEALTH

(71) Applicant: Cornell University, Ithaca, NY (US)

(72) Inventor: David ARTIS, New York, NY (US)

(21) Appl. No.: 17/770,740

(22) PCT Filed: Oct. 22, 2020
(86) PCT No.: PCT/US2020/056918

§ 371 Date: Apr. 21, 2022

Related U.S. Application Data
(60) Provisional application No. 62/924,548, filed on Oct. 22, 2019.

(51) Int. Cl. A61K 35/742; A61K 31/255; A61K 31/404; A61K 35/741; A61P 25/22

(52) U.S. Cl. CPC A61K 35/742; A61K 31/255; A61K 31/404; A61K 35/741; A61P 25/22

(57) ABSTRACT

Provided are compositions and methods for treatment of neurological and behavioral disorders. Compositions comprise two or more of phenyl sulfate, pyrocatechol sulfate, 3-(3-sulfooxyphenyl)propanoic acid, and indoxyl sulfate, and optionally bacteria which can increase the concentrations of phenyl sulfate, pyrocatechol sulfate, 3-(3-sulfooxyphenyl)propanoic acid, and/or indoxyl sulfate. Methods comprise administering to an individual who is afflicted with a neurological or behavioral disorder a composition comprising two or more of phenyl sulfate, pyrocatechol sulfate, 3-(3-sulfooxyphenyl)propanoic acid, and indoxyl sulfate, and optionally, bacteria that can produce phenyl sulfate, pyrocatechol sulfate, 3-(3-sulfooxyphenyl)propanoic acid, and indoxyl sulfate.

### CROSS-REFERENCE TO RELATED APPLICATIONS

This application claims priority to U.S. Provisional patent application No. 62/924,548, filed on Oct. 22, 2019, the entire disclosure of which is incorporated herein by reference.

### BACKGROUND OF THE DISCLOSURE

In the classical fear conditioning paradigm, extinction learning occurs when repeated cue presentations are no longer paired with an unconditioned stimulus (such as a foot shock) and the organism learns to modify its behavior accordingly. Deficits in extinction learning after an environmental threat has passed have been implicated in multiple neuropsychiatric disorders, including post-traumatic stress disorder and other anxiety disorders (VanElzakker et al., 2014, Neurobiol Learn Mem 113, 3-18). Clinical and epidemiological studies have reported correlations between changes in the microbiota and other neuropsychiatric disorders ((Krajmalnik-Brown et al., 2015, Microb Ecol Health Dis 26, 26914; Mielcarz and Kasper, 2015, Curr Treat Options Neurol 17, 344; Zheng et al., 2016, Mol Psychiatry 21, 786-796). Animal studies indicate that the absence or modification of the intestinal microbiota affects neurogenesis (Mohle et al., 2016, Cell Rep 15, 1945-1956), cortical myelination (Hoban et al., 2016, Transl Psychiatry 6, e774), blood-brain barrier function (Braniste et al., 2014, Sci Transl Med 6, 263ra158), and microglia maturation (Erny et al., 2015, Nat Neurosci 18, 965-977), as well as social behavior, stress-related responses and fear learning (Vuong et al., 2017, Annu Rev Neurosci 40, 21-49; Hoban et al., 2016, Transl Psychiatry 6, e774). However, there are conflicting reports on how the microbiota influence behavior ((Arentsen et al., 2015, Microb Ecol Health Dis 26, 29719; Desbonnet et al., 2014, Mol Psychiatry 19, 146-148; Hoban et al., 2016, Transl Psychiatry 6, e774; Lu et al., 2018, PLoS One 13, e0201829) and the mechanisms through which the microbiota regulate associative learning and its neurobiological substrates remain unclear. As such there is a need to better define the co-evolved relationship between the microbiota and the nervous system in an effort to treat mammalian behavioral or learning disorders.

### SUMMARY OF THE DISCLOSURE

This disclosure provides compositions and methods for treatment of behavioral and neurological diseased conditions. The disclosure is based at least on the findings that in manipulation of the microbiota results in significant deficits in fear extinction learning. Single nucleus RNA-seq of the medial prefrontal cortex of the brain in either antibiotic-treated or germ-free animal models revealed significant alterations in gene expression in multiple cell types including excitatory neurons. Transcranial two-photon imaging following deliberate manipulation of the microbiota demonstrated that extinction learning deficits were associated with defective learning-related remodeling of postsynaptic dendritic spines and reduced activity in cue-encoding neurons in the medial prefrontal cortex. In addition to effects of manipulating the microbiota on behavior in adult mice, selective re-establishment of the microbiota revealed a limited neonatal developmental window in which microbiota-derived signals can restore normal extinction learning in adulthood. Unbiased metabolomic analysis identified four metabolites that were significantly downregulated in antibiotic-treated and germ-free animals, indicating microbiota-derived compounds may affect brain function and behavior. Based on these data, the present disclosure provides metabolite and/or probiotic compositions and methods for treatment of behavioral or neuropsychiatric disorders.

In an aspect, this disclosure provides compositions comprising or consisting essentially of i) one or more of phenyl sulfate, pyrocatechol sulfate, 3-(3-sulfooxyphenyl)propanoic acid, and indoxyl sulfate, and/or ii) bacteria that produce one or more of phenyl sulfate, pyrocatechol sulfate, 3-(3-sulfooxyphenyl)propanoic acid, and indoxyl sulfate. In an embodiment, this disclosure provides compositions comprising or consisting essentially of i) one or more of phenyl sulfate, pyrocatechol sulfate, 3-(3-sulfooxyphenyl)propanoic acid, and indoxyl sulfate, and/or ii) bacteria from the Clostridium or Bacteroides species, which are not pathogenic bacteria.

In an aspect, this disclosure provides methods for promoting the neurological health, psychological health or brain health of an individual comprising contacting the individual with or administering to the individual in need of treatment a composition comprising, consisting essentially of, or consisting of one or more i) bacteria which produce one or more of phenyl sulfate, pyrocatechol sulfate, 3-(3-sulfooxyphenyl)propanoic acid, and indoxyl sulfate, or ii) one or more of phenyl sulfate, pyrocatechol sulfate, 3-(3-sulfooxyphenyl)propanoic acid, and indoxyl sulfate, or a compositing a prodrug that can be converted to one or more of phenyl sulfate, pyrocatechol sulfate, 3-(3-sulfooxyphenyl)propanoic acid, and indoxyl sulfate, or a combination of i) and ii). In an embodiment, the method comprises or consists essentially of administering to an individual in need of treatment a composition comprising, consisting essentially or consisting of one or more of phenyl sulfate, pyrocatechol sulfate, 3-(3-sulfooxyphenyl)propanoic acid, and indoxyl sulfate, and/or one or more non-pathogenic Clostridium or Bacteroides species. The neurological condition may be anxiety disorder, including panic disorders and post-traumatic stress disorder.

### BRIEF DESCRIPTION OF THE FIGURES

FIG. 1. ABX and GF mice are less prone to fear extinction. a, Acquisition of fear conditioning quantified by percent time spent freezing during fear conditioning (FC) tone 3. Percent time spent freezing was calculated by dividing the amount of time spent freezing during the tone presentation by the duration of the tone (30 s). b, Fear extinction in Ctrl and ABX mice over the course of 3 days/sessions. c, Fear extinction of Ctrl and ABX mice after three days/sessions displayed by differential extinction indices [(Session 1 Tone 1, S1T1)—(Session 3 Tone 5, S3T5)]. Data in a-c are pooled from two independent experiments, n=30/group. Data are mean±SEM. Unpaired two-sided t tests were used in a and c. The area under the curve (AUC) was calculated for each mouse within each group in b, followed by unpaired two-sided t test between groups. P values are indicated on the figure. d, Fear extinction of Ctrl and ABX mice in the single-session 30-tone fear extinction assay. Data are pooled from two independent experiments, n=12/group. Data are mean±SEM. The area under the curve (AUC) was calculated for each mouse within each group followed by unpaired two-sided t test between groups. P value is indicated on the figure. e, Fear extinction of Ctrl and GF mice in the single-session 30-tone fear extinction assay. Data are pooled from two independent experiments, n=12/group. Data are mean±SEM. The area under the curve (AUC) was calculated for each mouse within each group followed by unpaired two-sided t test between groups. P value is indicated on the figure. f, Principle component analysis (PCA) of genome-wide transcriptional profiles of mouse mPFC post fear extinction. n=4/group. PERMANOVA test was used: F=5.00, Df=1, P=0.027. g, Volcano plot of differential expression between Ctrl (negative log₂ (fold change (FC))) and ABX (positive log₂ (FC)) groups in f. Differentially expressed genes (DESeq2 Wald test, False Discovery Rate (FDR)<0.1) are shown in red. h, Heatmap of gene-expression profiles showing the top 50 most significantly (according to DESeq2 Wald test P value) downregulated (left) and upregulated (right) genes in ABX versus Ctrl in f. Lowly-expressed genes with mean normalized counts in the bottom 20th percentile were excluded. i,j, Search tool for recurring instances of neighboring genes (STRING) network visualization of the top 50 most significantly (according to P value) downregulated (i) and upregulated (j) genes in ABX versus Ctrl in f. Network nodes represent proteins and are filled with their 3D structures unless the structure is unknown. Edges represent protein-protein associations. Disconnected nodes were excluded. P value is indicated on the figure. k, Significantly enriched KEGG pathways based on all differentially expressed genes in g. l-o, Immunofluorescence staining of DAPI (blue) and c-Fos (red) (l,n) and the density of c-Fos⁺ neurons (m,o) in the BLA (l,m) or IL (n,o) of Ctrl and ABX mice 90 min after classical fear extinction session 3. Data are representative of two independent experiments. n=4/group. Data are mean±SEM. Unpaired two-sided t tests were used. P values are indicated on the figure. BLA, basolateral amygdala; PL, prelimbic cortex; IL, infralimbic cortex. Scale bar, 200 μm.

FIG. 2. Excitatory neurons and microglia are affected in ABX mice. a, Uniform manifold approximation and projection (UMAP) of single nucleus profiles (dots) colored by cell type. Clusters are labeled with post facto annotation based on known marker genes (Data FIG. 10); Ambiguous clusters expressing multiple canonical markers across cell types are annotated with both, e.g. exPFC/Astrocyte, and is likely due to doublets. n=2/group. b, Scatter plot of the number of significant DEGs vs. the number of nuclei per cluster. Dots are labeled by their corresponding cluster color in a. c,d, Differential expression of ABX vs. Ctrl (log₂(Fold change), x axis) in a in excitatory neuron subset 1 (exPFC1) (c) or microglia (d) and the associated significance (−log₁₀ (P-value) with Bonferroni correction, y axis; linear regression, Methods). Blue: genes significantly differentially expressed (P<10⁻⁷ with Bonferroni correction). e, Gene ontology (GO) terms significantly enriched among the differentially expressed genes in d.

FIG. 3. Defective extinction learning-related dendritic spine formation in ABX mice. a, Diagrammatic representation of a coronal section of mPFC showing the imaging site (cyan bar). FrA, frontal association cortex. b, Example images of neuronal dendritic branch segments at two consecutive imaging time points. Red (top) and blue (bottom) arrows indicate spine elimination and formation, respectively. Scale bar=5 μm. F, filopodia. c, Timeline of transcranial two-photon spine imaging combined with the cranial window implantation surgery, the antibiotic treatment and the fear conditioning and extinction assay. Mice were on antibiotics since day 0. d,e, Percentage of spine elimination (d) and formation (e) at baseline, during fear conditioning and during fear extinction, respectively. f, g, Ratio of ABX to Ctrl on spine elimination rate (f) and spine formation rate (g). Data in d-g are pooled from three independent experiments, n=5/group. Center line, median; box, 25^{th} and 75^{th} percentiles; whiskers, mean to max. Unpaired two-sided t tests were used in d and e. P values are as follows: i=0.0010, ii=4.38E-05, iii=0.0002. h,i, Immunofluorescence staining (h) and the area (1) of synaptophysin in the mPFC of Ctrl and GF mice. j,k, Immunofluorescence staining (j) and the area (k) of PSD-95 in the mPFC of Ctrl and GF mice. Data are pooled from two independent experiments. n=6/group. Each symbol represents one region of interest (ROI). 5 ROIs per mouse. Data are mean±SEM. Unpaired two-sided t tests were used. P values are indicated on the figure. Scale bar, 10 μm.

FIG. 4. Defective ensemble calcium dynamics in the mPFC of ABX mice. a, Example false-color image of the 7 min 30 sec long time series (mean projection over time) of GCaMP6s-expressing neurons in mPFC. Scale bar=50 μm. b, Segmentation of the neurons in (a). c, Neuronal activity time series (ΔF/F) extracted from the 3 example neurons outlined in (b). d, Population activity trace (mean ΔF/F±SEM) for neurons exhibiting decreased activity during tone presentations in fear extinction session 3. e, Mean activity ΔF/F) during the baseline (pre-tone) period, during tone presentations (“tone-on”), and during the inter-tone periods (“tone-off”) for the neuronal population depicted in (d), showing a significant decrease in activity (repeated measures ANOVA: main effect of time: F (10,1600)=3.138, P=0.007) but no significant difference between groups (group-by-time interaction: F (10,1600)=2.736, P=0.1280). NS=not significant (baseline: P=0.285; tone-on: P=0.595; tone-off: P=0.578). Center line, median; box, 25^{th} and 75^{th} percentiles; whiskers, mean to max. f, Population activity trace (mean ΔF/F±SEM) for neurons exhibiting increased activity during tone presentations in fear extinction session 3. g, Mean activity ΔF/F) during each task epoch (baseline, tone-on, tone-off) for the neuronal population depicted in (f), showing a significant increase in activity (repeated measures ANOVA: main effect of time: F (10,1770)=4.945, P<0.0001) and a significant group-by-time interaction (F (10,1770)=3.806, P=0.0008). *=significant group difference in post-hoc contrast, P=0.013. NS=not significant (baseline: P=0.128; tone off: P=0.601). Center line, median; box, 25^{th} and 75^{th} percentiles; whiskers, mean to max. h, Raster plot of neuronal activity for cells that encoded the timing of tones by increasing and decreasing activity in response to tone onset and offset, respectively. Each row indicates one neuron. i, Population activity trace (mean ΔF/F±SEM) for neurons depicted in (h), timelocked to tone onset and averaged across tones. Repeated measures ANOVA: main effect of time: F (179,8234)=7.033, P<0.0001; group by time interaction: F (179,8234)=2.749, P=0.0093. Data in d-i are based on 1,204 total cells pooled from three independent experiments, from n=7 Ctrl mice and n=8 ABX mice.

FIG. 5. Extinction learning deficits in GF mice are associated with alterations in microbiota-derived metabolites. a, Fear extinction of Ctrl, GF and gnotobiotic mice colonized by SFB, Clostridia, Enterobacter or ASF bacterium (−a) in the single-session 30-tone fear extinction assay. Data are pooled from three independent experiments. Ctrl n=9, GF n=13, SFB n=9, Clostridia n=12, Enterobacter n=7, ASF n=9. Data are mean±SEM. The area under the curve (AUC) was calculated for each mouse within each group followed by one-way ANOVA with Tukey's multiple comparisons test. F (5, 53)=7.046. P=4.10E-05. Adjusted P values are as follows: i=6.34E-05, ii=0.0002, iii=0.0042, iv=0.0010, v=0.0189. b, Fear extinction of Ctrl, GF and ex-GF_adult mice in the single-session 30-tone fear extinction assay. Data are pooled from three independent experiments, n=18/group. Data are mean±SEM. The area under the curve (AUC) was calculated for each mouse within each group followed by one-way ANOVA with Tukey's multiple comparisons test. F (2, 51)=11.92. P=5.66E-05. Adjusted P values are as follows: i=0.0002, ii=0.0005. c, Fear extinction of Ctrl, GF and ex-GF_weaning mice in the single-session 30-tone fear extinction assay. Data are pooled from two independent experiments, n=12/group. Data are mean±SEM. The area under the curve (AUC) was calculated for each mouse within each group followed by one-way ANOVA with Tukey's multiple comparisons test. F (2, 33)=12.64. P=8.40E-05. Adjusted P values are as follows: i=0.0016, ii=0.0001. d, Fear extinction of Ctrl, GF and ex-GF_fostered mice in the single-session 30-tone fear extinction assay. Data are pooled from three independent experiments. Ctrl_fostered n=10, GF n=11, GF_fostered n=12. Data are mean±SEM. The area under the curve (AUC) was calculated for each mouse within each group followed by one-way ANOVA with Tukey's multiple comparisons test. F (2, 30)=5.131. P=0.0121. Adjusted P values are as follows: i=0.0228, ii=0.0273. e,f, Relative abundances of four compounds in cerebrospinal fluid (CSF) (e) and serum (f) samples from Ctrl_fostered, GF and GF_fostered mice as determined by LC-MS. n=3/group. Data are mean±SEM. Unpaired two-sided t tests were used. P values are as follows: i=9.54E-05, ii=0.0102, iii=0.0036, iv=0.0044, v=0.0018, vi=0.0011, vii=0.1493, viii=0.0331 in (e); i=0.0017, ii=0.0002, iii=0.0005, iv=0.0128, v=0.0014, vi=0.0003, vii=0.0020, viii=0.0014 in (f).

FIG. 6. Antibiotic treatment results in bacterial community restructuring. a-c, Food intake (a), water intake (b) and weight gain (c) of the mice measured by the Promethion Metabolic Cage System. Antibiotic treatment was started 2 weeks prior to the experiment and continued for the duration of the experiment. For food (a) and water intake (b), the mice were acclimated to the system for the first 4 days followed by 1 day of data collection. Body mass (c) of the mice were measured at the beginning of (Start) and the end (End) of the 5-day experiment. n=4/group. Data are mean±SEM. Total, full day. Light/Dark, 12-hour light/dark cycle. d, 16S rDNA gene copies as quantified by real-time RT-PCR from stool pellets collected from control (Ctrl) or ABX mice. Data are pooled from two independent experiments. n=7/group. Data are mean±SEM. Unpaired two-sided t tests were used. e-g, Principal coordinates analysis (PCoA) (e), alpha-diversity Shannon index (f) and taxonomic classification (g) of 16S rDNA in stool pellets collected from Ctrl or ABX mice. Ctrl n=4, ABX n=5. For PCoA plot PERMANOVA: F=33.579, Df=1, P=0.00804. For phylogenetic classification ‘f_’, ‘g_’, ‘uncl_c_’, ‘uncl_d_’ and ‘uncl_o_’ stand for ‘family_’, ‘genus_’, ‘unclassified_class_’, ‘unclassified_domain_’ and ‘unclassified_order_’, respectively. ‘uncl_d_Bacteria’ matches exactly to mitochondria or chloroplasts, most likely from the food. Data are mean±SEM in f.

FIG. 7. Antibiotic-treated mice retain deficits in extinction learning after vagotomy. Fear extinction in Ctrl Sham, ABX_Sham and ABX_Vx mice over the course of 3 days/sessions. Ctrl Sham n=10, ABX_Sham n=10, ABX_Vx n=12. Data are mean±SEM. The area under the curve (AUC) was calculated for each mouse within each group, followed by unpaired two-sided t test between groups. P values are as follows: i=2.57E-07, ii=9.21E-08. Vx, vagotomized.

FIG. 8. Comparable percentages and numbers of CD45^{high} leukocytes in the brain of Ctrl and ABX/GF mice. a, Gating strategy of T cells, B cells, dendritic cells (DCs) and macrophages (Mφ) in the brain. b, Population frequencies and numbers of brain-resident CD45^{high} leukocytes in Ctrl and ABX mice. c-d, Population frequencies of CD4⁺ T cells, CD8⁺ T cells, CD19⁺ B cells (c), CD11c⁺ DCs and F4/80⁺ macrophages (d) gated on brain-resident CD45^{high} leukocytes in Ctrl and ABX mice. e, Population frequencies and numbers of brain-resident CD45^{high} leukocytes in Ctrl and GF mice. f-g, Population frequencies of CD4⁺ T cells, CD8⁺ T cells, CD19⁺ B cells (f), CD11c⁺ DCs and F4/80⁺ macrophages (g) gated on brain-resident CD45^{high} leukocytes in Ctrl and GF mice. h, Gating strategy of total myeloid cells and Ly6C^{hi} monocytes in the brain. i,j, Population frequencies of total myeloid cells and Ly6C^{hi} monocytes gated on brain-resident CD45^{high} leukocytes in Ctrl and ABX (i) or GF (j) mice. Data in b, c, g and j are representative of three independent experiments. n=4/group. Data in d and i are pooled from two independent experiments. n=8/group. Data in e and f are pooled from two independent experiments. n=6/group. Data are mean±SEM. Unpaired two-sided t tests were used. P values are indicated on the figure. k, Fear extinction of Ctrl, GF and Rag1^{-/-} mice in the single-session 30-tone fear extinction assay. Data are pooled from two independent experiments. Ctrl n=18, GF n=16, Rag1^{-/-} n=18. Data are mean±SEM. The area under the curve (AUC) was calculated for each mouse within each group followed by one-way ANOVA with Tukey's multiple comparisons test. F (2, 49)=8.558. P=0.0006. Adjusted P values are as follows: i=0.0343, ii=0.0004. l, Fear extinction of SPF-Rag1^{-/-} and GF-Rag1 mice in the single-session 30-tone fear extinction assay. n=7/group. Data are mean±SEM. The area under the curve (AUC) was calculated for each mouse within each group followed by unpaired two-sided t test between groups. P value is indicated on the figure.

FIG. 9. Comparable transcriptomes of mPFCs dissected from Ctrl and ABX mice in the absence of fear conditioning and extinction. a, Principle component analysis (PCA) of genome-wide transcriptional profiles of mouse mPFC in the absence of fear conditioning and extinction. Ctrl n=3, ABX n=4. PERMANOVA test was used: F=2.52, Df=1, P=0.17. b, Volcano plot of differential expression between Ctrl (negative log₂ (fold change (FC))) and ABX (positive log₂ (FC)) groups. Differentially expressed genes (defined as False Discovery Rate (FDR)<0.1, DESeq2 Wald test) are shown in red. c-f, Immunofluorescence staining of c-Fos (red) (c,e) and the density of c-Fos⁺ neurons (d,f) in the BLA (c,d) or IL (e,f) of Ctrl and GF mice 90 min after classical fear extinction session 3. Data are pooled from two independent experiments. n=6/group. Data are mean±SEM. Unpaired two-sided t tests were used. P values are indicated on the figure. BLA, basolateral amygdala; PL, prelimbic cortex; IL, infralimbic cortex. Scale bar, 200 μm.

FIG. 10. Gene expression patterns of individual cell subsets in mPFC. Proportion of expressing cells (dot size) and mean normalized expression of representative marker genes (columns) associated with the cell clusters of FIG. 2a (rows). exPFC=glutamatergic excitatory neurons from the PFC, GABA=GABAergic interneurons, OPC=oligodendrocyte progenitor cells, MO=myelinating oligodendrocyte.

FIG. 11. Differential gene expression between Ctrl and ABX groups in individual clusters of mPFC. Differential expression of ABX vs. Ctrl (log₂(fold change), x axis) in each cluster in FIG. 2a and the associated significance (−log₁₀ (P-value), y axis; linear regression, Methods). Blue: genes significantly differentially expressed (P<10⁻⁷ with Bonferroni correction). exPFC=glutamatergic excitatory neurons from the PFC, GABA=GABAergic interneurons, OPC=oligodendrocyte progenitor cells, MO=myelinating oligodendrocyte.

FIG. 12. Differentially expressed genes of ABX vs. Ctrl mPFC samples shared by all excitatory neuronal subsets. Mean fold change in expression in excitatory neurons (columns) in FIG. 2a of genes (rows) that were significantly differentially expressed in at least 2 of these clusters, and with absolute (log 2fc)>=0.31 in at least one cluster.

FIG. 13. Differentially expressed genes of ABX vs. Ctrl mPFC samples shared by multiple cell types. Mean fold change in expression across all cell clusters (columns) in FIG. 2a of genes (rows) that were significantly differentially expressed in at least 4 clusters, and with absolute (log 2fc)>=0.31 in at least one cluster.

FIG. 14. Microglia in GF and ABX mice exhibit a developmentally immature phenotype. a, Population frequencies and numbers of microglia in Ctrl and GF mice. b, Representative flow cytometry histogram and mean fluorescence intensity (MFI) of F4/80 staining on microglia from Ctrl and GF mice. c, Representative flow cytometry plots and population frequencies of CSF1R⁺ microglia in Ctrl and GF mice. d, Representative flow cytometry histogram and MFI of CSF1R expression gated on CSF1R⁺ microglia from Ctrl and GF mice. Data in a-d are representative of three independent experiments. n=4/group. e, Population frequencies and numbers of microglia in Ctrl and ABX mice. f, Representative flow cytometry histogram and MFI of F4/80 staining on microglia from Ctrl and ABX mice. g, Representative flow cytometry plots and population frequencies of CSF1R⁺ microglia in Ctrl and ABX mice. h, Representative flow cytometry histogram and MFI of CSF1R expression gated on CSF1R⁺ microglia from Ctrl and ABX mice. Data in e are pooled from two independent experiments. n=8/group. Data in f-h are representative of two independent experiments. n=4/group. Data are mean±SEM. Unpaired two-sided t tests were used. P values are indicated on the figure.

FIG. 15. Downregulation of the metabolites in GF mice. a, ELISA quantification of plasma corticosterone in control and ABX mice. Data are pooled from three independent experiments. Control n=12. ABX n=11. b, ELISA quantification of plasma corticosterone in control and GF mice. Data are pooled from three independent experiments. Control n=12. GF n=11. Data are mean±SEM. c, Structures of phenyl sulfate, pyrocatechol sulfate, 3-(3-sulfooxyphenyl)propanoic acid and indoxyl sulfate. d, Relative abundances of phenyl sulfate, pyrocatechol sulfate, 3-(3-sulfooxyphenyl)propanoic acid and indoxyl sulfate in fecal samples from Ctrl_fostered, GF and ex-GF_fostered mice as determined by LC-MS. n=3/group. e, Relative abundances of phenyl sulfate, pyrocatechol sulfate, 3-(3-sulfooxyphenyl)propanoic acid and indoxyl sulfate in cerebrospinal fluid (CSF) samples from Ctrl and GF mice as determined by LC-MS. Data are representative of two independent experiments n=8/group. Data are mean±SEM. Unpaired two-sided t tests were used. P values are indicated on the figure. f, A schematic representation of the microbiota-gut-brain axis in fear extinction learning. Our data inform a model whereby alterations in the microbiota and their metabolites influence neuronal function and learning-related plasticity, which may be due to altered microglia-mediated synaptic pruning, and subsequently regulate fear extinction behavior.

FIG. 16. Illustration of method for dissociation of CNS cultures. Briefly, whole brain tissue is isolated from P1 C57Bl6 mice and dissociated into a single cell suspension using a combination of enzymatic and mechanical dissociation. For primary neuronal cultures, this cell suspension is plated directly only poly-L-lysine coated tissue culture plates or glass coverslips and grown in serum-free conditions (Neurobasal with B27 supplement). For microglia cultures, the single cell suspension is plated in glia growth media (DMEM+10% FCS with P/S/Q) and allowed to grow to confluence (10-14 days) before shaking at 150 RPM for 30 minutes to dislodge microglia.

FIG. 17. Primary cortical neurons can be treated with metabolites.

FIG. 18. Sholl analysis: a measurement of dendritic complexity.

FIG. 19. Treatment of neurons with metabolites increases dendritic complexity.

FIG. 20. Metabolite treatment alters excitatory vs inhibitory balance as visualized by staining for Bill-tubulin, VGlut1, gephyrin. The right most panel shows merged images.

FIG. 21. Metabolite treatment alters excitatory vs inhibitory balance with respect to gaphyrin and VGlut1

FIG. 22. Treatment of neurons with metabolites alters multiple mRNA species.

FIG. 23. Metabolite treatment induces microglial proliferation. Representative data of purified primary microglial cultures prepared as described in slide 2. Lefthand figure shows 3 DIV cultures stained for CD11b. Bottom figure shows FACS analysis of primary microglia stained for CX₃CR1 and CD11b directly after being shook off from confluent mixed glial cultures. Righthand figure shows after treatment with metabolites for 24 hours.

FIG. 24. Metabolite treatment induces microglial proliferation.

FIG. 25. Metabolite treatment increases presynaptic protein expression in cultured neurons.

FIG. 26. Representation of the outline of metabolite time course experiment utilizing BV-2 immortalized mouse microglia. BV-2 cells are plated with either media alone, or with a cocktail of the 4 metabolites identified in slide 1 at a final concentration of 32 ug/mL of each compound at the indicated time points. Cells are then harvested and stained using conventional methods for either cell surface markers or the proliferation marker Ki67.

FIG. 27. Metabolite treatment increases proliferative capacity and alters the cell surface profile of BV-2 cells. Results of metabolite time course in BV-2 cells. Top left figures are representative plots from BV-2 cells left untreated or treated for 5 days with the metabolite cocktail (+mets) before Ki67 staining and demonstrate increase proliferation after metabolite treatment. Top right figures are histograms of the MFI of the macrophage cell surface proteins CSF1R and F4/80 (previously demonstrated to be altered in microglia from germ-free mice when compared to SPF control conditions) demonstrating increased cell surface intensity of both markers after 5 days of metabolite treatment. The bottom row of figures shows quantification of above effects. Each bar is the average of 4 separate wells+/−SEM. P-vales calculated using two-way ANOVA.

FIG. 28. Inhibition of AhR reduces metabolite induced changes in surface markers in BV-2 cells but does not change proliferation. Results of AhR blockade on metabolite-induced proliferation and cell surface profile changes in BV-2 microglia. BV-2 cells were treated with vehicle (DMSO), metabolite cocktail (32 ug/mL of each metabolite), or metabolite cocktail with the AhR inhibitor CH223191 (5 uM final concentration in DMSO) for 5 days before performing the same analysis as in slide 5 (CSF1R and F4/80 or Ki67 staining). While the increase in F4/80 and CSF1R intensity is dependent on intact AhR signaling (top row), AhR signaling is dispensable for metabolite-induced proliferation (bottom row). Each bar is the average of 4 separate wells+/−SEM. P-vales calculated using two-way ANOVA.

FIG. 29. Indoxyl sulfate does not alter CSF1R expression or induce proliferation in BV-2 cells. This figure shows the results of indoxyl sulfate treatment on BV-2 microglia. In order to test the ability of the AhR ligand indoxyl sulfate in BV-2 microglia, cells were treated for 5 days with either a cocktail of metabolites or with indoxyl sulfate (32 ug/mL of all compounds tested). In contrast to a complete cocktail, indoxyl sulfate alone does not increase CSF1R or F4/80 protein staining intensity (top row) nor does it increase the proliferative capacity of BV-2 cells in culture (bottom row). Each bar is the average of 4 separate wells+/−SEM. P-vales calculated using two-way ANOVA.

FIG. 30. Metabolite treatment alters the cell surface profile of primary mouse microglia. Primary cultures of microglia were prepared as described in slide 2 and cultured with media alone (untreated) or with 32 ug/mL of metabolite cocktail for the indicated amount of time before staining with for CSF1R and F4/80. In contrast to BV-2 microglia, primary microglia demonstrate a reduction in the levels of CSF1R staining. Each bar is the average of 4 separate wells+/−SEM. P-vales calculated using two-way ANOVA.

FIG. 31. Metabolite treatment alters the phenotype of N2A cells. N2A cells were plated on poly-L-lysine coated coverslips in MEM with 0.1% FCS and P/S/Q and then further treated with either media alone or with a cocktail of 4 metabolites at a concentration of 32 ng/uL for 4 days before fixation and staining for the developmentally regulated neuronal specific tubulin isoform beta-III-tubulin (green) or the nuclear stain DAPI (blue). A representative image is shown in the left-hand micrographs. Cells were the imaged by conventional confocal microscopy, z-stacks generated, and the MFI of beta-III-tubulin in individual cells was measured (right-hand graph). A total of 16 20× fields was included for each condition (4 random 20× fields×4 individual wells per condition).

FIG. 32. Outline of phagocytosis assay in BV-2 cells. Briefly, apoptotic N2A cells (“bait”) are generated by UV irradiation and loaded with the pH sensitive dye CypHer5 before co-culture with Hoechst stained BV-2 cells. After washing, BV-2 cells are the collected and analyzed by flow cytometry. CD11b⁺ Hoechst⁺ BV-2 cells show an increase in CypHer5 intensity only upon engulfing apoptotic N2A cells while BV-2 cells that did not engulf bait cells remain CypHer5 low.

FIG. 33. Metabolite treatment increases BV-2 engulfment in vitro and is not dependent on AhR signaling. This figures shows the results of BV-2 phagocytosis assay. Control BV-2 alone do not demonstrate CypHer5 intensity, and intensity increases after co-culture with apoptotic N2A “bait” cells. Pretreatment with a cocktail of metabolites for 5 days prior to performing the phagocytosis assay significantly increases the percentage of CypHer5^{hi} microglia. This significance is abolished by co-incubation with the AhR inhibitor CH223191 (5 uM final concentration in DMSO). Each bar is the average of 4 separate wells +/−SEM. P-vales calculated using two-way ANOVA.

FIG. 34. Experimental outline for the acute depletion of microbiota in adult animals. 6 week-old male C57Bl6 mice were treated with drinking water alone (control SPF group), antibiotics (neomycin, vancomycin, metronidazole, ampicillin, gentamycin) in drinking water, or antibiotics plus a cocktail of the metabolites at a concentration of 32 ug/mL for a total of 2 weeks. The mice were then subjected to cue-dependent tone-shock fear conditioning. The FC assay consists of a single day conditioning regimen (3 tone/shock pairings, 0.5 mA shock) followed 3 recall blocks (5 trials per day×3 days total).

FIG. 35. Metabolite treatment rescues fear conditioning adult antibiotic-treated mice. This figures shows the results of metabolite exposure on acute microbiota depletion-mediated fear conditioning deficits. Groups of 6-8 mice were treated as outlined in slide 12 and then subjected with cue-dependent fear conditioning. The results of the last day of extinction training are shown in the left-hand figure. Differential freezing (percent of trial time freezing during session 1, trial 1-session 3, trial 15) of each group of mice is shown in the right-hand figure.

FIG. 36. Metabolite treatment alters the cell surface profile of microglia in vivo. After the last day of fear extinction, mice were sacrificed, and single cell suspensions were generated from the entire brain using mechanical dissociation followed by removal of myelin debris by density centrifugation. Cells were then stained and subjected to flow cytometry. Microglia (CD45^{int}, CD11b⁺, CX₃CR1^{hi}) demonstrate significantly higher levels of both CSF1R and MHCII surface staining intensity after treatment with a combination of antibiotics and metabolites when compared to untreated mice. Each group consists of 4 mice. Error is shown as +/−SEM, p-values calculated with one-way ANOVA.

FIG. 37. Metabolite treatment in the postnatal period rescues fear conditioning in germ-free mice. This figure shows the experimental outline and results of metabolite administration to germ-free mice starting a birth. The left-hand figures depict the experimental outline in which germ free mice are given a daily i.p injection of the metabolite cocktail (1.6 ug/g of body weight) from P1 through weaning before being moved into an SPF environment and recolonized with SPF microbiota. At P60, mice undergo a modified cue-dependent fear conditioning assay as depicted in the lower left figure. The right-hand figure demonstrates the results of this experiment in which the normal fear extinction defect seen in GF animals is rescued by the administration of metabolites (n=3 mice for SPF group and 7 for metabolites group, error+/−SEM).

FIGS. 38-40. Experimental outline (FIG. 38) and results (FIGS. 39 and 40) of transcript levels of multiple genes in either total medial prefrontal cortex (mPFC) tissue or sort-purified microglia after treatment of germ-free mice from P1-P28 with i.p. metabolites (1.6 ug/g of body weight every 3 days). After weaning mice were recolonized with SPF microbiota and tissues were collected at P56. Results are shown for a number of transcripts that were determined to be differentially regulated in neurons or microglia from antibiotic-treated mice by single nuclei RNA-seq in a prior study. Results are from 2-4 mice per group (SPF, GF treated with PBS, GF treated with metabolite cocktail) and are normalized to the house keeping gene Hprt. Error bars+/−SEM, p-values determined using student's t-test.

### DETAILED DESCRIPTION OF THE DISCLOSURE

Throughout this application, the use of the singular form encompasses the plural form and vice versa. For example, “a”, or “an” also includes a plurality of the referenced items, unless otherwise indicated.

Where a range of values is provided in this disclosure, it should be understood that each intervening value to the tenth decimal place of the lowest value, and all intervening ranges, between the upper and lower limit of that range is also included, unless clearly indicated otherwise. The upper and lower limits from within the broad range may independently be included in the smaller ranges encompassed within the disclosure.

The term “therapeutically effective amount” as used herein refers to an amount of an agent sufficient to achieve, in a single or multiple doses, the intended purpose of treatment. Treatment does not have to lead to complete cure, although it may. Treatment can mean alleviation of one or more of the symptoms or markers of the indication. The exact amount desired or required will vary depending on the particular compound or composition used, its mode of administration, patient specifics and the like. Appropriate effective amount can be determined by one of ordinary skill in the art informed by the instant disclosure using only routine experimentation. Within the meaning of the disclosure, “treatment” also includes prophylaxis and treatment of relapse, as well as the alleviation of acute or chronic signs, symptoms and/or malfunctions associated with the indication. Treatment can be orientated symptomatically, for example, to suppress symptoms. It can be effected over a short period, over a medium term, or can be a long-term treatment, such as, for example within the context of a maintenance therapy. Administrations may be intermittent, periodic, or continuous.

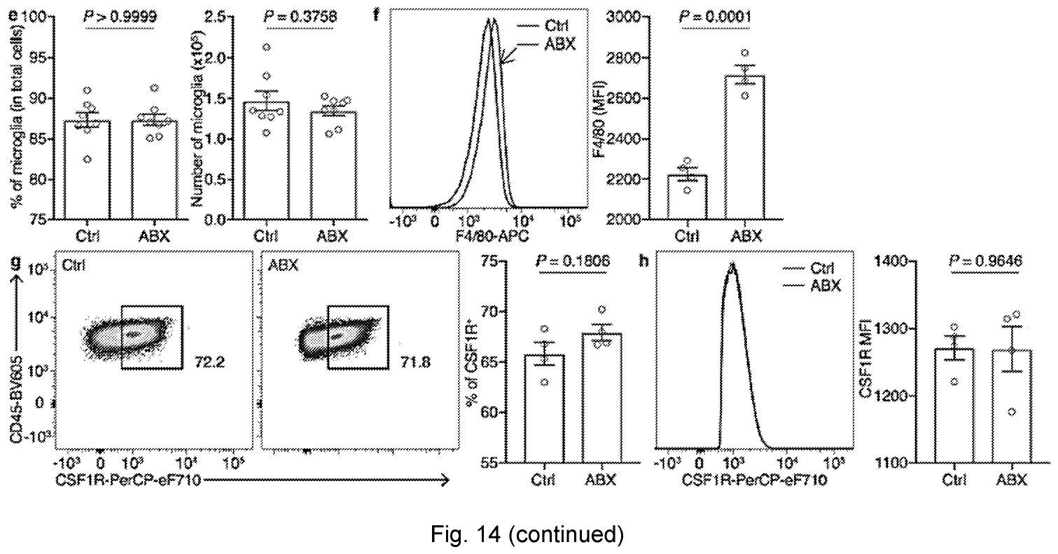

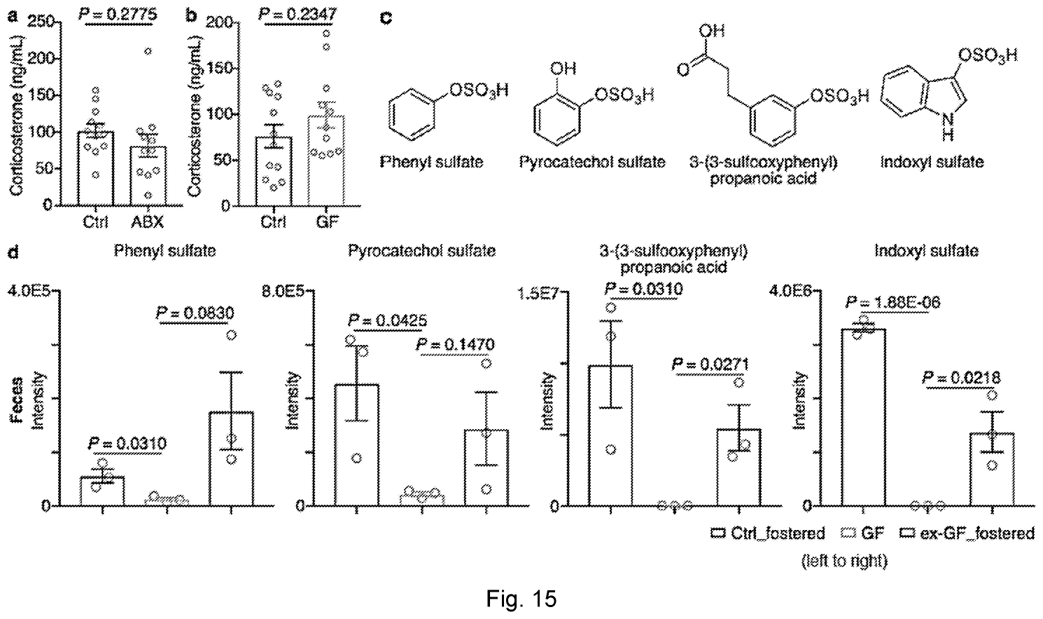

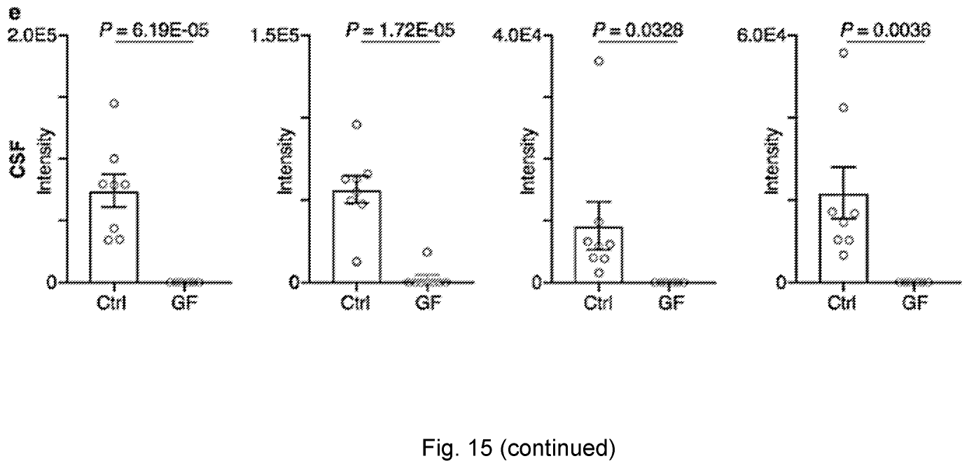

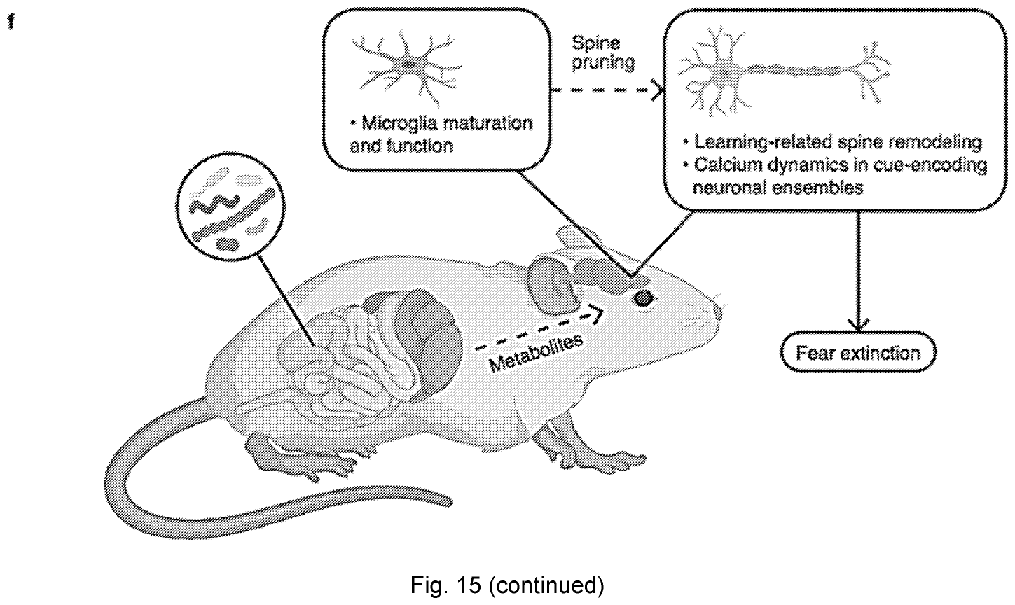

This disclosure describes that alterations in exposure to the microbiota in neonatal and adult mice can have profound and long-lasting effects on neuronal function and learning-related plasticity that subsequently regulate fear extinction behavior (FIG. 15f). From bulk RNA-seq and snRNA-seq data, the deficits in extinction learning correlate with malfunctions of the mPFC, notably in excitatory neurons. Transcranial two-photon live imaging confirmed the changes in neurons in the ABX mice—reduced extinction learning-associated spine formation and altered learning-related neuronal activity. Given that the vagus nerve does not contribute to the extinction learning deficits in ABX mice in this setting, the microbiota may affect the central nervous system through circulating microbiota-derived metabolites, directly influencing excitatory neurons in the mPFC, leading to deficits in extinction learning. In addition, microbiota-derived metabolites may also influence other cell subsets in the mPFC, such as microglia, and indirectly affect the excitatory neurons and behavior. In accordance with this, we found that the microglia in GF and ABX mice exhibit an immature state reminiscent of developing juvenile microglia, which may contribute to elevated spine pruning and reduced extinction learning-associated spine formation. Our findings provide a basis for the profound deficits in fear extinction learning in ABX and GF mice, and indicate alterations in microbiota-derived metabolites contribute to altered neuronal activity and behavior.

Extinction of classical fear conditioning refers to a reduction in conditional responding after the repeated presentation of a conditioned stimulus in the absence of the unconditioned stimulus with which it was previously paired. In experimental animals, extinction of fear conditioning is typically tested by using tone as a conditioned stimulus and footshock as an unconditioned stimulus.

This disclosure provides compositions and methods for treatment of neurological disorders and behavioral disorders. The compositions of the disclosure may be considered as dietary supplement compositions, probiotic compositions or pharmaceutical compositions, each of which may be referenced herein as “pharmaceutical composition”.

In an aspect, the present disclosure provides compositions comprising, consisting essentially of, or consisting of one or more of phenyl sulfate, pyrocatechol sulfate, 3-(3-sulfooxyphenyl)propanoic acid, and indoxyl sulfate. Structures of these compounds are shown in FIG. 15c. A reference to sulfate in this disclosure also includes the protonated form/free acid. A reference to 3-(3-sulfooxyphenyl)propanoic acid also includes partially or completely deprotonated form.

The structure of the compounds is shown below:

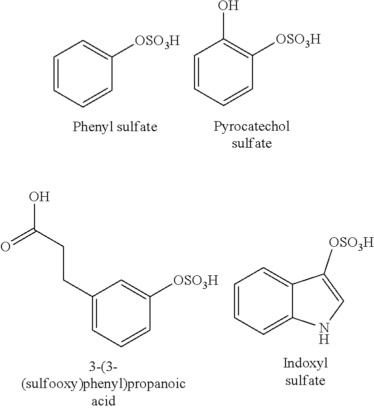

In an aspect, the present disclosure provides probiotic compositions comprising, consisting essentially of, or consisting of bacteria or combinations of bacteria that produce one or more of the following metabolites phenyl sulfate, pyrocatechol sulfate, 3-(3-sulfooxyphenyl)propanoic acid, and indoxyl sulfate.

In an embodiment, the present disclosure provides compositions comprising one or more of i) phenyl sulfate, pyrocatechol sulfate, 3-(3-sulfooxyphenyl)propanoic acid, and indoxyl sulfate, and ii) one or more bacteria that produce phenyl sulfate, pyrocatechol sulfate, 3-(3-sulfooxyphenyl)propanoic acid, and/or indoxyl sulfate.

In an embodiment, the disclosure provides a composition comprising, consisting essentially of, or consisting of phenyl sulfate, pyrocatechol sulfate, 3-(3-sulfooxyphenyl)propanoic acid, and indoxyl sulfate.

In an embodiment, the disclosure provides a composition comprising, consisting essentially of, or consisting of phenyl sulfate and pyrocatechol sulfate.

In an embodiment, the disclosure provides a composition comprising, consisting essentially of, or consisting of phenyl sulfate and 3-(3-sulfooxyphenyl)propanoic acid.

In an embodiment, the disclosure provides a composition comprising, consisting essentially of, or consisting of phenyl sulfate and indoxyl sulfate.

In an embodiment, the disclosure provides a composition comprising, consisting essentially of, or consisting of pyrocatechol sulfate and 3-(3-sulfooxyphenyl)propanoic acid.

In an embodiment, the disclosure provides a composition comprising, consisting essentially of, or consisting of pyrocatechol sulfate and indoxyl sulfate.

In an embodiment, the disclosure provides a composition comprising, consisting essentially of, or consisting of 3-(3-sulfooxyphenyl)propanoic acid and indoxyl sulfate.

In an embodiment, the disclosure provides a composition comprising, consisting essentially of, or consisting of a compound or compounds that can be metabolized in vivo (such as after administration to a subject, e.g., a human) to phenyl sulfate, pyrocatechol sulfate, 3-(3-sulfooxyphenyl)propanoic acid or indoxyl sulfate.

The phenyl sulfate, pyrocatechol sulfate, 3-(3-sulfooxyphenyl)propanoic acid, and indoxyl sulfate, each or together, may be present in the present compositions in amounts ranging from 1 ng/kg to about 100 mg/kg including all values and ranges therebetween. In an embodiment, the compounds/metabolites, each or together, may be present in amounts ranging from 0.1 μg/kg to 50 mg/kg. In an embodiment, the compounds/metabolites, each or together, may be present in amounts ranging from 1 μg/kg to about 10 mg/kg and all values and ranges therebetween. In an embodiment, the amounts of the metabolites in the compositions may be adjusted to restore the concentrations of the metabolites to their normal physiologic levels. Normal physiological levels can be obtained from a population of normal individuals with respect to neurological disorders (i.e. from those individuals who are not afflicted with any neurological disorders) and these values can be normalized for age, gender, and other population features.

In an embodiment, the compositions may be probiotic compositions that increase the amounts of one or more of the following in the animal: phenyl sulfate, pyrocatechol sulfate, 3-(3-sulfooxyphenyl)propanoic acid, and indoxyl sulfate, and may include bacteria that produce one or more of these compounds. In an embodiment, the compositions may be probiotic compositions that increase the amounts of at least two, at least three, or all four of the following in the animal: phenyl sulfate, pyrocatechol sulfate, 3-(3-sulfooxyphenyl)propanoic acid, and indoxyl sulfate, and may include bacteria that individually or collectively produce one or more of these compounds. Examples of bacteria that increase the amounts of phenyl sulfate, pyrocatechol sulfate, 3-(3-sulfooxyphenyl)propanoic acid, and/or indoxyl sulfate include Clostridium or Bacteroides species.

In an embodiment, the compositions may be probiotic compositions, comprising, consisting essentially of, or consisting of bacteria from the Clostridium or Bacteroides species. The bacteria are preferably not those which are associated with diseased conditions. In an embodiment, the compositions comprise, consist essentially of, or consist of non-pathogenic bacteria from the Clostridium or Bacteroides species. These may be gut-commensal bacteria. For example in embodiments, the compositions are free of C. difficile. In embodiments, the Clostridium and/or Bacteroides species may be the only bacteria in the probiotic composition. The bacteria may be one or more of Bacteroides uniformis ATCC 8492 MAF100 uniformis ATCC 8492, Bacteroides 7 sp 4 1 36, Bacteroides 36 oleiciplenus YIT 12058, Bacteroides theraiotaomicron VPI-5482 MAF100 thetaiotaomicron VPI-5482, Bacteroides sp. 1 1 6 MAF100 sp. 116, Bacteroides 14 sp. 1 1 14, Bacteroides 31 ovatus SD CMC 3f, Bacteroides 15 sp. 3 1 23, Bacteroides ovatus ATCC 8483 MAF100 ovatus ATCC 8483, Bacteroides 27 ovatus 3 8 47FAA, Bacteroides 56 sp. 2 2 4, Bacteroides 8 sp. 1 1 30, Bacteroides sp. D22, Bacteroides 32 xylanisolvens SD CC 2a, Bacteroides 29 xylanisolvens CL03T12C04, Bacteroides 47 xylanisolvens SD CC 1b, Bacteroides sp. 2 1 22 MAF100 sp. 2 1 22, Bacteroides 4 sp. D1, Clostridium sporogenes DSM795 MAF100 Clostridium sporogenes strain DSM795 complete genome, Clostridium sporogenes ATCC15579 MAF100 sporogenes ATCC 15579, Clostridium asparagiforme DSM 15981 MAF100 Clostridium asparagiforme DSM 15981 genomic scaffold Scfld7 whole genome shotgun sequence, and Clostridium saccharolyticum WM1 MAF100 Clostridium saccharolyticum WM1 complete sequence. These may be the only bacteria in the probiotic composition or other bacteria may be added.

The amount of bacteria per dose may be (together or individually for each type of bacteria) 100 million to 1 billion and all values and ranges therebetween. In an embodiment, a dose may have more than 1 billion bacteria. A dose may be a tablet, capsule, or a specified amount of the formulation in any form. In various embodiments, the bacteria per dose may be 100, 200, 300, 400, 500, 600, 700, 800, 900 million or 1 billion, 2 billion, 3, billion etc.

In an embodiment, the disclosure provides compositions, such as probiotic compositions comprising or consisting essentially of i) one or more of phenyl sulfate, pyrocatechol sulfate, 3-(3-sulfooxyphenyl)propanoic acid, and indoxyl sulfate, and ii) bacteria from the Clostridium or Bacteroides species, which are not pathogenic bacteria.

In an embodiment, the composition comprises i) one or more of phenyl sulfate, pyrocatechol sulfate, 3-(3-sulfooxyphenyl)propanoic acid, and indoxyl sulfate, and ii) one or more of Bacteroides uniformis ATCC 8492 MAF100 uniformis ATCC 8492, Bacteroides 7 sp 4 1 36, Bacteroides 36 oleiciplenus YIT 12058, Bacteroides theraiotaomicron VPI-5482 MAF100 thetaiotaomicron VPI-5482, Bacteroides sp. 1 1 6 MAF100 sp. 116, Bacteroides 14 sp. 1 1 14, Bacteroides 31 ovatus SD CMC 3f, Bacteroides 15 sp. 3 1 23, Bacteroides ovatus ATCC 8483 MAF100 ovatus ATCC 8483, Bacteroides 27 ovatus 3 8 47FAA, Bacteroides 56 sp. 2 2 4, Bacteroides 8 sp. 1 1 30, Bacteroides sp. D22, Bacteroides 32 xylanisolvens SD CC 2a, Bacteroides 29 xylanisolvens CL03T12C04, Bacteroides 47 xylanisolvens SD CC 1b, Bacteroides sp. 2 1 22 MAF100 sp. 2 1 22, Bacteroides 4 sp. D1, Clostridium sporogenes DSM795 MAF100 Clostridium sporogenes strain DSM795 complete genome, Clostridium sporogenes ATCC15579 MAF100 sprorogenes ATCC 15579, Clostridium difficile NAP08-16S rRNA Clostridium difficile NAP08 genomic scaffold SCAFFOLDS whole genome shotgun sequence, Clostridium difficile NAP07-16S rRNA Clostridium difficile NAP07 genomic scaffold SCAFFOLD13, Clostridium asparagiforme DSM 15981 MAF100 Clostridium asparagiforme DSM 15981 genomic scaffold Scfld7 whole genome shotgun sequence, and Clostridium saccharolyticum WM1 MAF100 Clostridium saccharolyticum WM1 complete sequence.

The bacterial compositions of the present disclosure may be present as pharmaceutically acceptable compositions comprising therapeutically-effective amount(s) of one or more of the bacterial species that increase the amounts of one or more, two or more, three or more, or all four of phenyl sulfate, pyrocatechol sulfate, 3-(3-sulfooxyphenyl)propanoic acid, and indoxyl sulfate. The bacteria may be formulated with pharmaceutically acceptable excipients and other therapeutically effective medications known in the art allowing for but not limited to combination therapies to improve overall efficacy of each individual therapeutic or to limit the concentration of either therapeutic to avoid side effects and maintain efficacy. The bacteria may be isolated from their natural environment and may be present with or without pharmaceutically acceptable excipients and carriers in a lyophilized, freeze-dried, solid or powdered forms. The bacteria and excipient(s) may be formulated into compositions and dosage forms according to methods known in the art. The bacterial pharmaceutical compositions may be specially formulated for administration in solid, liquid or aerosolized form, including those adapted for the following: oral administration, for example, tablets, capsules, powders, granules, and aqueous or non-aqueous solutions or suspensions, drenches, or syrups, frozen or freeze-dried forms; or intrarectally, for example, as a pessary, cream or foam, or aerosols for intranasal administration. In an embodiment, the only bacteria present in the probiotic compositions are those which produce one or more of phenyl sulfate, pyrocatechol sulfate, 3-(3-sulfooxyphenyl)propanoic acid, and/or indoxyl sulfate.

The metabolites may be present as pharmaceutically acceptable compositions comprising therapeutically-effective amount(s) of one or more of phenyl sulfate, pyrocatechol sulfate, 3-(3-sulfooxyphenyl)propanoic acid, and indoxyl sulfate or prodrugs that increase the amounts of one or more of the following in the animal: phenyl sulfate, pyrocatechol sulfate, 3-(3-sulfooxyphenyl)propanoic acid, and indoxyl sulfate, formulated together with one or more pharmaceutically acceptable excipients or other therapeutically effective medications known in the art allowing for but not limited to combination therapies to improve overall efficacy of each individual therapeutic or to limit the concentration of either therapeutic to avoid side effects and maintain efficacy. The active ingredient and excipient(s) may be formulated into compositions and dosage forms according to methods known in the art. The pharmaceutical compositions of the present disclosure may be specially formulated for administration in solid or liquid form, including those adapted for the following: (1) oral administration, for example, tablets, capsules, powders, granules, pastes for application to the tongue, aqueous or non-aqueous solutions or suspensions, drenches, or syrups; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

An effective amount of the pharmaceutical composition of the present disclosure is sufficient to promote cognitive, neurological, or psychological health, or to treat prevent a disease or condition comprising a cognitive disorder, a neurological disorder, an anxiety disorder, including an anxiety disorder characterized by extinction learning deficits, or other psychological disorder. The dosage of active ingredient(s) may vary, depending on the reason for use and the individual subject. The dosage may be adjusted based on the subject's weight, the age and health of the subject, and tolerance for the compound or composition.

The phrase “pharmaceutically acceptable” is used herein to refer to those compounds, materials, compositions, and/or dosage forms which are, within the scope of sound medical judgment, suitable for use in contact with the tissues of the subject with toxicity, irritation, allergic response, or other problems or complications, commensurate with a reasonable benefit/risk ratio.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

Excipients are added to the composition for a variety of purposes. Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the subject's stomach may be increased by the addition of a disintegrant to the composition. Disintegrants include alginic acid, carboxymethylcellulose calcium, carboxymethylcellulose sodium (e.g. Ac Di Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

In liquid pharmaceutical compositions of the present disclosure, the therapeutically-effective amount of one or more of phenyl sulfate, pyrocatechol sulfate, 3-(3-sulfooxyphenyl)propanoic acid, and indoxyl sulfate or probiotic composition and any other solid excipients are dissolved or suspended in a liquid carrier such as water, water-for-injection, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present disclosure include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present disclosure may also contain a viscosity enhancing agent to improve the mouth feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste. Flavoring agents and flavor enhancers may make the dosage form more palatable to the patient. Common flavoring agents and flavor enhancers for pharmaceutical products that may be included in the composition of the present disclosure include maltol, vanillin, ethyl vanillin, menthol, citric acid, fumaric acid, ethyl maltol and tartaric acid.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present disclosure, a liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

Solid and liquid compositions may also be dyed using any pharmaceutically acceptable colorant to improve their appearance and/or facilitate patient identification of the product and unit dosage level.

The dosage form of the present disclosure may be a capsule containing the composition, for example, a powdered or granulated solid composition of the disclosure, within either a hard or soft shell. The shell may be made from gelatin and optionally contain a plasticizer such as glycerin and sorbitol, and an opacifying agent or colorant.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling may include any of the aforementioned blends and granulates that were described with reference to tableting, however, they are not subjected to a final tableting step.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Micelles

The pharmaceutical industry introduced microemulsification technology to improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

In one aspect of disclosure, the formulations contain micelles comprising one or more compounds and/or bacteria of the present disclosure and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present disclosure and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Particularly preferred amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

Polymers

Hydrophilic polymers suitable for use in the present disclosure are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Preferred polymers are those having a molecular weight of from about 100 or 120 daltons up to about 5,000 or 10,000 daltons, and more preferably from about 300 daltons to about 5,000 daltons. In a particularly preferred embodiment, the polymer is polyethyleneglycol having a molecular weight of from about 100 to about 5,000 daltons, and more preferably having a molecular weight of from about 300 to about 5,000 daltons. In a particularly preferred embodiment, the polymer is polyethyleneglycol of 750 daltons (PEG(750)). Polymers may also be defined by the number of monomers therein; a preferred embodiment of the present disclosure utilizes polymers of at least about three monomers, such PEG polymers consisting of three monomers (approximately 150 daltons).

Other hydrophilic polymers which may be suitable for use in the present disclosure include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In certain embodiments, the compositions comprise polymers and one or more compounds and/or bacteria of the present disclosure.

Cyclodextrins

Cyclodextrins are cyclic oligosaccharides, consisting of 6, 7 or 8 glucose units, designated by the Greek letter alpha, beta, or gamma, respectively. Cyclodextrins with fewer than six glucose units are not known to exist. The glucose units are linked by alpha-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17-beta-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259, hereby incorporated herein by reference) and Gramera, et al. (U.S. Pat. No. 3,459,731, hereby incorporated herein by reference) described electroneutral cyclodextrins. Other derivatives include cyclodextrins with cationic properties [Parmeter (II), U.S. Pat. No. 3,453,257, hereby incorporated herein by reference], insoluble crosslinked cyclodextrins (Solms, U.S. Pat. No. 3,420,788, hereby incorporated herein by reference), and cyclodextrins with anionic properties [Parmeter (III), U.S. Pat. No. 3,426,011, hereby incorporated herein by reference]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127, hereby incorporated herein by reference).

In certain embodiment, the composition comprises cyclodextrins and one or more compounds and/or bacteria of the present disclosure.

Liposomes

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present disclosure relates to formulations comprising liposomes encapsulating or having incorporated therein one or more compounds and/or bacteria of the present disclosure, where the liposome membrane is formulated to provide a liposome with increased carrying capacity. Alternatively or in addition, the compound of the present disclosure may be contained within, or adsorbed onto, the liposome bilayer of the liposome. The compound of the present disclosure may be aggregated with a lipid surfactant and carried within the liposome's internal space; in these cases, the liposome membrane is formulated to resist the disruptive effects of the active agent-surfactant aggregate.

According to one embodiment of the present disclosure, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present disclosure are in solubilized form. Aggregates of surfactant and active agent (such as emulsions or micelles containing the active agent of interest) may be entrapped within the interior space of liposomes according to the present disclosure. A surfactant acts to disperse and solubilize the active agent, and may be selected from any suitable aliphatic, cycloaliphatic or aromatic surfactant, including but not limited to biocompatible lysophosphatidylcholines (LPCs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMCs in the micromolar range; higher CMC surfactants may be utilized to prepare micelles entrapped within liposomes of the present disclosure, however, micelle surfactant monomers could affect liposome bilayer stability and would be a factor in designing a liposome of a desired stability.

Liposomes according to the present disclosure may be prepared by any of a variety of techniques that are known in the art. See, e.g., U.S. Pat. No. 4,235,871; Published PCT applications WO 96/14057, both of which are hereby incorporated herein by reference; New RRC, Liposomes: A practical approach, IRL Press, Oxford (1990), pages 33-104; Lasic DD, Liposomes from physics to applications, Elsevier Science Publishers BV, Amsterdam, 1993.

For example, liposomes of the present disclosure may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present disclosure, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323, hereby incorporated herein by reference

Release Modifiers

The release characteristics of a formulation of the present disclosure depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In all cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween™ and Pluronic™. Pore forming agents which add microstructure to the matrices (i.e., water soluble compounds such as inorganic salts and sugars) are added as particulates. The range should be between one and thirty percent (w/w polymer).

In certain embodiments, the compositions comprises release modifiers and one or more compounds and/or bacteria of the present disclosure.

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates.

In an aspect, this disclosure provides methods for promoting the neurological health, psychological health or brain health of an individual comprising contacting the individual with or administering to the individual in need of treatment a probiotic composition comprising, consisting essentially of, or consisting of one or more i) bacteria which produce one or more of phenyl sulfate, pyrocatechol sulfate, 3-(3-sulfooxyphenyl)propanoic acid, and indoxyl sulfate, or ii) one or more of phenyl sulfate, pyrocatechol sulfate, 3-(3-sulfooxyphenyl)propanoic acid, and indoxyl sulfate, or a compositing a prodrug that can be converted to one or more of phenyl sulfate, pyrocatechol sulfate, 3-(3-sulfooxyphenyl)propanoic acid, and indoxyl sulfate, or a combination of i) and ii). In an embodiment, the method comprises or consists essentially of administering to an individual in need of treatment a composition comprising, consisting essentially or consisting of one or more of phenyl sulfate, pyrocatechol sulfate, 3-(3-sulfooxyphenyl)propanoic acid, and indoxyl sulfate, and/or one or more non-pathogenic Clostridium or Bacteroides species.

The phenyl sulfate, pyrocatechol sulfate, 3-(3-sulfooxyphenyl)propanoic acid, and indoxyl sulfate may be administered together or separately in amounts ranging from 1 ng/kg to about 100 mg/kg each including all values and ranges therebetween. In an embodiment, the compounds/metabolites may be administered in amounts ranging from 0.1 μg/kg to 50 mg/kg. In an embodiment, the compounds/metabolites may be administered in amounts ranging from 1 μg/kg to about 10 mg/kg and all values and ranges therebetween.

The bacteria administered per dose may be (together or individually for each type of bacteria) 100 million to 1 billion and all values and ranges therebetween. In an embodiment, a dose may have more than 1 billion bacteria. A dose may be a tablet, capsule, or a specified amount of the formulation in any form. In various embodiments, the bacteria per dose may be 100, 200, 300, 400, 500, 600, 700, 800, 900 million or 1 billion, 2 billion, 3, billion etc

In an embodiment, the disclosure provides method of providing to an individual in need of treatment phenyl sulfate, pyrocatechol sulfate, 3-(3-sulfooxyphenyl)propanoic acid, and/or indoxyl sulfate, comprising administering to the individual a compound or compounds that can be metabolized in vivo (such as after administration to a subject, e.g., a human) to phenyl sulfate, pyrocatechol sulfate, 3-(3-sulfooxyphenyl)propanoic acid, and indoxyl sulfate.

The individual in need of treatment may be an individual afflicted with a neurological condition. The individual may be an individual treated with an antibiotic. In an embodiment, the present compositions may be administered together with an antibiotic therapeutic regimen. For example the present compositions may be administered just prior to, concomitant with (partial or complete), or after termination of the antibiotic treatment regimen. The individual may be a newborn born via a non-vaginal delivery, such as C-section, in which case, the individual may be contacted with the present compositions in the form of a wash. In an embodiment, the contacting or administration of the present compositions is carried out soon after birth. The individual may be afflicted with autism or neurodegenerative diseases.

In an embodiment, the present compositions may be administered as companion treatment with antibiotic treatment for an individual of any age. One or more doses of the present metabolite compositions may be administered prior to, concurrently, or subsequent to an antibiotic treatment regimen. The present compositions may be particularly useful as companion treatments for the stronger antibiotics that are known to affect gut microbiota.

In an embodiment, the present compositions may be administered to an individual whose gut microbiota has been altered due to disease, trauma, medication (including antibiotic or chemotherapy). One or more doses of the present metabolite compositions may be administered prior to, concurrently, or subsequent to a medication regimen or surgery, or disease. One or more doses of the present metabolite compositions may be administered at a suitable time after trauma or disease onset.

An individual may be administered the present compositions over a suitable period of time, which may be days, weeks, months or on a prolonged basis over years. The frequency of administrations may be determined by a clinician. Circulating levels may be measured to adjust dosage so that levels are at or near normal physiological levels.

In an embodiment, the disclosure provides a method for enhancing the proliferation, function or activity of brain cells, e.g., microglia and neurons, comprising administering to an individual in need of treatment a composition comprising, consisting essentially of, or consisting of one or more i) bacteria which produce one or more of phenyl sulfate, pyrocatechol sulfate, 3-(3-sulfooxyphenyl)propanoic acid, and indoxyl sulfate, or ii) one or more of phenyl sulfate, pyrocatechol sulfate, 3-(3-sulfooxyphenyl)propanoic acid, and indoxyl sulfate, or a compositing a prodrug that can be converted to one or more of phenyl sulfate, pyrocatechol sulfate, 3-(3-sulfooxyphenyl)propanoic acid, and indoxyl sulfate, or a combination of i) and ii). The bacteria may be one or more non-pathogenic Clostridium or Bacteroides species.

In an embodiment, this disclosure provides a method for facilitating extinction learning comprising administering to an individual in need of treatment a composition comprising, consisting essentially of, or consisting of one or more of i) bacteria which produce one or more of phenyl sulfate, pyrocatechol sulfate, 3-(3-sulfooxyphenyl)propanoic acid, and indoxyl sulfate, or ii) one or more of phenyl sulfate, pyrocatechol sulfate, 3-(3-sulfooxyphenyl)propanoic acid, and indoxyl sulfate, or a compositing a prodrug that can be converted to one or more of phenyl sulfate, pyrocatechol sulfate, 3-(3-sulfooxyphenyl)propanoic acid, and indoxyl sulfate, or a combination of i) and ii). An individual in need of facilitating extinction learning may be an individual with anxiety disorders, such as panic disorder and posttraumatic stress disorder (PTSD).

In an embodiment, the disclosure provides a method for treating or ameliorating the symptoms of extinction learning deficits, or other disorder including defects in learning/cognitive function, autism, neurodegenerative diseases including Parkinsons and Alzheimers comprising administering to an individual in need of treatment a composition comprising, consisting essentially of, or consisting of one or more i) bacteria which produce one or more of phenyl sulfate, pyrocatechol sulfate, 3-(3-sulfooxyphenyl)propanoic acid, and indoxyl sulfate, or ii) one or more of phenyl sulfate, pyrocatechol sulfate, 3-(3-sulfooxyphenyl)propanoic acid, and indoxyl sulfate, or a compositing a prodrug that can be converted to one or more of phenyl sulfate, pyrocatechol sulfate, 3-(3-sulfooxyphenyl)propanoic acid, and indoxyl sulfate, or a combination of i) and ii). The bacteria may be one or more non-pathogenic Clostridium or Bacteroides species.

The subject (alternatively referred to herein as an “individual”) may be any animal, including human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Preferred subjects include human subjects for whom it is desirable to promote their cognitive, neurological, or psychological health, or who are suffering from or at risk for a disease or condition comprising a cognitive disorder, a neurological disorder, a developmental disorder, an anxiety disorder, including an anxiety disorder characterized by extinction learning deficits, or other disorder including defects in learning/cognitive function, autism, neurodegenerative diseases including Parkinsons and Alzheimers, and pre-term births with altered microbiota exposure. The subject is generally diagnosed with the condition of the subject disclosure by skilled artisans, such as a medical practitioner.

The methods of the disclosure described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, infants, and newborns. Examples of human ethnic populations include Caucasians, Asians, Hispanics, Africans, African Americans, Native Americans, Semites, and Pacific Islanders. The methods of the disclosure may be more appropriate for some ethnic populations such as Caucasians, especially northern European populations, as well as Asian populations. The term subject also includes subjects of any genotype or phenotype as long as they are in need of the methods/treatments, as described herein. In addition, the subject can have the genotype or phenotype for any hair color, eye color, skin color or any combination thereof. The term subject includes a subject of any body height, body weight, or any organ or body part size or shape.

In embodiments, the disclosure provides the following illustrative methods.

Illustrative method 1) A method to promote an animal's psychological health by administering to said animal a composition comprising or consisting essentially of one or more of the following: phenyl sulfate, pyrocatechol sulfate, 3-(3-sulfooxyphenyl)propanoic acid, and indoxyl sulfate.

Illustrative method 2) A method to promote an animal's psychological health by administering to said animal a probiotic composition that increases the amounts of one or more of the following in the animal: phenyl sulfate, pyrocatechol sulfate, 3-(3-sulfooxyphenyl)propanoic acid, and indoxyl sulfate.

Illustrative method 3) The method of illustrative method 1 or 2, wherein the levels of said compounds are increased in serum, cerebrospinal fluid, or fecal matter excreted by said animal.

Illustrative method 4) A method to treat an anxiety disorder in an animal, by administering to said animal a composition comprising or consisting essentially of one or more of the following: phenyl sulfate, pyrocatechol sulfate, 3-(3-sulfooxyphenyl)propanoic acid, and indoxyl sulfate.

Illustrative method 5) The method of Illustrative method 4, wherein the anxiety disorder is associated with a deficit in extinction learning.

Illustrative method 6) A method to treat an anxiety disorder in an animal by administering to said animal a probiotic composition that increases the amounts of one or more of the following in the animal: phenyl sulfate, pyrocatechol sulfate, 3-(3-sulfooxyphenyl)propanoic acid, and indoxyl sulfate.

Illustrative method 7) The method of Illustrative method 6, wherein the levels of said compounds are increased in serum, cerebrospinal fluid, or fecal matter excreted by said animal.

The invention if further described in the examples provided below, which are intended to be illustrative and not restrictive in any way.

### Example 1

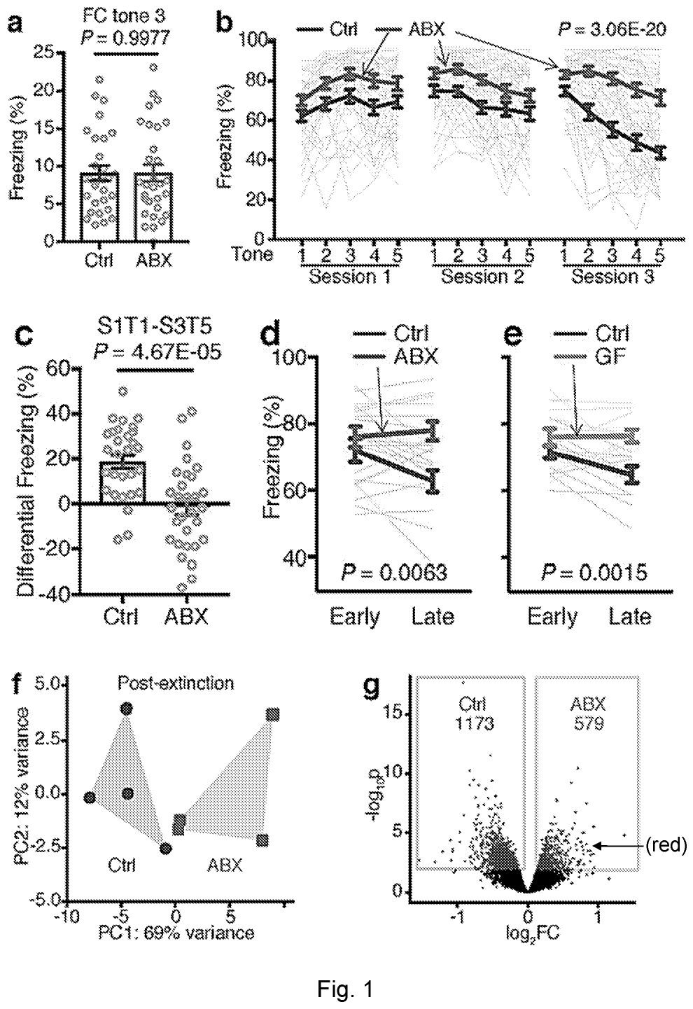

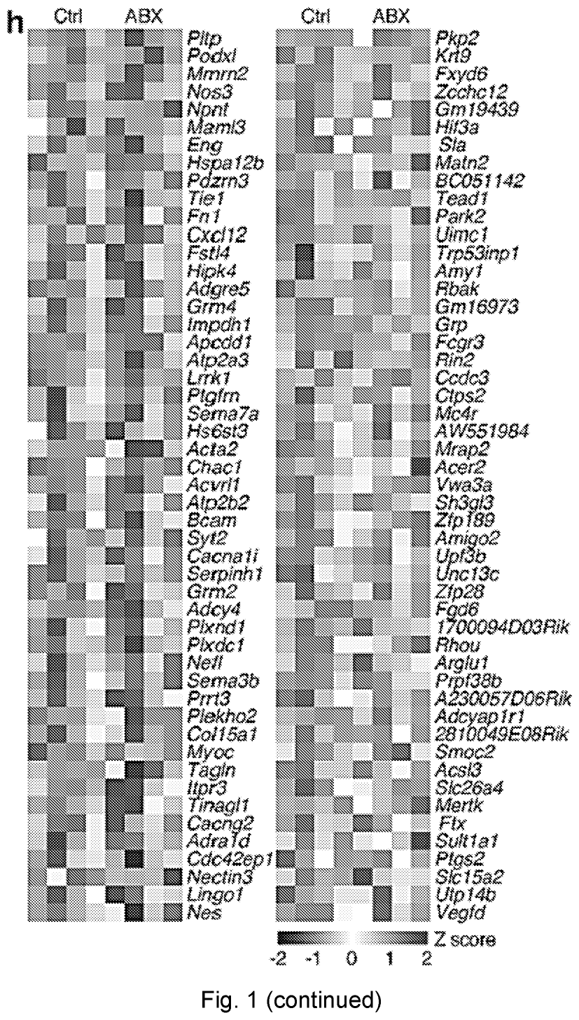

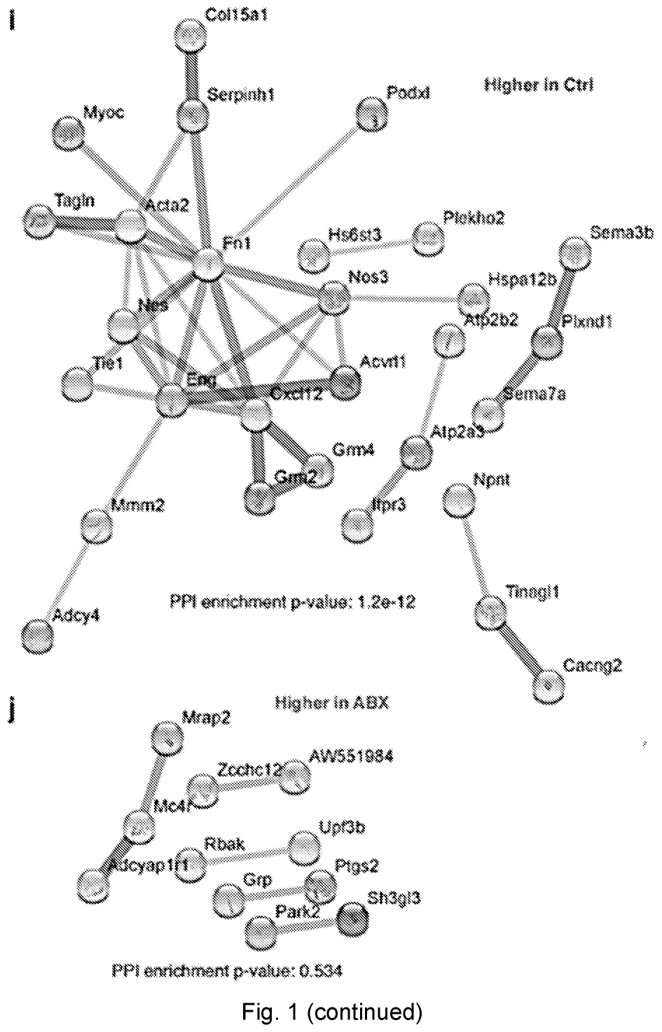

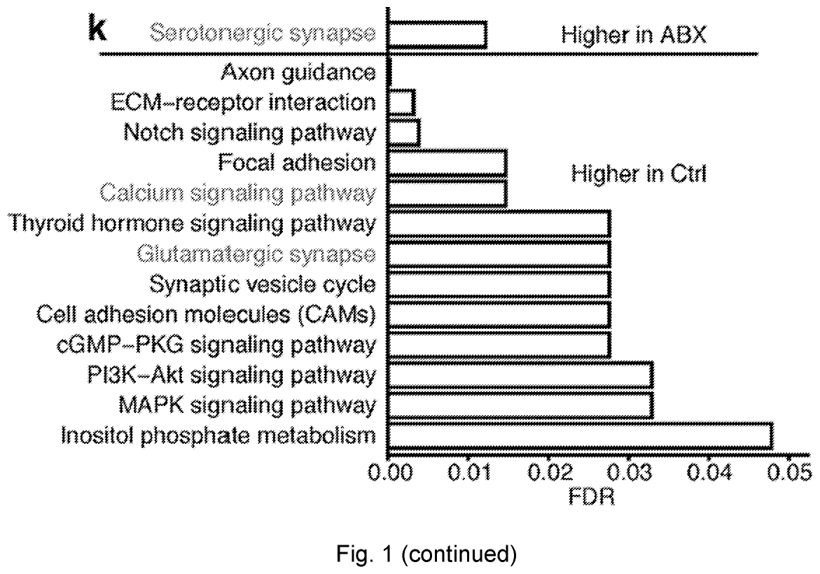

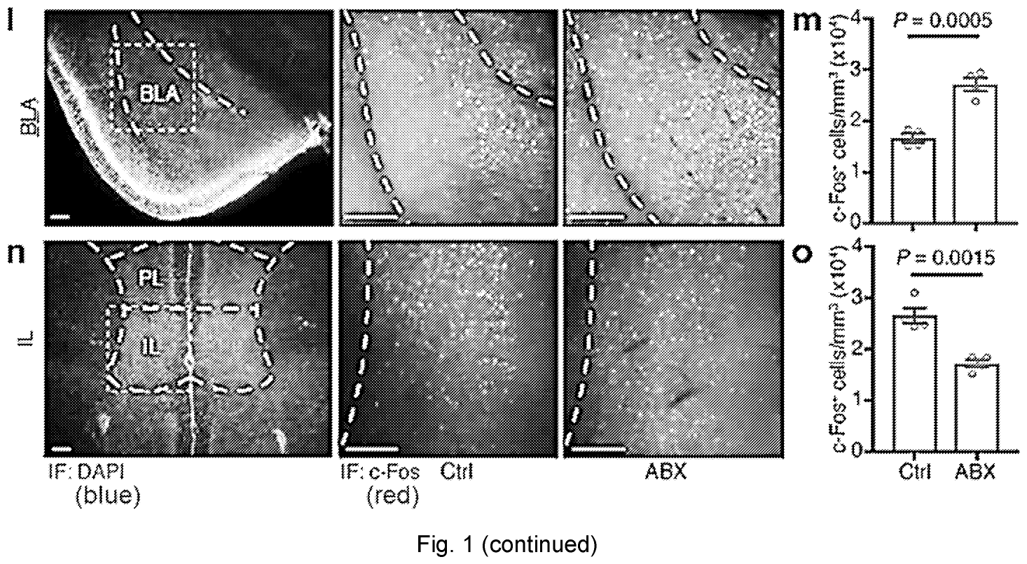

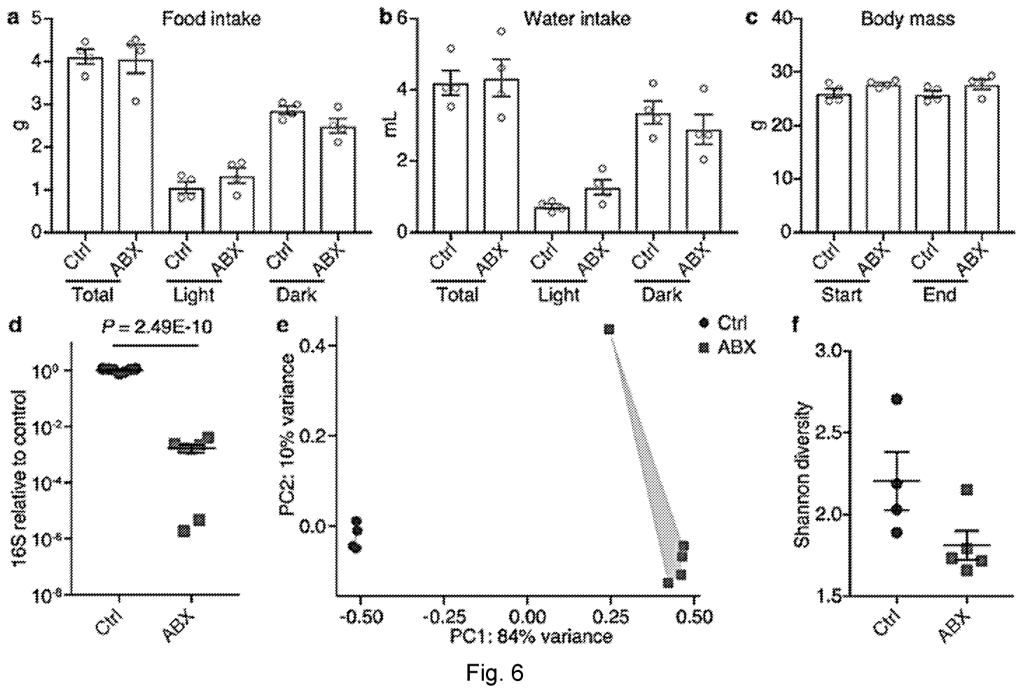

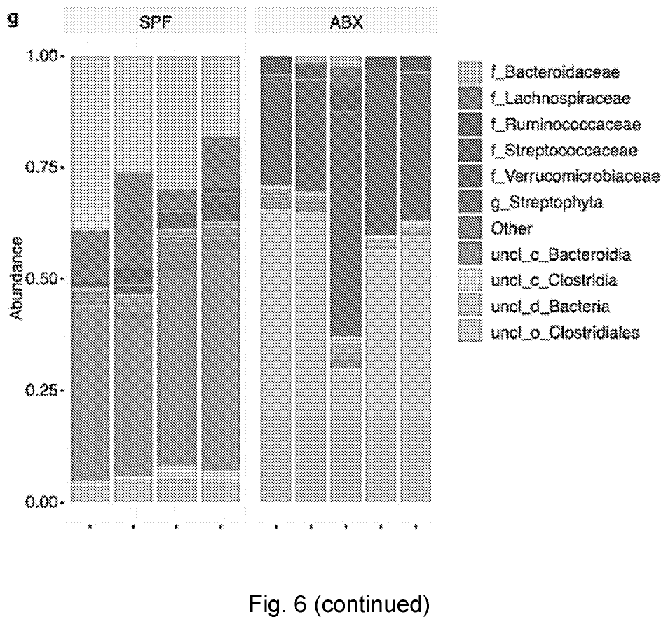

Extinction learning deficits following manipulation of the microbiota. To test whether the microbiota influence fear conditioning and extinction, first we tested antibiotic-treated adult mice (termed ABX mice) using a classical cued fear conditioning and extinction learning paradigm. ABX mice and control (Ctrl) mice showed comparable food/water intake and weight gain (FIG. 6a-c). The bacterial burden was 600-fold lower in ABX mice compared to Ctrl mice (FIG. 6d), and 16S rDNA sequencing revealed a shift in bacterial community structure due to the antibiotic treatment (FIG. 6e-g). Following fear conditioning, ABX mice displayed equivalent freezing behavior to Ctrl mice, indicating that the acquisition of fear conditioning was normal (FIG. 1a). Extinction learning reduced conditioned freezing in Ctrl mice. In contrast, extinction learning was significantly impaired in ABX mice (FIG. 1b, c). To further examine the influence of the microbiota on extinction learning, we performed a similar cued fear conditioning and extinction learning assay in adult germ-free (GF) mice. To maintain the microbe-free status of the GF mice, we used a modified single-session fear extinction protocol (Soliman et al., 2010, Science 327, 863-866). Again, both ABX and GF mice exhibited impaired extinction learning (FIG. 1d, e). These data demonstrate that signals derived from the microbiota are indispensable for optimal extinction of conditioned fear responses.

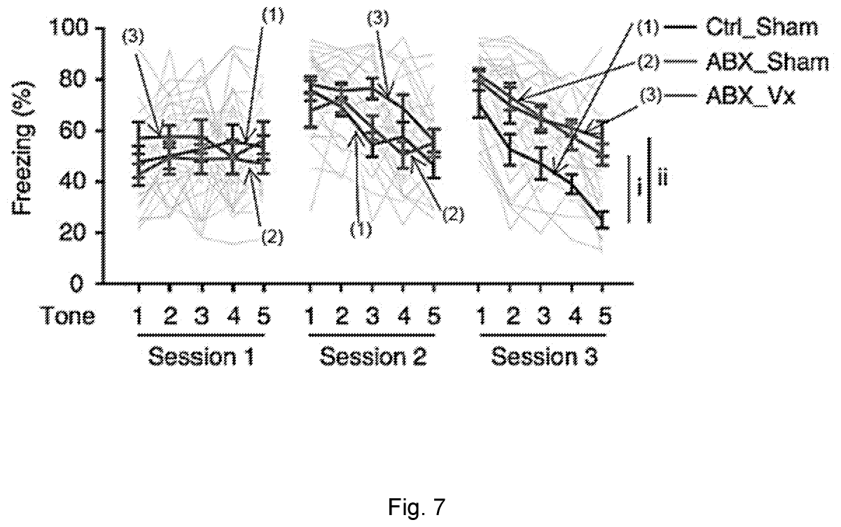

The vagus nerve is one mechanism through which neuronal communication between the intestine and the brain is established. We sought to test whether the vagus nerve is involved in extinction learning deficits following deliberate manipulation of the microbiota by employing surgical vagotomy in adult animals. Notably, vagotomized ABX mice exhibited similar deficits in extinction learning as Sham ABX mice, suggesting that the extinction learning deficits in ABX mice is vagus nerve-independent (FIG. 7).

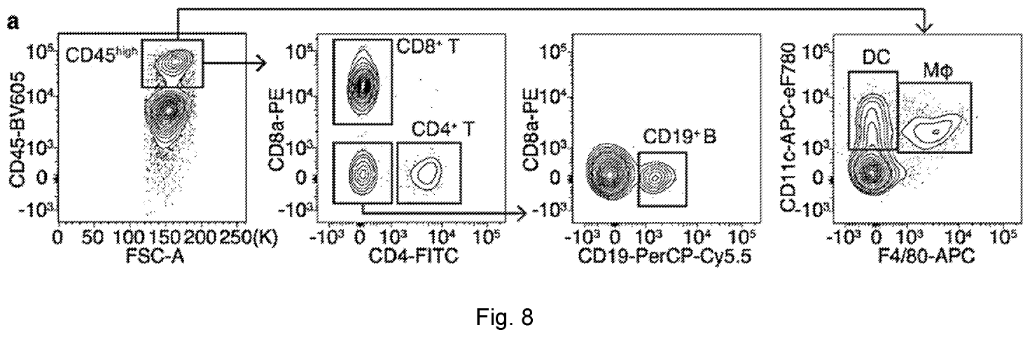

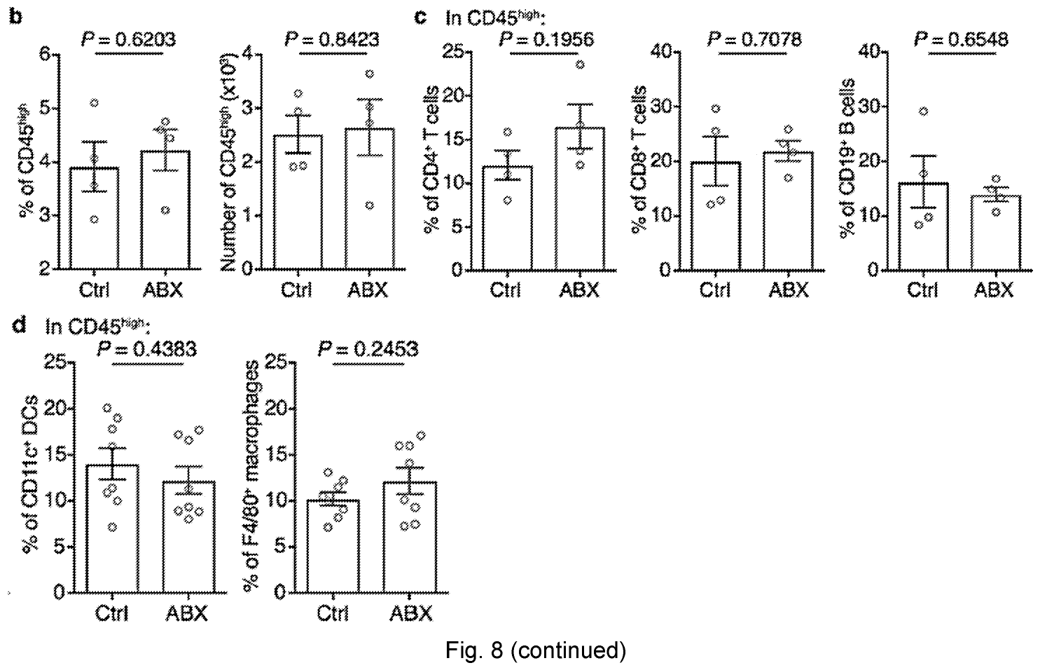

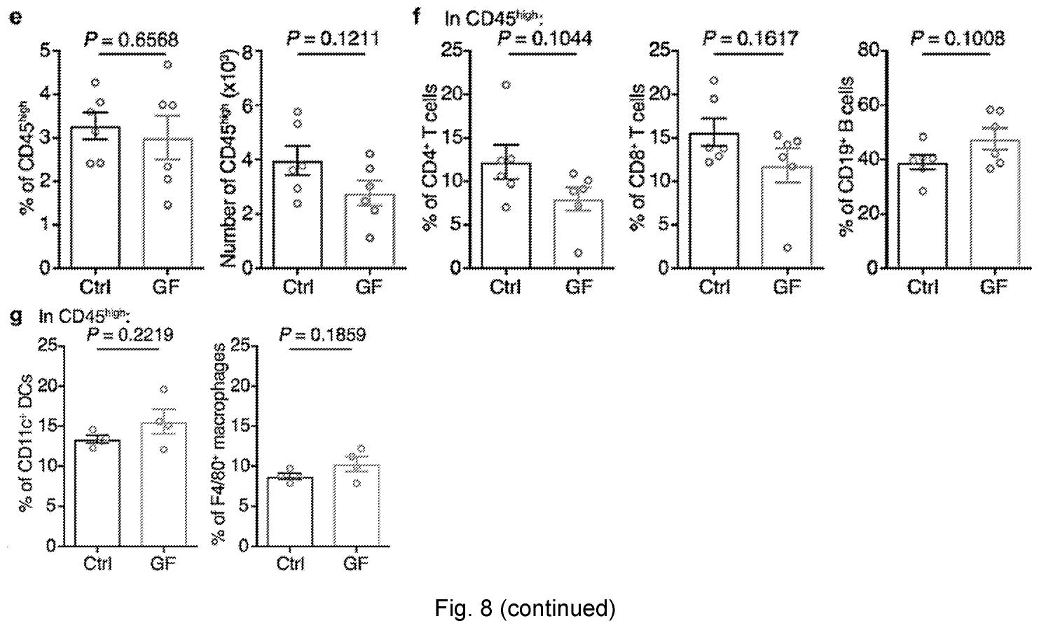

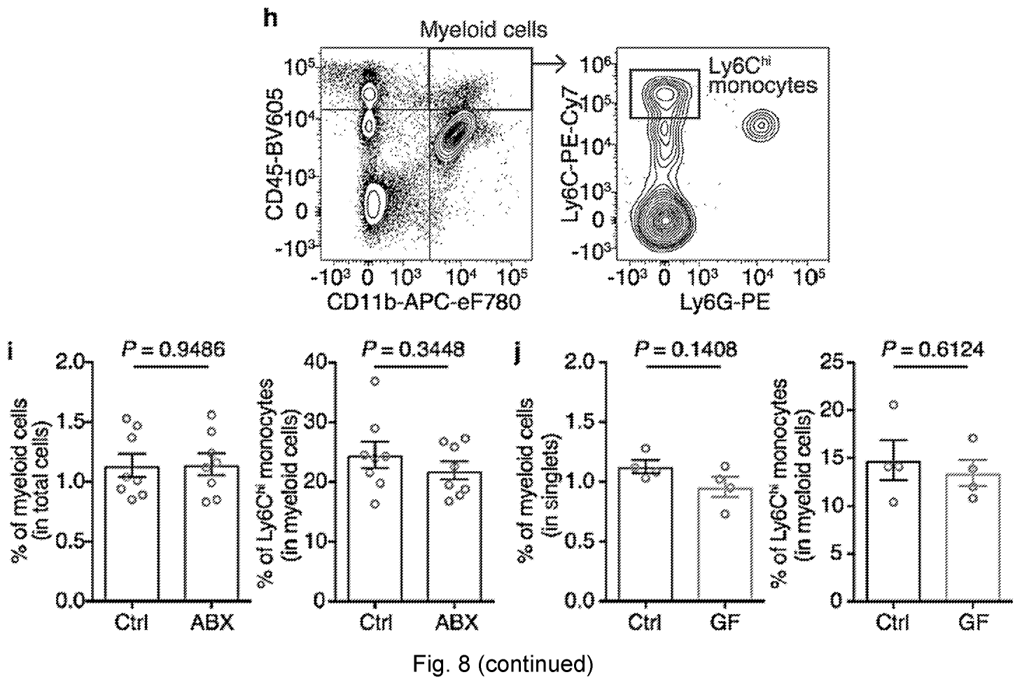

Further, we tested whether extinction learning deficits were associated with alterations in immune responses in the brain. Compared to Ctrl mice, ABX and GF mice showed no differences in the percentages and numbers of CD45^{high} leukocytes in the brain (FIG. 8a, b, e), and no differences in the percentages of CD4⁺ T cells, CD8⁺ T cells, CD19⁺ B cells, CD11c⁺ dendritic cells (DCs), F4/80⁺ macrophages and Ly6C^{hi} monocytes (FIG. 8c-d, f-j). Moreover, Rag1^{-/-} mice, which lack adaptive immune cells, exhibited normal extinction learning (FIG. 8k), while GF-Rag1^{-/-} mice exhibited deficits in extinction learning (FIG. 8l), indicating that the adaptive immune system is not required for extinction learning deficits in ABX and GF mice.

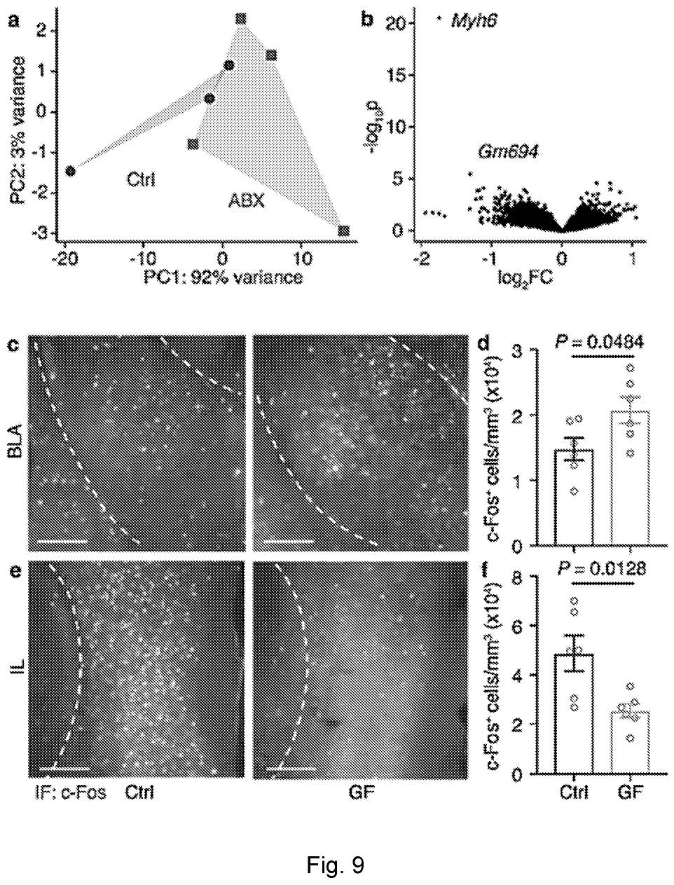

Given that deficits in extinction learning appear to occur independently of changes in the immune system, we sought to examine their neuroanatomical basis. We performed genome-wide transcriptional profiling of the medial prefrontal cortex (mPFC), an area of the brain known to play a critical role in extinction learning, from adult ABX and Ctrl mice. The mPFC tissue dissected in the absence of fear conditioning and extinction exhibited comparable transcriptomes (FIG. 9a, b). However, significant differences in the transcriptome of ABX and Ctrl mPFCs were revealed after extinction learning (FIG. 1f, g). Search tool for recurring instances of neighboring genes (STRING) analysis depicted networks of interactions of the differentially expressed genes (DEGs) between ABX and Ctrl samples (FIG. 1h-j); Kyoto encyclopedia of genes and genomes (KEGG) and gene ontology (GO) enrichment analyses revealed pathways that are associated with neuronal activity, synapse function, central nervous system maturation and the regulation of synaptic plasticity and postsynaptic dendritic spine development (FIG. 1k).

To test whether alterations in gene expression associated with these neuronal processes were associated with changes in neuronal activity, we examined neuronal activity in fear learning circuits by analyzing c-Fos expression in the basolateral amygdala (BLA), which is critical for encoding and storing conditioned fear memory, and in the infralimbic region (IL) of mPFC, which facilitates extinction learning. Compared to Ctrl mice, ABX and GF mice exhibited a higher density of c-Fos⁺ neurons in the BLA (FIG. 1l, m, FIG. 9c, d) and lower density of c-Fos⁺ neurons in the IL (FIG. 1n, o, FIG. 9e, f), which is consistent with their deficits in extinction learning.

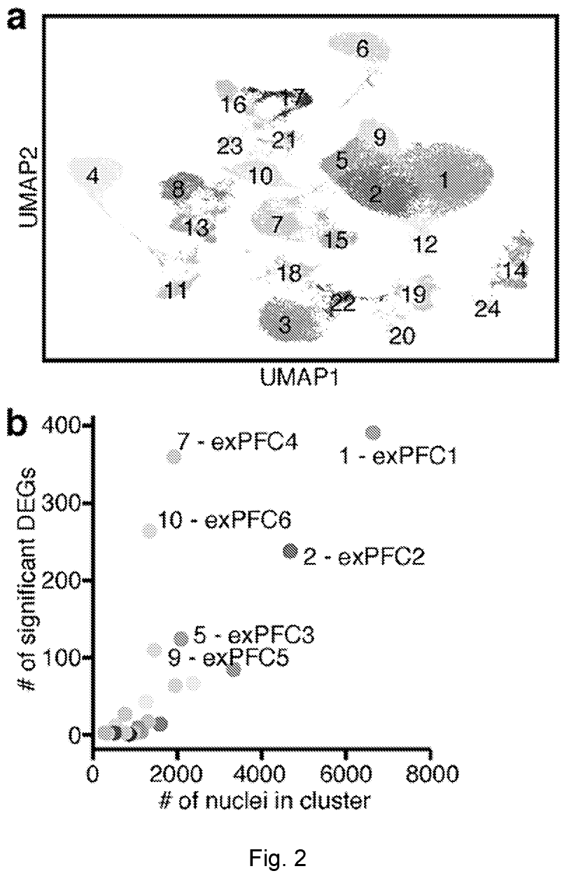

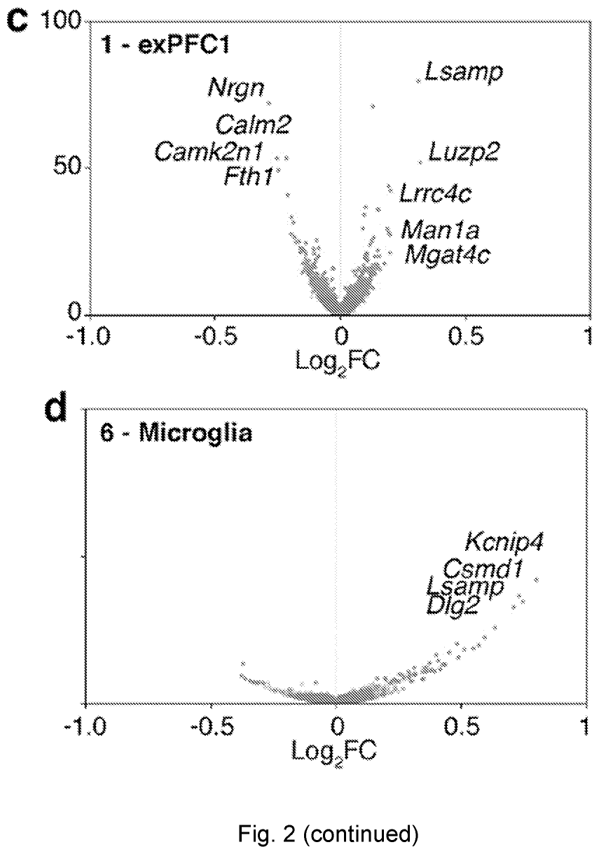

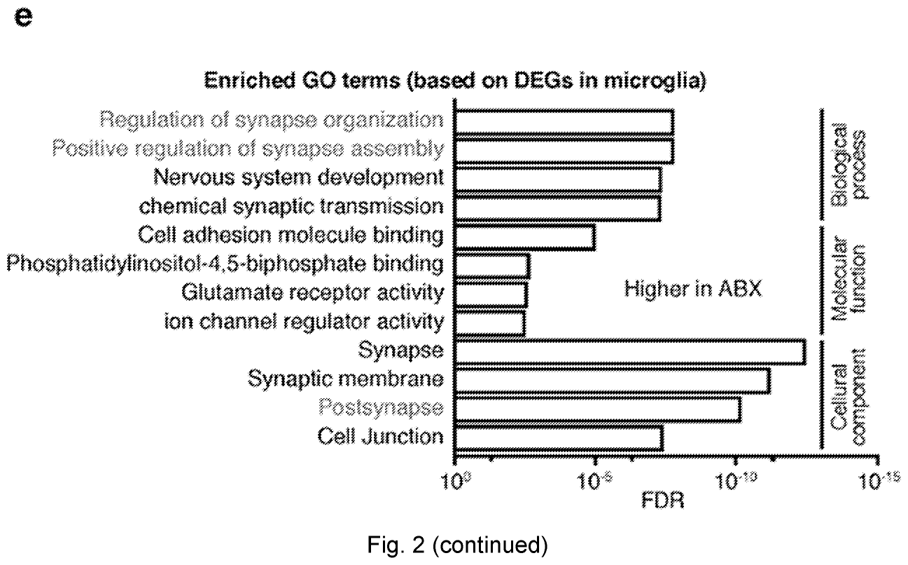

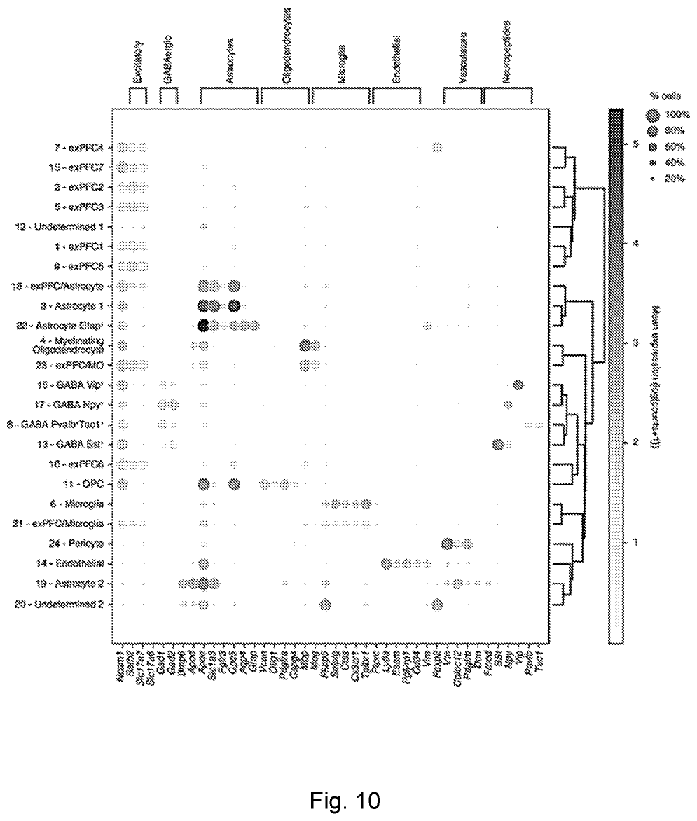

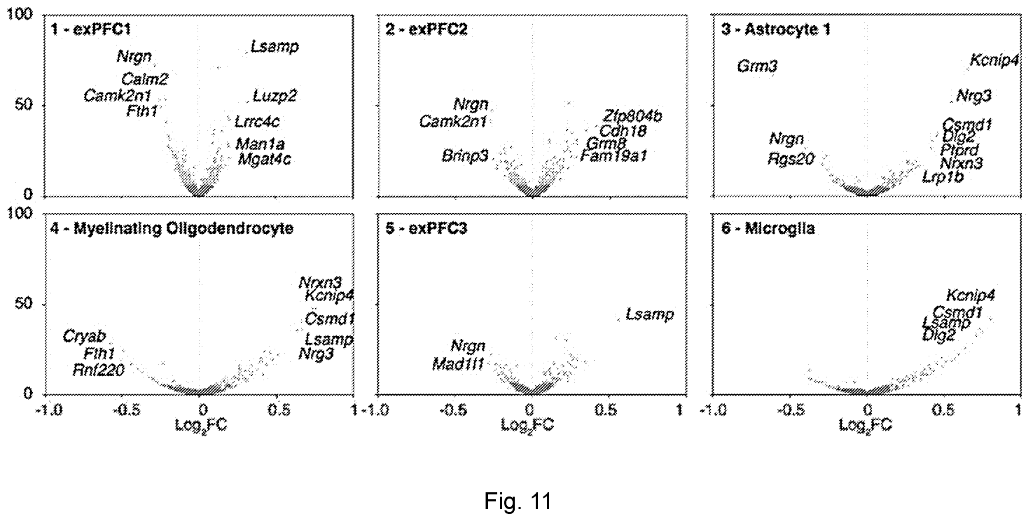

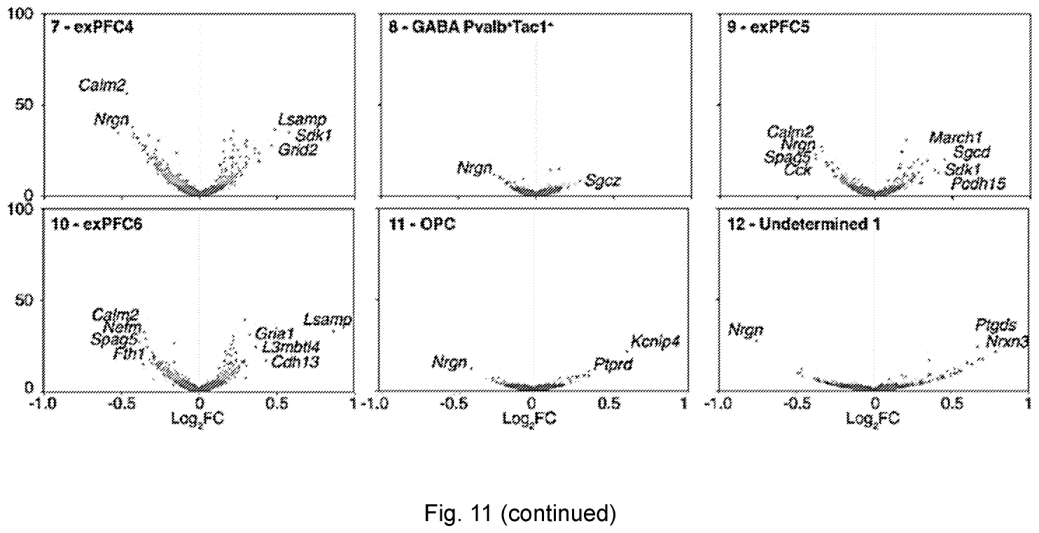

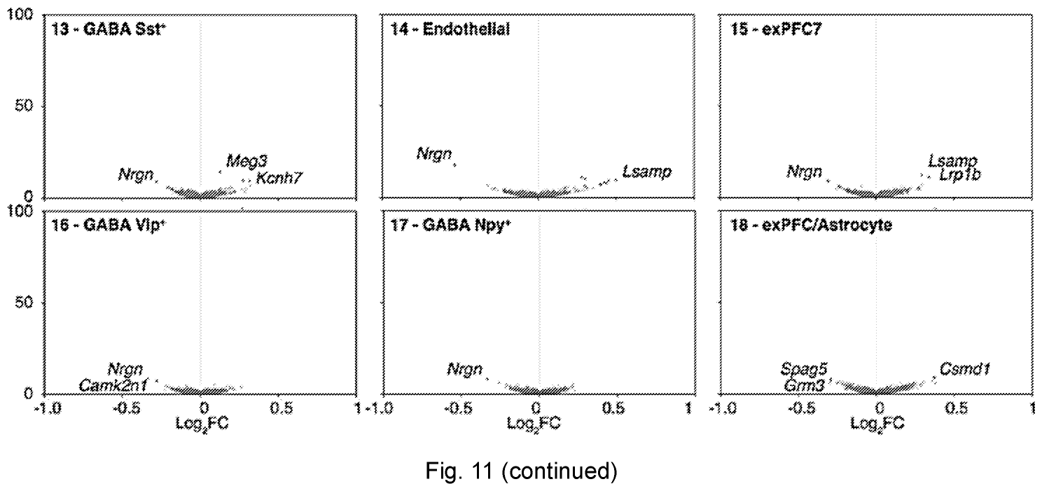

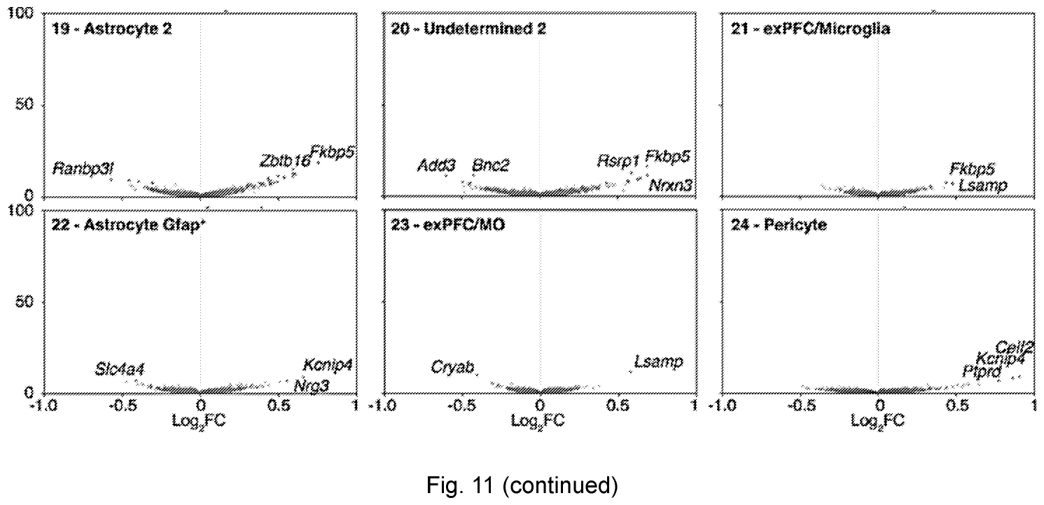

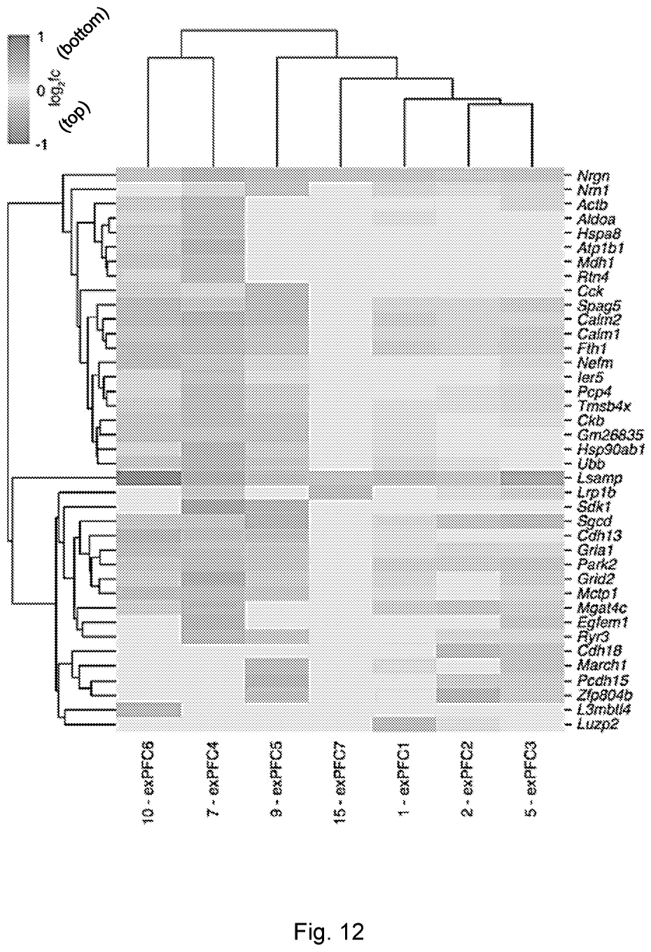

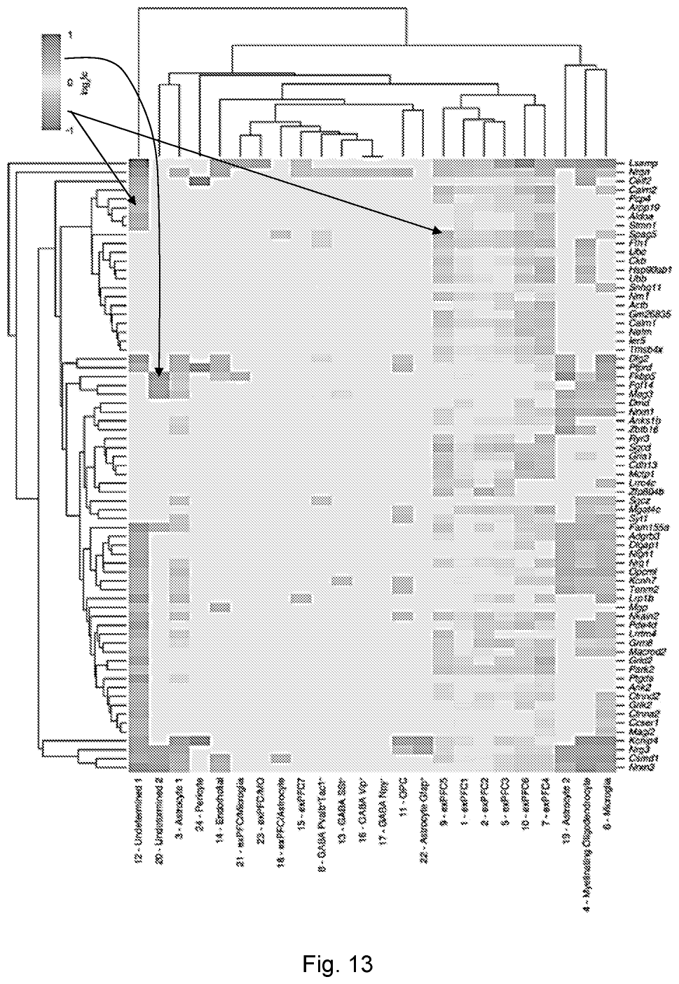

Altered excitatory neurons and microglia in the mPFC following alterations in microbiota. To define the cell subsets in the mPFC that contribute to the effect of the microbiota on extinction learning, we performed single nucleus RNA-seq (snRNA-seq) of mPFC samples dissected from ABX and Ctrl mice after extinction learning, and identified 24 cell clusters (FIG. 2a, FIG. 10). Changes in the microbiota were associated with significant changes in gene expression especially in excitatory neurons (FIG. 2b, c, FIG. 11), more so than in inhibitory neurons (including Pvalb⁺Tac1⁺, Sst⁺, Vip⁺ or Npy⁺ subset), oligodendrocyte progenitor cells (OPCs), endothelial cells and pericytes (FIG. 11). Both DEGs shared by excitatory neuronal subsets (FIG. 12), and DEGs shared by multiple cell types (FIG. 13) were linked to synapse-related pathways and calcium signaling pathways, which is consistent with our bulk RNA-seq data (FIG. 1k) and supports a model in which gene expression is altered in brain-resident cells, including specific cell populations such as excitatory neurons, following manipulation of the microbiota.

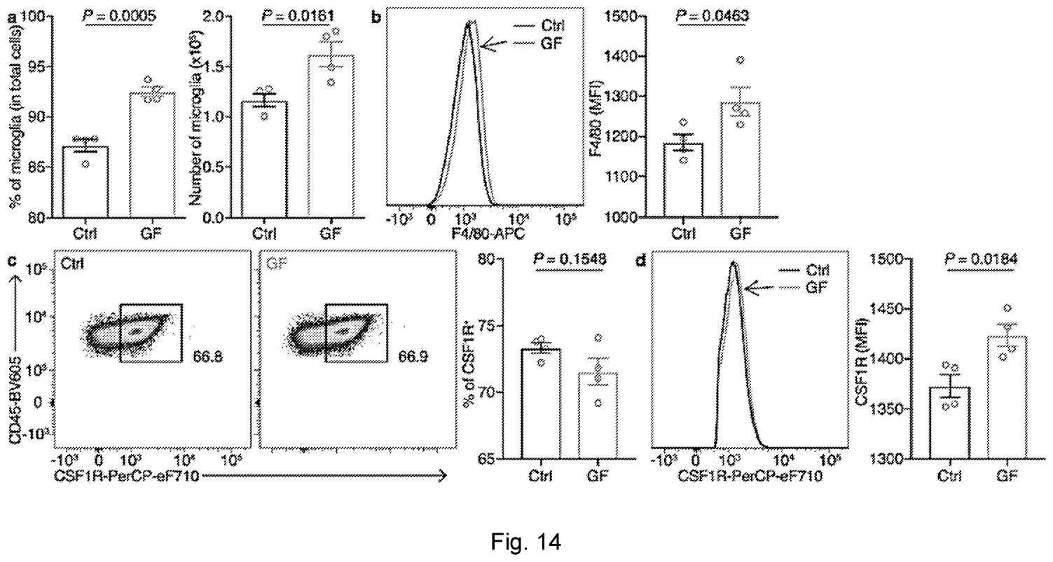

Given that microglia are important for maintaining neuronal function and brain health by dynamically regulating synaptic pruning and surveying their local microenvironment and have been reported to be affected by the microbiota, we further investigated the DEGs of microglia (FIG. 2d). The microglial DEGs were enriched in the pathways related to synapse organization and synapse assembly (FIG. 2e), suggesting that deliberate manipulation of the microbiota may alter microglia-mediated synaptic pruning. In addition, we found elevated percentages and numbers of microglia in GF mice, with elevated expression of CSF1R and F4/80 (FIG. 14a-d). The percentages and numbers of microglia in ABX mice were not changed, with no changes in CSF1R expression, but F4/80 expression was elevated (FIG. 14e-h). CSF1R and F4/80 are strongly developmentally regulated, with decreasing levels during maturation. Together, these data suggest that the microglia in GF and ABX mice exhibit an immature state reminiscent of developing juvenile microglia, which may in turn contribute to deficits in extinction learning by disrupting dendritic spine remodeling.

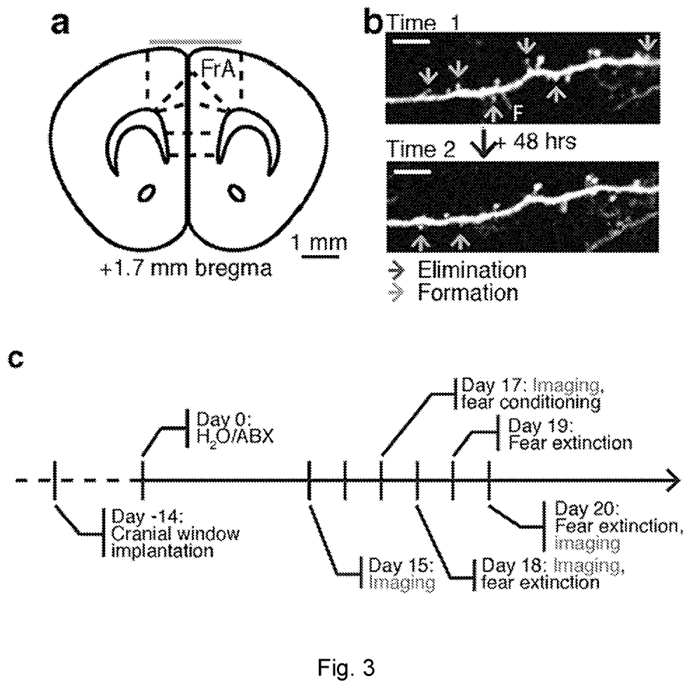

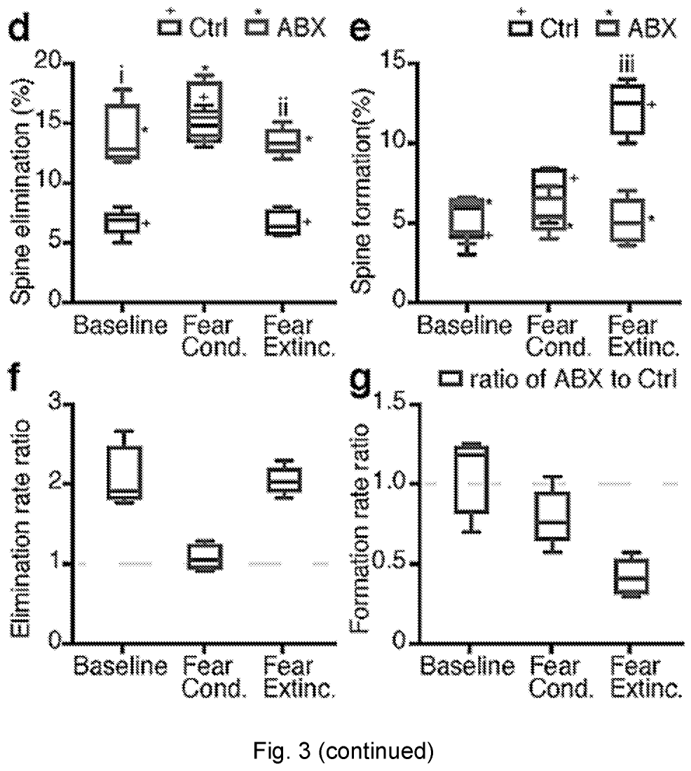

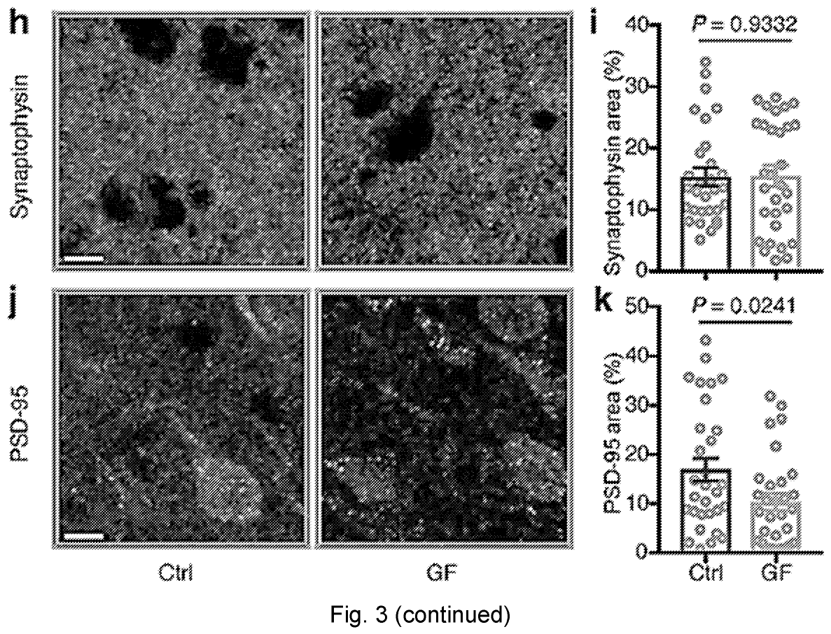

Microbiota alterations and defective extinction learning-related spine remodeling. Next, we employed two-photon laser scanning microscopy to directly quantify the remodeling of postsynaptic dendritic spines in the mPFC (FIG. 3a) during cued fear conditioning and extinction learning in transgenic Thy1/YFP reporter mice following manipulation of the microbiota in adulthood. Postsynaptic dendritic spines are membranous protrusions on neuronal dendrites that form primarily excitatory synapses with presynaptic axonal inputs and are dynamically remodeled during learning and development. We acquired images of the same dendritic spines during a baseline period and before and after fear conditioning and extinction learning (FIG. 3b, c). Compared to Ctrl mice, baseline spine elimination rates were significantly elevated in ABX mice (FIG. 3d, f), while baseline formation rates were unaffected (FIG. 3e, g). Cued fear conditioning and extinction learning had opposing effects on spine remodeling in Ctrl mice. Fear conditioning increased spine elimination rates in Ctrl mice (FIG. 3d), such that there was no significant difference in spine elimination or formation rates in the 24 hours after conditioning in ABX mice compared to Ctrl mice (FIG. 3d-g). In contrast, extinction learning-related spine remodeling was significantly altered in ABX mice. Extinction learning increased spine formation rates in Ctrl mice but not in ABX mice (FIG. 3e, g), and spine elimination rates remained persistently elevated in ABX mice relative to Ctrl mice (FIG. 3d, f).

Consistent with elevated spine elimination in ABX mice, we observed comparable expression of the pre-synaptic marker synaptophysin but lower expression of the post-synaptic marker PSD-95 in the mPFC of GF mice compared to Ctrl mice (FIG. 3h-k). In addition, the expression of Dlg4 in excitatory neuron subset 1 (exPFC1) was downregulated in ABX samples compared to Ctrl samples. Together, these data indicate that alterations in the composition of the microbiota are associated with deficits in learning-induced spine plasticity. Notably, plasma corticosterone levels were comparable in Ctrl, ABX and GF mice (FIG. 15a, b), indicating that HPA axis function may not be altered and is probably not driving microbiota-mediated changes in spine remodeling and fear extinction learning.

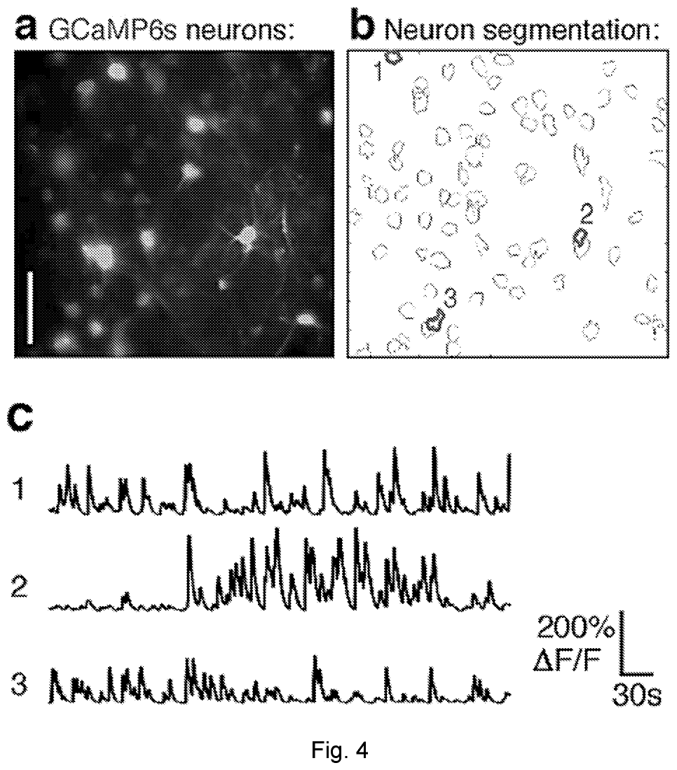

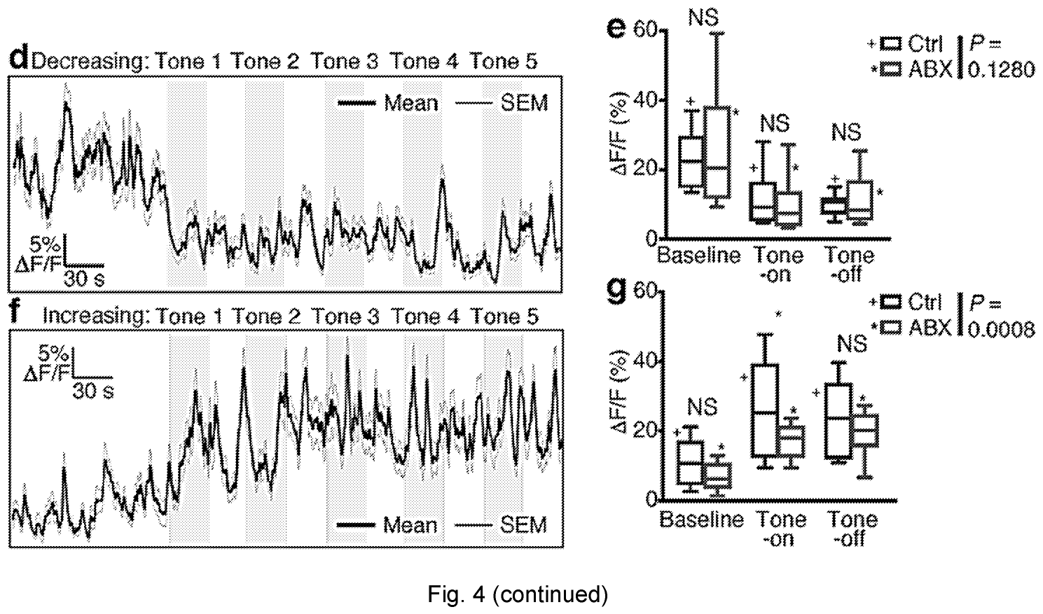

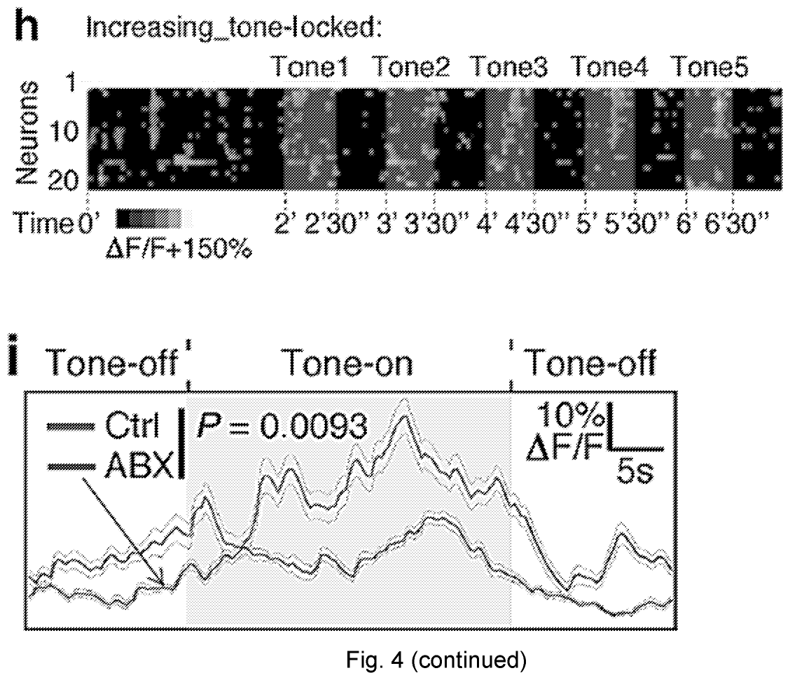

Defective tone-encoding neuronal ensembles in ABX mice. To investigate whether signals derived from the microbiota regulate learning-related neuronal activity in the mPFC, we used two-photon imaging and a genetically encoded calcium sensor (AAV5/hSyn/GCaMP6s) to quantify neuronal activity during extinction learning in Ctrl and ABX mice (FIG. 4a-c). We identified mPFC neurons that encoded the conditioned stimulus, and discovered two differentially responding functional cell types. In both Ctrl and ABX mice, one neuronal population (representing 13.5% of all cells) exhibited equivalently reduced activity during tone presentations (FIG. 4d, e). In contrast, a second population (representing 14.9% of all cells) displayed increased activity during tone presentations (FIG. 4f). Neuronal activity during tone presentations was modestly but significantly reduced in the latter cell population in ABX mice compared to Ctrl mice (FIG. 4g), consistent with their deficits in spine formation and behavior. Notably, 26.8% of these neurons also encoded the precise timing of the tones, exhibiting tone-locked activity that increased and decreased in response to the onset and offset of each tone, respectively (FIG. 4h). Again, tone-locked activity in these multicellular tone-sensitive ensembles was significantly reduced in ABX mice compared to Ctrl mice (FIG. 4i). In conjunction with the RNA-seq and dendritic spine remodeling analyses, these data indicate that dysbiosis of the gut microbiota disrupts learning-related spine formation and interferes with the emergence of multicellular tone-encoding ensembles.

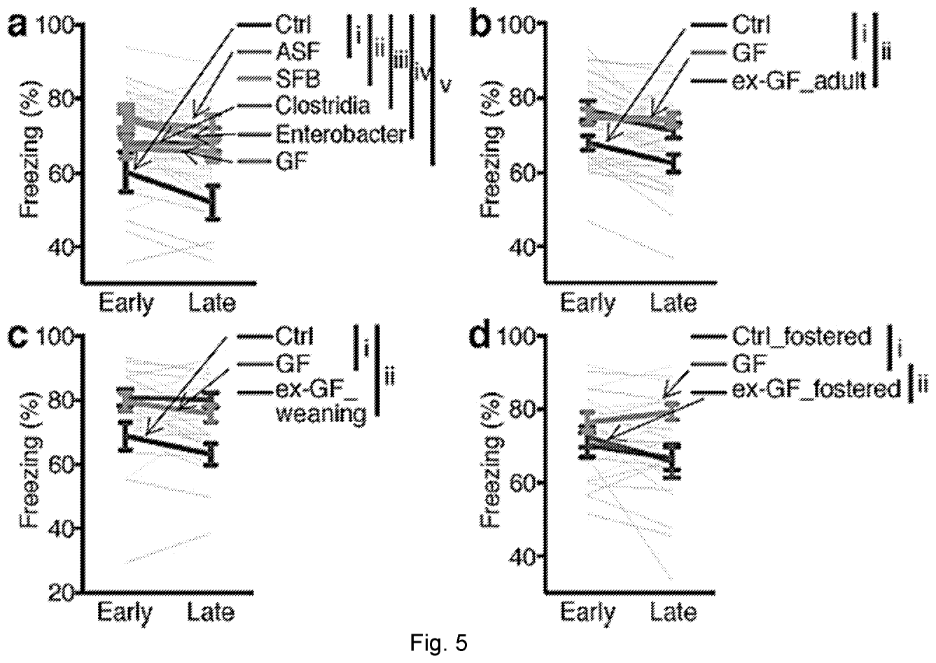

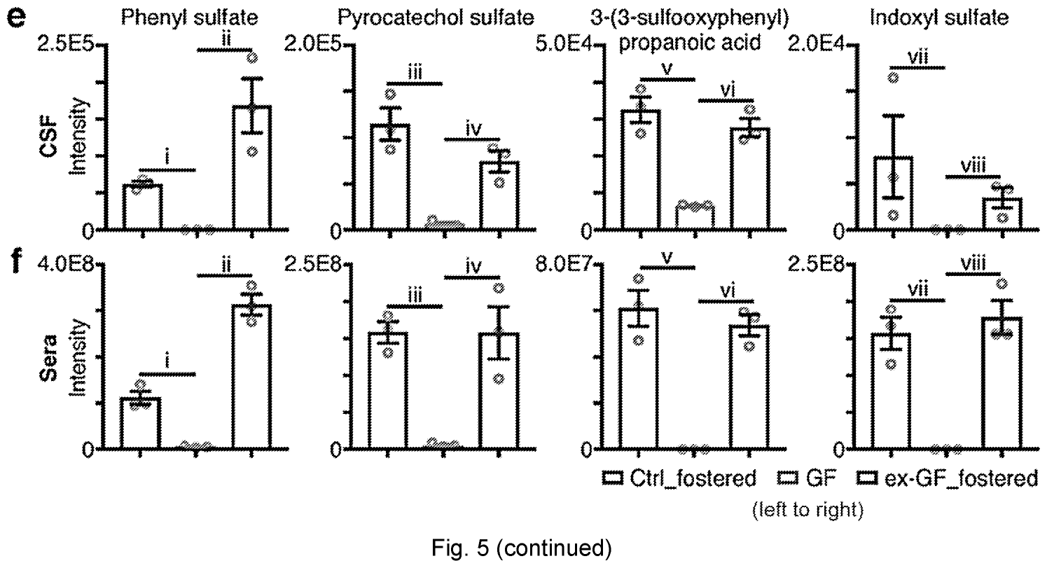

A diverse microbiota is required to restore extinction learning. To test whether the extinction learning deficits caused by altered microbiota can be rescued by colonization with defined individual microbes or consortia of microbes, we performed fear conditioning and extinction learning in gnotobiotic mice colonized with bacteria that are known to influence other physiologic processes. Notably, following colonization with segmented filamentous bacterium (SFB), Clostridia spp., Enterobacter spp., or altered Schaedler flora (ASF), these gnotobiotic mice still exhibited impaired extinction learning compared to Ctrl mice (FIG. 5a), suggesting that a more diverse microbiota is required for normal extinction learning and fear extinction behavior.

To investigate whether the extinction learning deficits caused by altered microbiota are reversible, we re-colonized previously germ-free (ex-GF) mice with a complete microbiota from healthy Ctrl mice at various developmental time points. Both ex-GF mice colonized when they were adults and ex-GF mice colonized at weaning age still displayed impaired fear extinction compared to Ctrl mice (FIG. 5b, c), indicating that extinction learning deficits were not reversible in GF mice after weaning. However, when ex-GF mice were colonized immediately following birth via fostering to microbiota-replete specific pathogen-free surrogate mothers (ex-GF_fostered mice), they exhibited a restoration of normal fear extinction behavior comparable to Ctrl_fostered mice (FIG. 5d), indicating that extinction learning and learning-related plasticity require microbiota-derived signals during a critical developmental period prior to weaning. Lack of the microbiota in the neonatal period, no matter whether microbially-colonized or not after weaning, renders deficits in fear extinction learning in adulthood. While ex-GF_fostered mice restored fear extinction, we found no significant shift in the transcriptome of the mPFC of the ex-GF_fostered mice or Ctrl_fostered mice, compared to GF mice after the single-session fear extinction (data not shown). This could reflect the shorter extinction model employed in GF mice that may induce smaller transcriptional changes than the 3-day/session fear extinction model employed in ABX mice. Alternatively, the lack of transcriptional changes in the GF fostering studies could indicate other processes such as post-translational or epigenomic modifications.

Altered metabolites in the brain are associated with fear extinction. Next, we sought to examine whether changes in neuronal development and fear extinction learning were associated with alterations in microbiota-derived metabolites. We employed untargeted comparative metabolomics of cerebrospinal fluid (CSF), serum and fecal samples from adult GF mice, Ctrl_fostered mice and ex-GF_fostered mice using high-resolution liquid chromatography-mass spectrometry (LC-MS). Using the xcms platform for comparative analysis of the MS data sets, we identified four metabolites—phenyl sulfate, pyrocatechol sulfate, 3-(3-sulfooxyphenyl)propanoic acid (all phenolic compounds) and indoxyl sulfate (FIG. 15c)—that were significantly decreased in CSF, serum and fecal samples of GF mice compared to Ctrl_fostered mice, and were recovered in ex-GF_fostered mice (FIG. 5e, f, FIG. 15d). Downregulation of the same four metabolites was also detected in comparisons of GF vs. Ctrl CSF samples (FIG. 15e), as well as in comparisons of ABX vs. Ctrl samples (data not shown).

Methods. Experiments are performed in triplicate and employ rigorous statistical methods, including non-parametric statistical testing (Wilcoxon rank sum, Kruskal Wallis ANOVA) when relevant. Equal numbers of male and female mice (7-12 weeks old, 8 per group) are typically included in all studies. Experiments are performed in triplicate and employ rigorous statistical methods, including non-parametric statistical testing (Wilcoxon rank sum, Kruskal Wallis ANOVA) when relevant. Equal numbers of male and female mice (7-12 weeks old, 8 per group) are typically included in all studies. All mice in all experiments are randomized to conditions or treatments using a random number generator, and experimenters and data analysts are blind to group assignment throughout when possible. Mice are housed in SPF conditions with standard temperature, humidity, and light/dark cycles. Co-housed littermate control mice are used to minimize cage effects and drift in the microbiota composition. Both male and female mice were used in studies.

Mice. C57BL/6J (Jax 664), Rag1^{-/-} (Jax 2216), Thy1-YFP-H (Jax 3782) and BALB/c (Jax 651) mice were purchased from The Jackson Laboratory and bred in-house. Male mice were used at 7-16 weeks of age. In individual experiments, all animals were age-matched. All mice were maintained under specific pathogen-free (SPF) conditions on a 12-hour light/dark cycle, and provided food and water ad libitum. Germ-free C57BL/6 mice and gnotobiotic mice were maintained at Weill Cornell Medical College, New York. All mouse experiments were approved by, and performed in accordance with, the Institutional Animal Care and Use Committee guidelines at Weill Cornell Medicine.

Antibiotic treatment. Mice were provided autoclaved drinking water supplemented with a cocktail of broad-spectrum antibiotics: ampicillin (0.5 mg/mL, Santa Cruz), gentamicin (0.5 mg/mL, Gemini Bio-Products), metronidazole (0.5 mg/mL Sigma), neomycin (0.5 mg/mL, Sigma), vancomycin (0.25 mg/mL, Chem-Impex International), and saccharin (4 mg/mL, Sweet'N Low, Cumberland Packing Corp.). Sweet'N Low was added to make the antibiotic cocktail more palatable. Antibiotic treatment was started 2 weeks prior to the experiments and continued for the duration of the experiments. Following ABX treatment mice exhibited no significant differences in weight gain, food or water intake (measured by Promethion metabolic cages) and perception of pain.

Fear conditioning and extinction assays. Fear conditioning and extinction assays were performed as follows. For fear conditioning, mice were placed in shock-chambers (Coulbourn Instruments), which were scented with 0.1% peppermint in 70% EtOH. After 2 mins of habituation, mice were fear conditioned with 3 tone-shock pairings consisting of a 30 second (5 kHz, 70 dB) tone (CS) that co-terminated with a 1 second (0.7 mA) foot shock (US). Interatrial intervals (ITIs) between each tone-shock pairing were 30 seconds. After the final tone-shock pairing, mice remained in the conditioning chambers for 1 min before being returned to their home cages.

For the classical 3-day/session fear extinction, 24 hours after fear conditioning, mice were placed in extinction chambers (different shape from the conditioning chambers), which were scented with 0.1% lemon in 70% EtOH. After 2 mins of habituation, mice were exposed to 5 presentations of the tone (CS) in the absence of the shock (US). Each tone lasted for 30 seconds with an ITI of 30 seconds. After the final tone presentation, mice remained in the extinction chambers for 1 min before being returned to their home cages. Fear extinction sessions were repeated daily for three days.

For single-session 30-tone fear extinction, 20 mins after fear conditioning, mice were placed in extinction chambers. After 2 mins of habituation, mice were exposed to 30 presentations of the tone (CS) in the absence of the shock (US). Each tone lasted for 30 seconds with an ITI of 30 seconds. Extinction trials were binned into early and late sessions, with the early session representing the average of trials 1-15, and late trials representing the average of trials 16-30.

Experiments were controlled by Graphic State software (Coulbourn instruments). Mice were video recorded for subsequent analysis.

Fear behavior. Mouse freezing behavior was scored automatically using previously validated MATLAB code for automated phenotyping of mouse behavior. Percent time spent freezing was calculated by dividing the amount of time spent freezing during the 30-second tone presentations by the duration of the tone.

Immunofluorescence staining. Brain sections were prepared and stained for c-Fos, synaptophysin or PSD-95 expression. All steps were carried out at room temperature unless otherwise specified. 90 minutes after fear extinction session 3, mice were anesthetized by intraperitoneal injection of Euthasol and perfused with PBS followed by 4% paraformaldehyde. Brains were harvested, fixed in 4% paraformaldehyde overnight, and dehydrated in 30% sucrose at 4° C. Coronal sections (40 μm) were cut by using sliding microtome frozen by powdered dry ice. 6 sets of serial sections were collected in Eppendorf tubes each containing 2 mL cryoprotectant (30% glycerol and 30% ethylene glycol in 0.1 M sodium phosphate, pH 7.4) and stored at −20° C. Free-floating serial sections (take one every third) were washed in TBS, incubated for 30 min in blocking buffer (4% normal horse serum, 1% BSA and 0.2% Triton X-100 in TBS) and incubated overnight at 4° C. with rabbit anti-c-Fos primary antibody (sc-52, Santa Cruz), or mouse anti-synaptophysin (SAB4200544, Sigma-Aldrich) or PSD-95 (7E3-1B8, Sigma-Aldrich) diluted 1:1,000 in the blocking buffer. Sections were then washed in TBS and incubated for 2 hours with Alexa Fluor 555-labelled donkey anti-rabbit or anti-mouse antibody (Invitrogen) diluted 1:500 in TBS with 0.2% Triton X-100. Sections were again washed, mounted on chromalum/gelatin-coated slides and air-dried for 2 hours in dark. Slides were cover-slipped by water-soluble glycerol-based mounting medium containing DAPI and sealed with nail polish.

Estimation of cell densities of c-Fos⁺ neurons in BLA and IL was performed with StereoInvestigator 9.0 (MicroBrightField). Briefly, serial sections (every third section, 120 μm) were numbered by rostra-caudal order, and contours of BLA and IL were traced by referring to the Allen Brain Atlas (Allen Institute). All cells across all sections per animal were counted. Individual cell density was calculated for each mouse by dividing the total sampled cell numbers by the total volume of the region.

For synaptophysin and PSD-95 images, confocal microscopy was performed with a Zeiss LSM 880 Laser Scanning Confocal Microscope using 63× oil immersion lens. Images were acquired with 2× digital zoom. Image stacks were 5 μm in thickness with z-step size of 0.5 and were analyzed using the ImageJ software (rsbweb.nih.gov/ij).

Intracranial window implantation. Mice were anesthetized by isoflurane (induction, 5%; maintenance, 1%-2%) and administered dexamethasone (1 mg/kg, i.p.) to reduce brain swelling and metacam (2 mg/kg, i.p.) as a prophylactic analgesic. Scalp fur was trimmed, and the skull surface was exposed with a midline scalp incision. Bupivacaine (0.05 mL, 5 mg/ml) was administered topically as a second prophylactic analgesic. A circular titanium head plate was positioned over the region to be imaged (1.7 mm anterior to the bregma suture and centered over the midline) using dental cement (Metabond). A high-speed dental drill (Model EXL-M40, Osada Inc) and 0.5 mm burr were used to open a small (˜4 mm) craniotomy. A 3 mm round coverslip (Warner Instruments) was lowered through the craniotomy to rest on top of the brain using a digital micromanipulator. The window was then fixed to the skull using veterinary adhesives (first Vetbond, then Metabond).

Viral injection. AAV5/hSyn/GCaMP6s was obtained from the UPenn Vector Core. Viral injection surgeries were performed with mice (8-10 weeks of age) under isoflurane anesthesia (induction, 5%; maintenance, 1%-2%) with regular monitoring for stable respiratory rate and absent tail pinch response. The scalp was shaved, and mice were fixed in a stereotactic frame (Kopf Instruments) with non-rupturing ear bars. A heating pad was used to prevent hypothermia. A midline incision was made to expose the skull and bupivacaine was applied onto the skull for local anesthesia. Virus injections (1000 nL) were delivered with a 10 μL Hamilton syringe and 33-gauge beveled needle, injected at 100 nL/min using an injection pump (World Precision Instruments). Injection coordinates relative to Bregma were: 1.7 mm anterior, 0.4 mm lateral, and 1.3 mm ventral. Following injection, the injection needle was held at the injection site for 2 mins then slowly withdrawn. The skin was then closed by Vetbond (3M Company) and the mice recovered on a heating pad before being returned to their home cages.

Transcranial two-photon imaging. Dendritic spine imaging was conducted. Briefly, image stacks of dendritic segments were acquired using a two-photon laser scanning microscope (Olympus RS) equipped with a scanning galvanometer and a Spectra-Physics Mai Tai DeepSee laser tuned to 920 nm, and a 25×, long working distance water-immersion microscope objective (NA=1.05, Olympus). Fluorescence was detected through gallium arsenide phosphide (GaAsP) photomultiplier tubes (PMTs) using the Fluoview acquisition software (Olympus), and images were collected in the green channel using an F30FGR bandpass filter (Semrock). All imaging experiments began by obtaining a low-magnification z stack (no digital zoom) to aid in relocating the same sites repeatedly over time, in conjunction with vascular landmarks and the contours of the prism. For spine imaging experiments, we acquired z stacks (512×512 pixels, 2-μs pixel dwell time, 0.75-1 μm step size) with 3× digital zoom through up to 250 μm of tissue in z. Spine imaging experiments occurred under KX anesthesia (ketamine 100 mg/mL and xylazine 10 mg/mL, at dosages of 0.1 mL/10 g body weight). For calcium imaging experiments, we acquired time-lapse images (512×512 pixels, 3 frames per second, ˜1,450 frames) spanning an area of mPFC measuring approximately 508 μm by 508 All calcium imaging experiments occurred in awake mice. For repeated imaging over intervals of days, the procedure above was repeated, and the region to be imaged was identified by referring to vascular landmarks and the contours of the cranial window.

Spine imaging analysis. Spine remodeling dynamics were quantified. Image stacks were analyzed using the ImageJ software. Raters blinded to experimental condition compared pairs of images of the same dendritic segment and identified stable spines (present in image 1 and 2), eliminated spines (present in image 1 but not in image 2) and formed spines (present in image 2 but not in image 1), each quantified as a percent of the total number of spines identified in the initial image. Filopodia were defined as dendritic protrusions with a length exceeding three times their maximum width and were excluded from spine remodeling analyses.

Calcium imaging analysis. Preprocessing. We used standard, validated procedures for preprocessing and analyzing calcium imaging time series data. X-Y motion artifacts were corrected using the ImageJ plugin. Image time series were segmented into individual cells using custom MATLAB scripts based on an established sorting algorithm combining independent components analysis and image segmentation based on threshold intensity, variance, and skewness in the x-y motion corrected data set. Image segmentation results were manually inspected for quality control. Fluorescence signal time series (ΔF/F: change in fluorescence divided by baseline fluorescence) were calculated for each individual neuronal segment: a 40-s sliding window was used to calculate the baseline fluorescence for each cell, accounting for both differences in GCaMP expression and de-trending for slow time-scale changes in fluorescence.

Analysis. First, we tested for cells exhibiting tone-sensitive activity, using repeated measures ANOVA to identify cells with a statistically significant increase (FIG. 4f) or decrease (FIG. 4d) in activity during tone presentations, compared to their activity during a two-minute pre-tone baseline period. To estimate statistical significance while accounting for autocorrelation in calcium transient time series and correcting for multiple comparisons, we repeated this analysis 10,000 times for each cell after shuffling the timing of the baseline period and the timing of the tone onsets and selected a P value threshold to limit the false discovery rate to less than 5%. Next, to test for group (ABX vs. Ctrl) effects on activity in each of these cell populations (FIG. 4e, g), we used a 2-factor (time, group) repeated measures ANOVA and post-hoc linear contrasts to test for between-group differences in activity during each task epoch (baseline, tone on, tone off). Finally, to test for cells that also encoded the precise timing of the tones, exhibiting tone-locked activity that increased or decreased in response to the onset of each tone, we used a procedure analogous to the one described above, using repeated measures ANOVA to test for changes in activity in the tone on vs. tone off epochs; estimating statistical significance in shuffled data as above; and testing for group effects on activity using a 2-factor (time, group) repeated measures ANOVA (FIG. 4h, i).

RNA sequencing. Mouse mPFC was dissected by referring to the Allen Brain Atlas. Coordinates relative to bregma are: 1.3 to 2.8 mm anterior, −1 to 1 mm lateral, and 0 to 1 mm ventral. RNA was extracted using Trizol (Invitrogen) and chloroform and further purified using the RNAeasy mini spin columns (Qiagen). RNA-seq libraries were prepared and sequenced by the Epigenomics Core at Weill Cornell Medicine on an Illumina HiSeq 2500, producing 50 bp single-end reads. Sequenced reads were demultiplexed using CASAVA v1.8.2 and adapters trimmed using FLEXBAR v2.4.

RNA-seq analysis. Sequenced reads were aligned to the mouse genome GRCm38/mm10 using STAR v2.3.0. Reads counts at the gene level were calculated using Rsubread. Normalization for library size and differential expression analysis were performed using DESeq2 v1.18. Only genes with at least 10 raw reads in each sample were tested for differential expression. Nonparametric multivariate analysis of variance (PERMANOVA) was used to test whether antibiotics treatment accounted for a significant portion of the variance in gene expression post fear extinction. Specifically, expression of the 500 genes with the highest variance (after applying the variance stabilization transformation of DESeq2) was analyzed using the adonis function of the vegan R package (CRAN.R-project.org/package=vegan) using the Euclidean metric and 20,000 permutations. Differentially expressed genes were used for Gene Ontology (GO) enrichment analysis, Kyoto Encyclopedia of Genes and Genomes (KEGG) analysis and Search Tool for Recurring Instances of Neighboring Genes (STRING) analysis.

Brain-resident immune cell isolation and flow cytometry. Brain-resident immune cells were isolated using Percoll gradients. Mice were anesthetized and perfused with ice-cold HBSS. Brains were harvested, homogenized, resuspended with 30% Percoll, and layered on top of 70% Percoll. After centrifugation (500×g, 30 min), immune cells gathered in the 30%-70% interphase.

For flow cytometric analyses, cells were washed, incubated with purified anti-mouse CD16/CD32 (clone 93, Biolegend) to block the Fc receptors, and then stained with anti-CD45 (clone 30-F11, Biolegend), anti-CD4 (clone RM4-5, eBioscience), anti-CD8a (clone 53-6.7, BD Biosciences), anti-CD19 (clone 1D3, eBioscience), anti-CD11b (clone M1/70, eBioscience), anti-CD11c (clone N418, eBioscience), anti-F4/80 (clone BM8, eBioscience), anti-Ly6G (clone 1A8-Ly6g, eBioscience), anti-Ly6C (clone HK1.4, eBioscience) and anti-CSF1R (clone AFS98, eBioscience). Data were collected on a LSRFortessa cytometer (BD Biosciences) and analyzed with FlowJo software (Tree Star Inc.). Dead cells were excluded from analyses based on LIVE/DEAD™ Fixable Aqua dead cell staining (Invitrogen). Non-singlet events were excluded from analyses based on the side scatter height (SSC-H) versus side scatter width (SSC-W), and then the forward scatter height (FSC-H) versus forward scatter width (FSC-W) characteristics.

Microbial colonization. For ex-GF mice colonized when they were adults (ex-GF_adult), dirty bedding from SPF mice were added into GF cages of 8 weeks old GF mice two weeks before the fear conditioning and extinction assay. For ex-GF mice colonized when they were weaned (ex-GF_weaning), 3 weeks old GF mice were co-housed with 3 weeks old SPF mice until 8 weeks old and subjected to the fear conditioning and extinction assay.

Fostering of pups. C57BL/6 GF and Ctrl SPF new born pups were fostered by BALB/c mothers until weaning.

16S qPCR. DNA was isolated from fecal samples of Ctrl and ABX mice using the DNeasy PowerSoil kit (Qiagen). Equal amounts of purified fecal DNA (4 ng per reaction) were added to qPCR reactions with universal 16S primers using SYBR green chemistry (UniF340: 5′-ACTCCTACGGGAGGCAGCAGT-3′ (SEQ ID NO:1); UniR514: 5′-ATTACCGCGGCTGCTGGC-3′ (SEQ ID NO:2)). 16S DNA levels in each sample were normalized to the average of the Ctrl mice group.

16S amplicon sequencing and analysis. 16S rRNA gene sequencing methods were adapted from the methods developed for the NIH-Human Microbiome Project. Briefly, bacterial genomic DNA was extracted using MO BIO PowerSoil DNA Isolation Kit (MO BIO Laboratories). The 16S rDNA V4 region was amplified by PCR and sequenced in the MiSeq platform (Illumina) using the 2×250 bp paired-end protocol. Raw reads were processed and clustered into operational taxonomic units (OTUs) using USEARCH version 11. Specifically, reads were demultiplexed and read pairs merged, with a maximum of 5 mismatching bases in the overlap region, as well as a minimum sequence agreement of 80%. PhiX contaminant sequences were removed, and merged sequences were filtered according to FASTQ quality scores using a maximum expected error number of 0.1. Filtered sequences were clustered into OTUs at a 97% identity threshold using the USEARCH cluster_otus command with default settings. Merged reads (unfiltered) were mapped to the OTU representative sequences, generating an OTU table. Taxonomic classification of OTU representative sequences was performed with the USEARCH SINTAX command with a confidence score of 0.8, using version 16 of the RDP 16S training set. Diversity estimation and PCoA ordination were performed using the phyloseq R package after subsampling the OTU table to even depth.

Single nucleus RNA-seq. Nuclei were extracted from four frozen mPFC samples (two from ABX mice, two from Ctrl mice) with a glass dounce tissue grinder set (Millipore Sigma #D8938) and Nuclei EZ Prep (Millipore Sigma #NUC101-1kt). Each sample was dounced with pestles A and B (24× each) in 2 mL of EZ prep buffer, washed with 5 mL EZ prep, and resuspended in 1 mL resuspension buffer (1×PBS, 0.1% BSA, 25 U/mL recombinant RNase inhibitor, Takara 2313B). Single nucleus suspensions were strained through a 35 μm cell strainer (Corning 352235), visually inspected under a microscope, and loaded onto 3′ library chips as per the manufacturer's protocol for the Chromium Single Cell 3′ Library & Gel Bead kit (v3) (10× Genomics 1000092). For each sample, an input of 11,000 nuclei was added to each channel. Libraries were sequenced at a mean depth of 21,714 reads per nucleus on a HiSeqX.

Single nucleus RNA-seq data processing. Demultiplexed FASTQ files were generated using Cell Ranger v2.0. Reads were aligned to the mm10 mouse transcriptome containing pre-mRNA annotations, to generate raw gene expression matrices (nuclei by genes). Expression matrices across all four samples were merged and loaded into Scanpy (version 1.4.0). Genes found in less than 3 nuclei were filtered out. Nuclei were filtered out using the following criteria: less than 600 genes (likely empty droplets), more than 5,000 genes (likely doublets), >2% of reads mapping to mitochondrial genes, >0.1% of reads mapping to caspase genes to remove apoptotic cells. The resulting filtered matrix consisted of 38,649 nuclei and 22,451 genes. The filtered gene expression matrix was normalized within each nucleus, resulting in a filtered, nuclei-normalized matrix X, then log-normalized by calculating ln (X+1). Before selecting variable genes, we masked genes that either contain highly repetitive regions in intronic regions that result in inflated read counts (PISD, Mylip, Gm17660) or highly expressed lncRNAs that affect within-nuclei normalization (Gm28928, Malat1). We selected 1,535 highly variable genes using the highly variable genes module in scanpy (min_mean=0.1, max_mean=3, min_disp=0.8) for clustering analysis.

snRNA-seq data clustering. We first regressed out the number of UMIs and the number of genes. Each gene was then scaled to unit variance. We then conducted dimensionality reduction via principal component analysis (PCA) using the ARPACK SVD solver in scanpy, computed the k-nearest neighbor (k-NN) graph with PCs 1 to 40 and k=30 nearest neighbors. Clusters were determined with unsupervised clustering using the Louvain algorithm and resulted in 24 clusters. Differential expression analysis was conducted to find the top 100 genes induced in the nuclei each cluster relative to all other nuclei with the rank_genes_groups module in scanpy using logistic regression. We annotated clusters post hoc based on known marker genes among the top-100 induced genes. For visualization, we embedded the profiles with UMAP (uniform manifold approximation and projection).

Cell-type specific differential expression analysis. To find differentially expressed genes between ABX vs. Ctrl mice for each cell type, we used statsmodels in python to implement a mixed linear model for each cluster c. Specifically, we used the regression Y_{i,c}˜T+N+(1|B), where Y_{i,c} is the ln (X+1) expression vector for gene i across all nuclei in cluster c, T is a binary variable reflecting membership of the nucleus in either ABX or Ctrl sample, N is the number of genes detected in each nucleus, and B is a categorical variable denoting the 10× channel used for each sample to control for batch effects. We used a Bonferroni-corrected p-value of 10⁻⁷ as the cutoff for significance. For plotting, we used differentially expressed genes that had a minimum log₂ (fold change) of 0.31 (absolute fold change of 1.24) in either direction, and independently found to be significant in at least two clusters.

Vagotomy. Mice were anesthetized via an IP injection of a Ketamine (144 mg/kg)/Xylazine (13 mg/kg) cocktail. A midline incision was made and the stomach was retracted inferiorly to expose the distal esophagus and the gastroesophageal junction. The anterior (left) and posterior (right) branches of the vagus nerve were identified running alongside the esophagus and severed distal to the hepatic branches. The stomach was then placed back into the anatomical position and a pyloromyotomy was performed using a bent 23 gauge needle. The superficial muscular layers were incised in a longitudinal fashion and closed transversely with 4-0 vicryl sutures. The peritoneum was then closed with a running 4-0 vicryl suture and the skin approximated with staples. Animals were allowed to recover from anesthesia under a heat lamp and returned to the colony room once awake and ambulating. For non-vagotomized mice, the vagus nerve was gently exposed without further manipulation. Animals were monitored for 7 days. The completeness of subdiaphragmatic vagotomy was verified by examining fluorescent label of the dorsal motor vagal nucleus (DMV) on brainstem sections one week after intraperitoneal injection of FluoroGold. No presence of fluorescent label in DMV neurons was accepted as a marker of complete vagotomy.

Mass spectrometry. High resolution LC-MS analysis was performed on a Dionex 3000 UPLC coupled with a Thermo Q-exactive high-resolution mass spectrometer equipped with a HESI ion source. Metabolites were separated using a water-acetonitrile gradient on a Agilent Zorbax Eclipse XDB-C18 column (150 mm×2.1 mm, particle size 1.8 μm) maintained at 40° C.; solvent A: 0.1% formic acid in water; solvent B: 0.1% formic acid in acetonitrile. The AB gradient started at 1% B for 1 min after injection and increased linearly to 100% B at 15 min, using a flow rate of 0.5 mL/min. Mass spectrometer parameters: spray voltage 2.9 kV, capillary temperature 320° C., prober heater temperature 300° C.; sheath, auxiliary, and spare gas 70, 2, and 0 mL/min, respectively; S-lens RF level 55, resolution 140,000 at m/z 200, AGC target 1×10⁶. The instrument was calibrated weekly with positive and negative ion calibration solutions (Thermo-Fisher). Each sample was analyzed in negative and positive modes using a m/z range of 100 to 1500.

Feature detection, characterization and compound synthesis. LC-MS RAW files from triplicate fecal, serum and CSF samples from adult ex-GF fostered, Ctrl_fostered and GF mice were converted to mzXML (profile mode) using MSConvert (ProteoWizard), followed by analysis using a customized XCMS R-script based on the centWave XCMS algorithm to extract features. Resulting tables of all detected features were used to compute ex-GF fostered mice vs. GF mice and Ctrl_fostered mice vs. GF mice peak area ratios. To select differential features, we applied a filter retaining entries with peak area ratios larger than 2 (down in GF mice) or smaller than 0.5 (up in GF mice). We manually curated the resulting list to remove false positive entries, i.e., features that upon manual inspection of raw data were not differential. For the features that were verified to be differential, we examined elution profiles, isotope patterns, and MS1 spectra to find molecular ions and remove adducts, fragments, and isotope peaks.

The structures of the four differential compounds were confirmed by coinjection with synthesized or commercial samples. Phenyl sulfate and indoxyl sulfate were purchased from TCI America and Sigma-Aldrich, respectively. Pyrocatechol sulfate and 3-(3-sulfooxyphenyl)propanoic acid were prepared following a previously published procedure. To a stirred solution of catechol (Sigma-Aldrich, 0.55 g, 5 mmol) or 3-(4-hydroxyphenyl)propionic acid (Sigma-Aldrich, 0.88 g 5 mmol) in dry pyridine (2.5 mL), sulfur trioxide pyridine complex (0.88 g, 6 mmol) was added at room temperature. The resulting mixtures were heated in an oil bath at 45° C. and stirred for 2 hours. The reactions were then allowed to cool to ambient temperature and transferred separately to flasks each containing 25 mL of 1N KOH cooled in an ice bath. To each of the aqueous mixtures was added 100 mL of 2-propanol and the two reactions were left at 4° C. for 16 hours. At this point, the products were filtered off as white precipitates. The crude products were taken up in 50 mL (3:1 ethanol:water) and heated to reflux, hot filtered, and placed in the fridge for recrystallization. This last step was then repeated. Totals of 210 and 270 mg of pyrocatechol sulfate and 3-(3-sulfooxyphenyl)propanoic acid were obtained, corresponding to yields of ˜20%.

Statistical analysis. Statistical tests were performed with Prism (GraphPad). Unless specifically indicated otherwise, Student's t tests were used to compare end-point means of different groups. Error bars depict the SEM.

Data availability. RNA-seq data and 16S rRNA-seq data are available at Gene Expression Omnibus, BioProject under accession number GSE134808 and PRJNA556230, respectively. All datasets generated and/or analysed during the current study are presented in this published article, the accompanying Source Data or Supplementary Information, or are available from the corresponding author upon reasonable request.

### Example 2

We have identified a defect in levels of four microbiota-derived metabolites in the brain. These are phenyl sulfate, pyrocatechol sulfate, 3-(3-sulfooxyphenyl) propanoic acid, and indoxyl sulfate. Further, we identified that ex vivo exposure of brain-derived neurons a cocktail of these metabolites resulted in significant changes in neuronal development and activation.

To further evaluate the influence microbiota-derived metabolites on neuronal development and fear extinction learning, individual metabolites or cocktails of metabolites can be administered to either germ-free or ABX-treated wild-type mice prior to and during fear extinction learning. For example, groups of 8 mice can receive one of the following four metabolites: phenyl sulfate, pyrocatechol sulfate, 3-(3-sulfooxyphenyl) propanoic acid (all phenolic compounds) or indoxyl sulfate. Metabolites can be administered via intraperitoneal injection, oral delivery and intracranial injection. It can be tested whether delivery of metabolites in early-life (first two weeks post-birth) or delivery in adult mice (>8 weeks of age), has differential effects on neuronal activity or animal behavior. Restoration of metabolite levels in the circulation and brain that will result in restoration of fear extinction learning can be determined by delivery of individual metabolites or combinations of metabolites to microbiota-deficient animals.

In addition, based at least part on the teachings of the present disclosure, predictive analysis of microbiota-resident bacterial species that can express these metabolites, Clostridium and Bacteroides species (outlined above)) can be administered by oral gavage into either germ-free mice or ABX-treated mice. The consortium of bacteria can be administered either in early-life (first two weeks post-birth) or delivery in adult mice (>8 weeks of age), and it can be determined if such administration has differential effects on neuronal activity or animal behavior. Colonization of microbiota-deficient animals with this cocktail of bacteria should restore metabolite levels in the circulation and brain that will result in restoration of fear extinction learning and other neurological disorders.

### Example 3

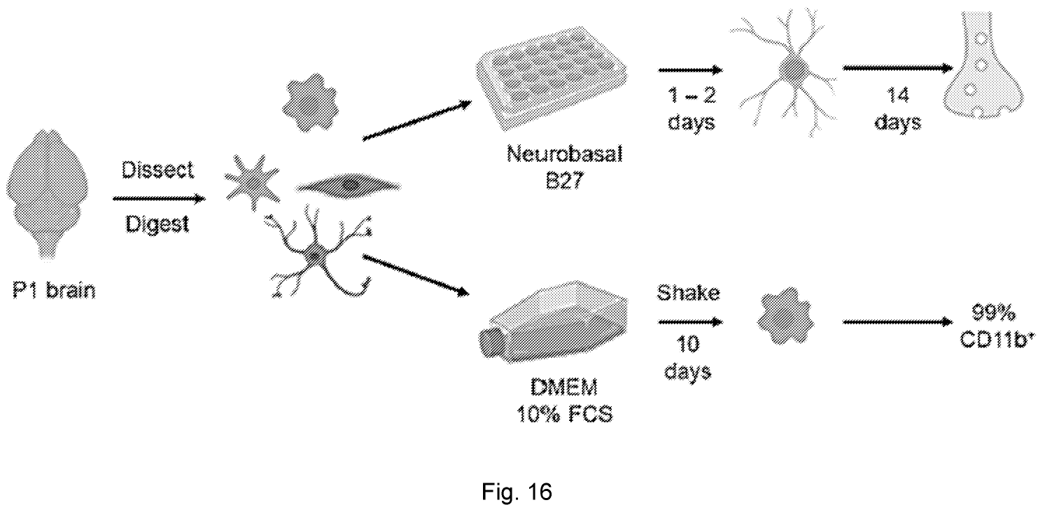

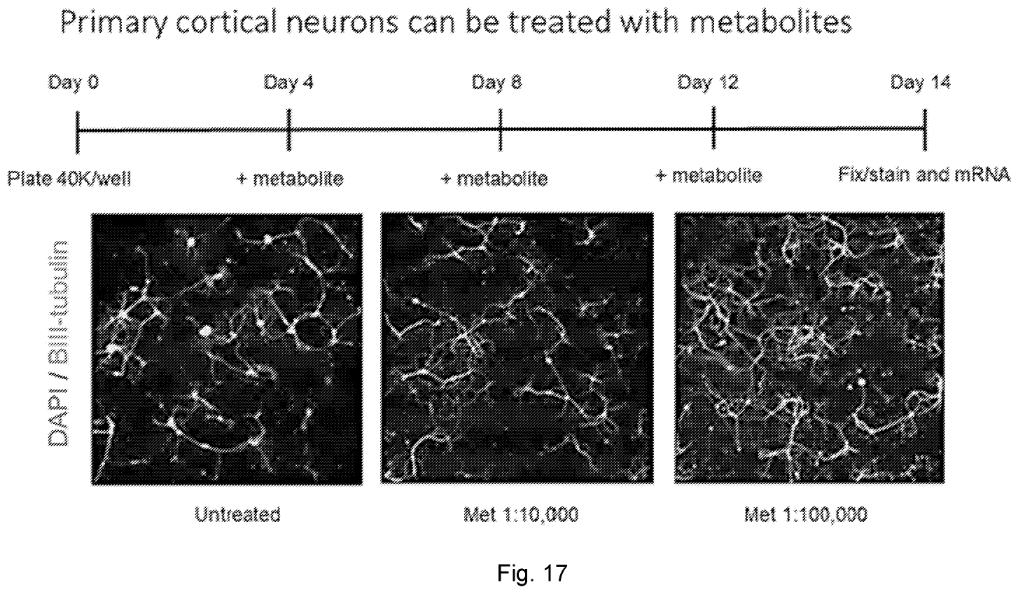

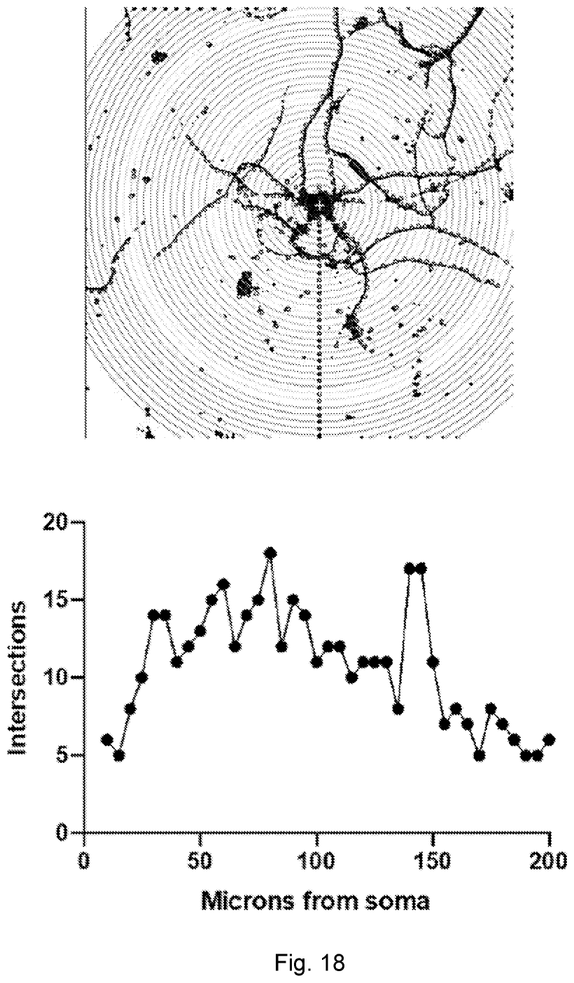

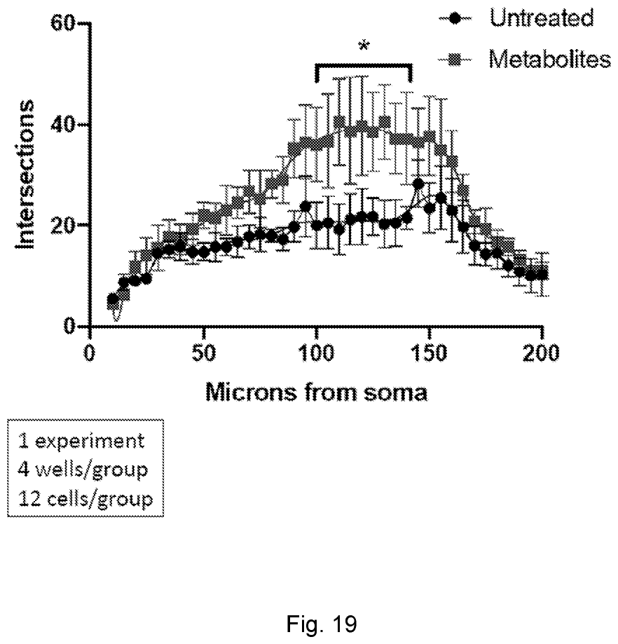

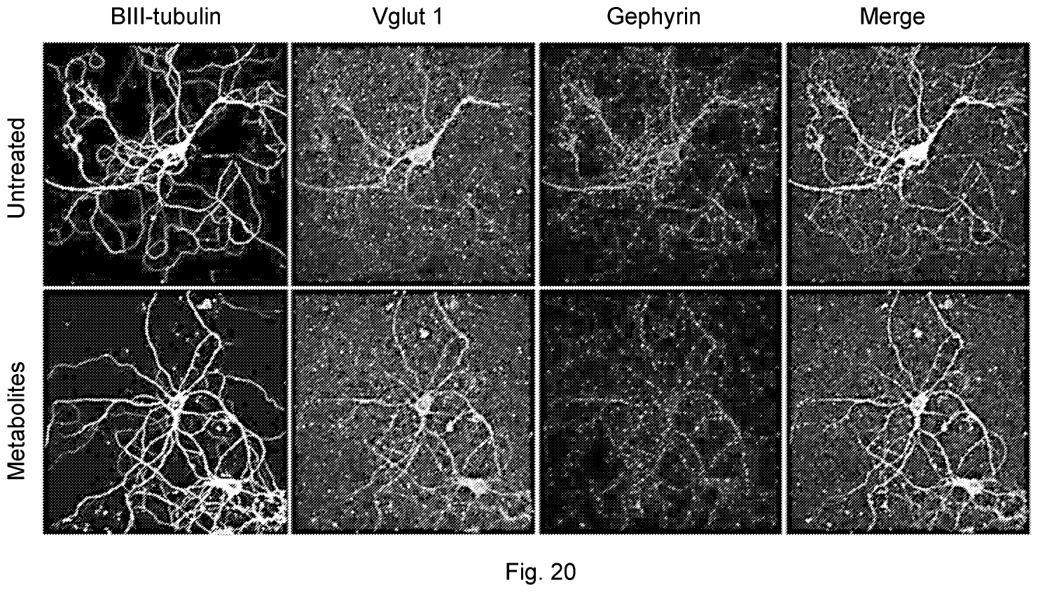

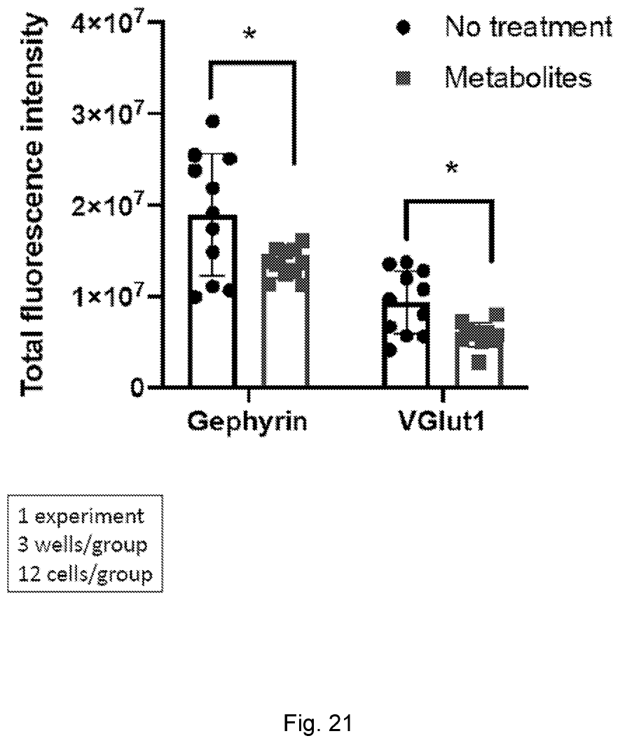

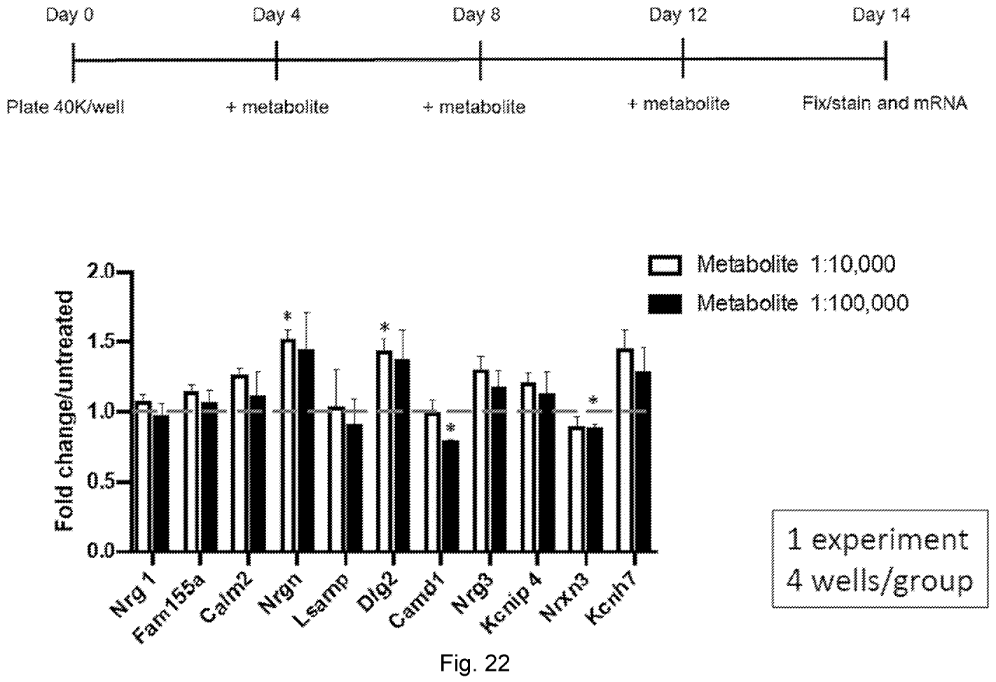

This example describes the effect of the metabolites on CNS cell activity or numbers. CNS cells were obtained by standard methods. A scheme for dissociation of CNS cultures is illustrated in FIG. 16. Primary cortical neurons were treated with metabolites or a cocktail (FIG. 17). A typical neuronal dendritic complexity is illustrated in FIG. 18A and FIG. 18B shows the intersections of the dendrites as a function of distance from the soma. The complexity of the dendritic interactions was observed to increase upon incubation with the metabolites (FIG. 19). Incubation with metabolites altered of excitatory versus inhibitory neurons (FIG. 20) and alters excitatory vs inhibitory balance (FIG. 21). Treatment of neuronal cultures with metabolites alters multiple RNA species as shown in FIG. 22.

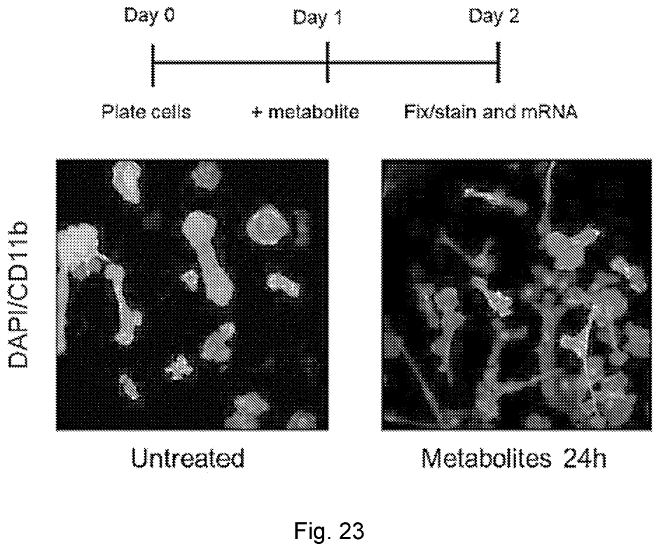

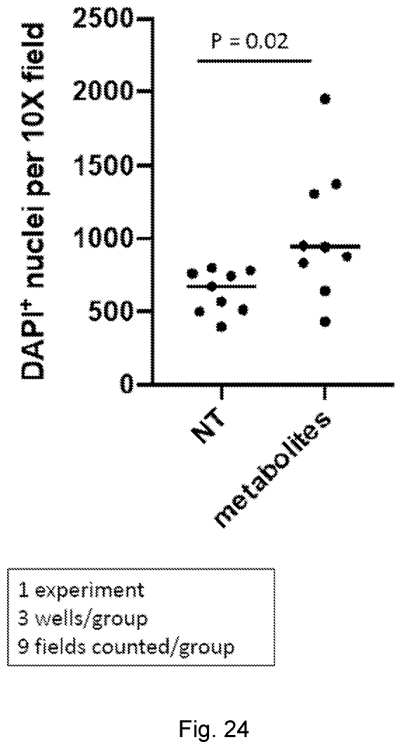

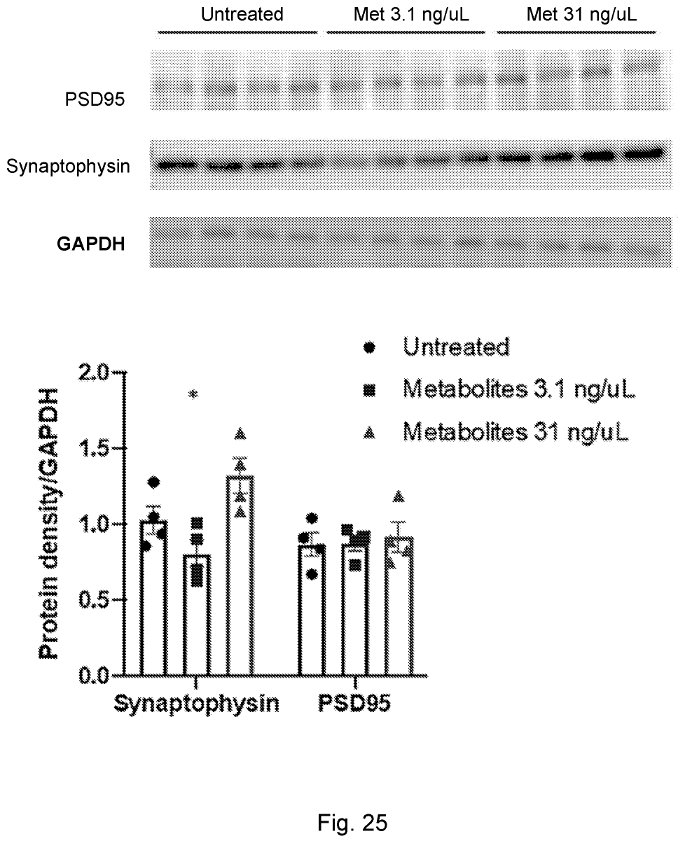

Effects of metabolites on other cell types were also seen. FIGS. 23 and 24 shows that: Metabolite treatment induces microglial proliferation. Further, FIG. 25 shows that metabolite treatment increases presynaptic protein expression in cultured neurons.

A predictive phylogenetic analysis of bacteria indicated which bacteria could be sources of one or more of phenyl sulfate, pyrocatechol sulfate, 3-(3-sulfooxyphenyl)propanoic acid, and indoxyl sulfate.

### Example 4

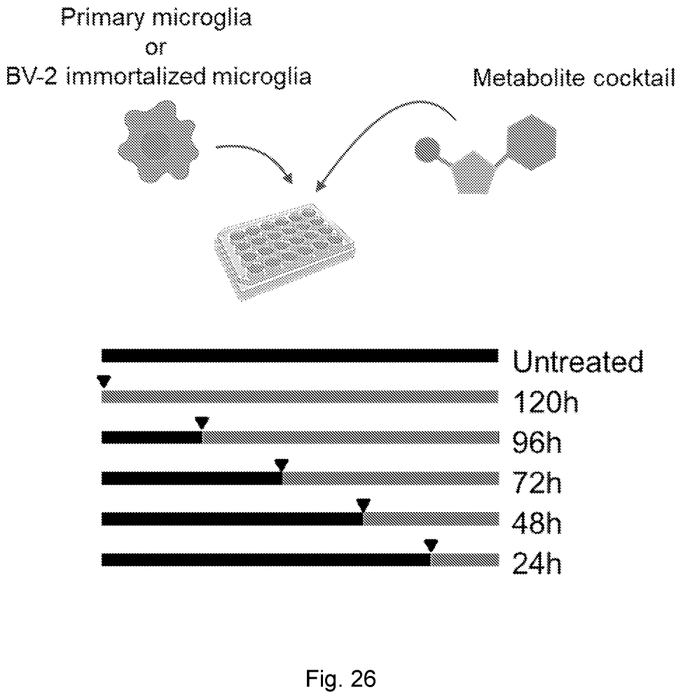

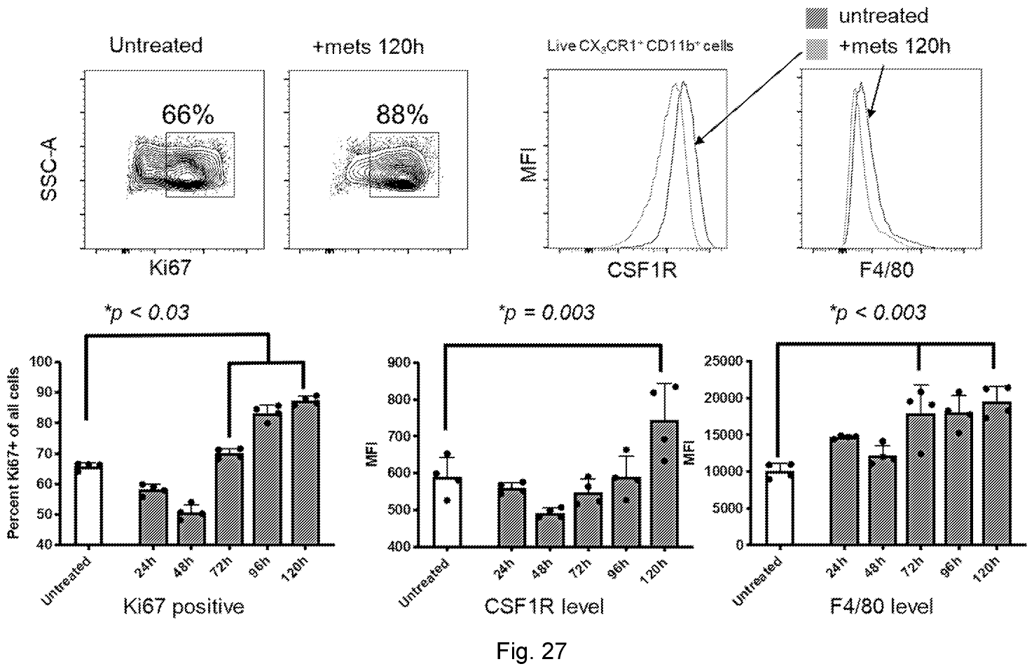

Purified cultures, or immortalized equivalents were used. The cells were treated with a cocktail of the 4 metabolites to evaluate microglial response to metabolite treatment (FIG. 26). We found that in BV-2 microglial cells our cocktail of metabolites is highly biologically active in terms both inducing proliferation (percent Ki67+ cells at 120 h of metabolite treatment upper left and quantified lower left) and cell surface protein expression (CSF1R and F4/80 MFI upper right, quant lower right) (FIG. 27). There is a relatively linear effect with increasing proportions of proliferation or F4/80, CSF1R high cells with increasing exposure to the metabolite cocktail.

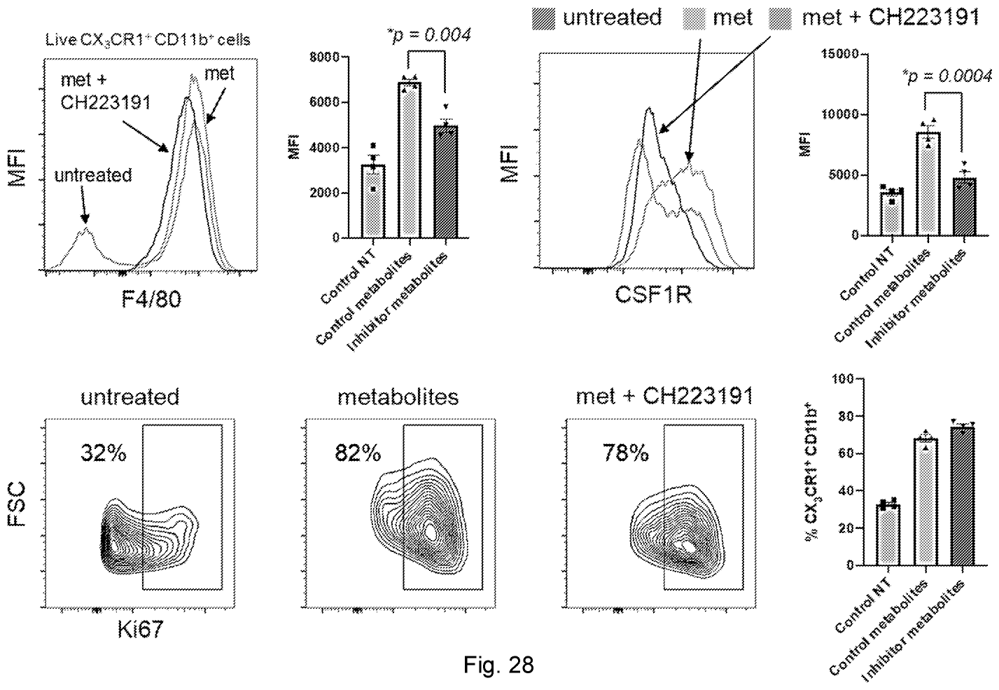

Next, we tested whether intact AhR signaling was required for these effects by blocking AhR signaling with the validated small molecular inhibitor CH223191 and found that it is required for the upregulation of CSF1R and F4/80, but dispensable for enhancement of proliferation (FIG. 28).

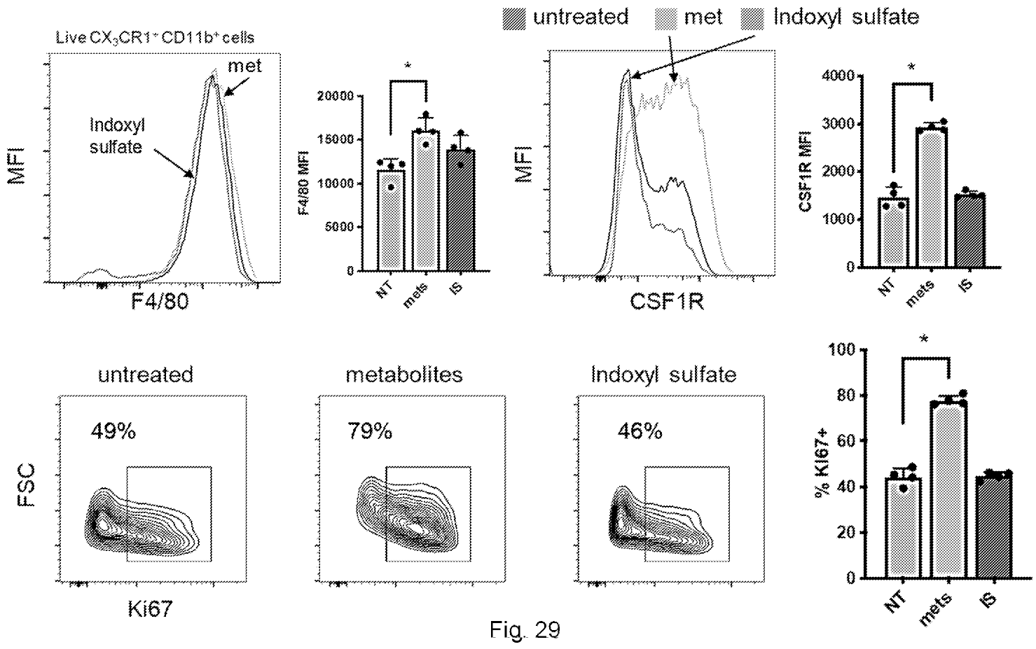

We next asked if individual metabolites were responsible for the biological activity of the metabolite cocktail or whether multiple metabolites were required. We started with indoxyl sulfate given that it is a known ligand of AhR and has been implicated in a number of neuropsychiatric disease states and found that IS alone is unable to significantly change either cell surface levels of F4/80 or CSF1R or to increase proliferation (FIG. 29).

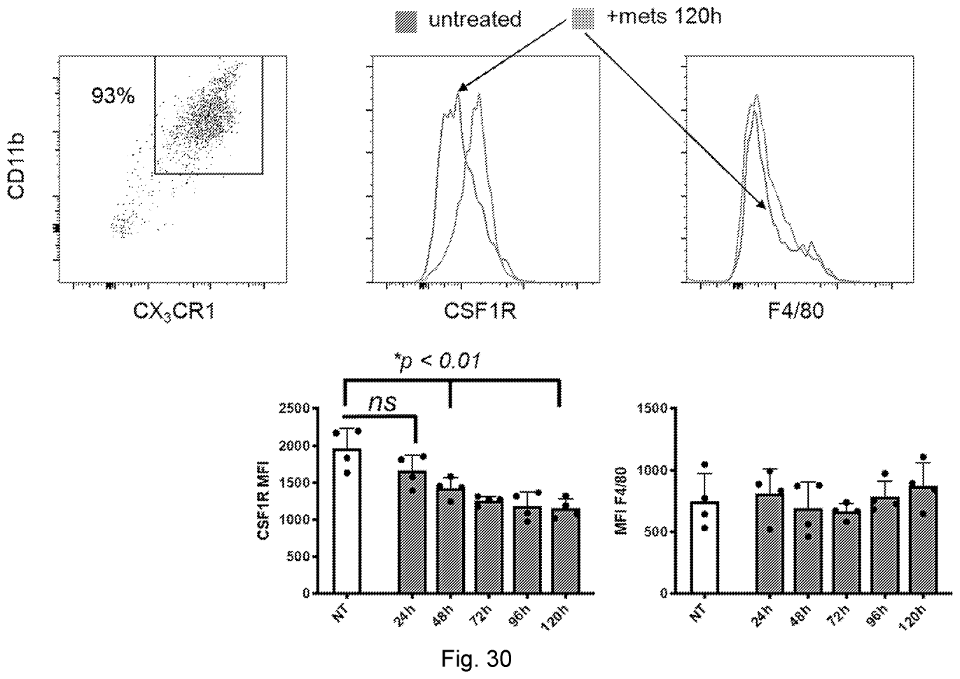

In order to verify that metabolites have similar activities in primary CNS cells we performed similar experiments in primary microglia purified from P1 SPF mice and found that the cocktail of 4 metabolites altered CSF1R surface levels (FIG. 30).

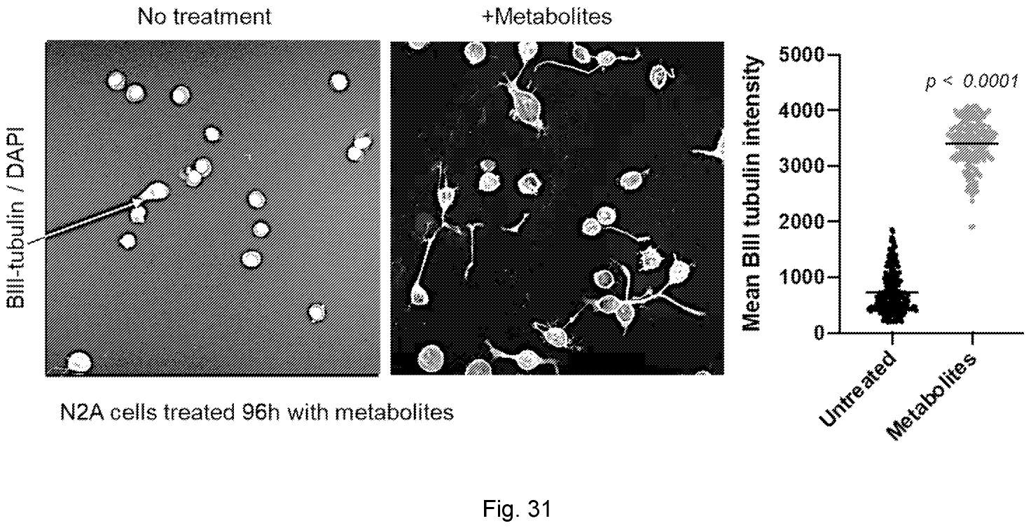

We also tested the cocktail of metabolites on the immortalized neuronal cell line Neuro2A and stained for the critical neuronal structural protein Beta-III-tubulin and found that treatment of N2A cells for 96 hours significantly increased the level of BIII-tubulin (FIG. 31).

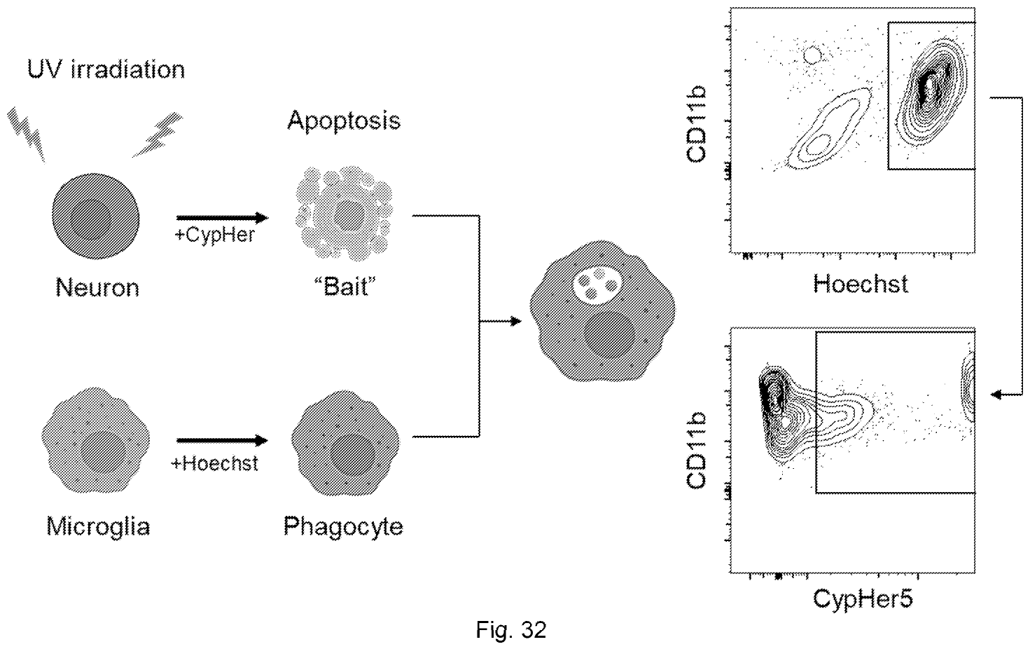

Finally, we asked whether treatment with metabolites altered the ability of BV-2 microglia to engulf apoptotic N2A cells as outlined above. In this assay the phagocytes (in this case BV-2 cells) are treated with metabolites or vehicle for 120 h and then loaded with Hoechest dye. N2A cells are killed with UV radiation (confirmed by staining with viability dyes and Annexin V to distinguish apoptotic from necrotic cells) and then loaded with the pH sensitive dye CypHer5 which only fluoresces in the phagolysosome. They are then fed to BV-2 cells for a predetermined length of time (5:1 ratio of N2A to BV-2, 1 h of eating) before being harvested and analyzed by flow cytometry (FIG. 32).

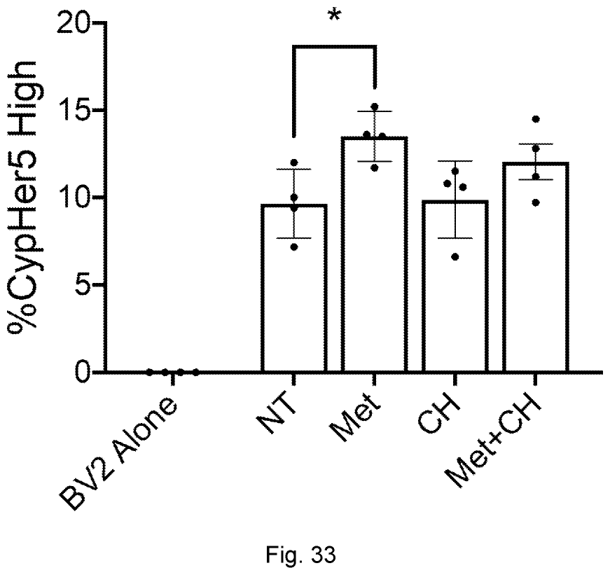

Next we observed that compared to BV-2 cells alone, BV-2 cells treated with metabolites (met) show a significantly higher degree of phagocytosis when compared to BV-2 cells treated with vehicle alone (NT). This effect is NOT dependent on AhR signaling (Met+CH) (FIG. 33).

### Example 5

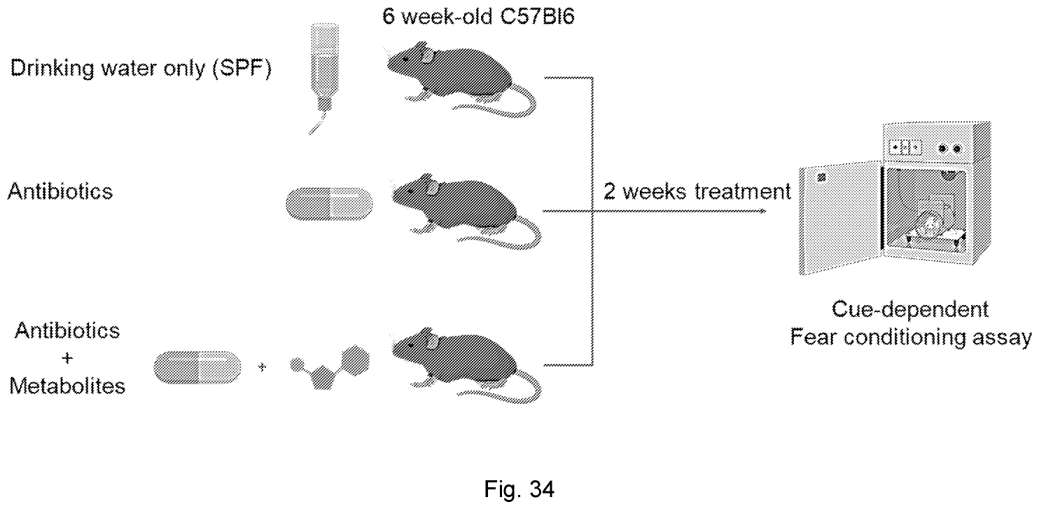

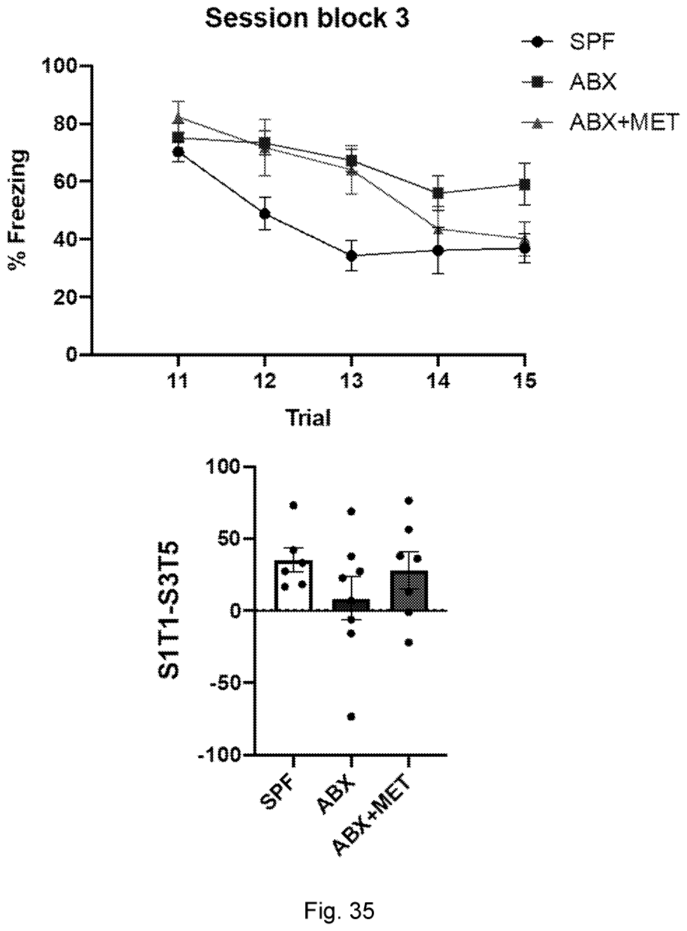

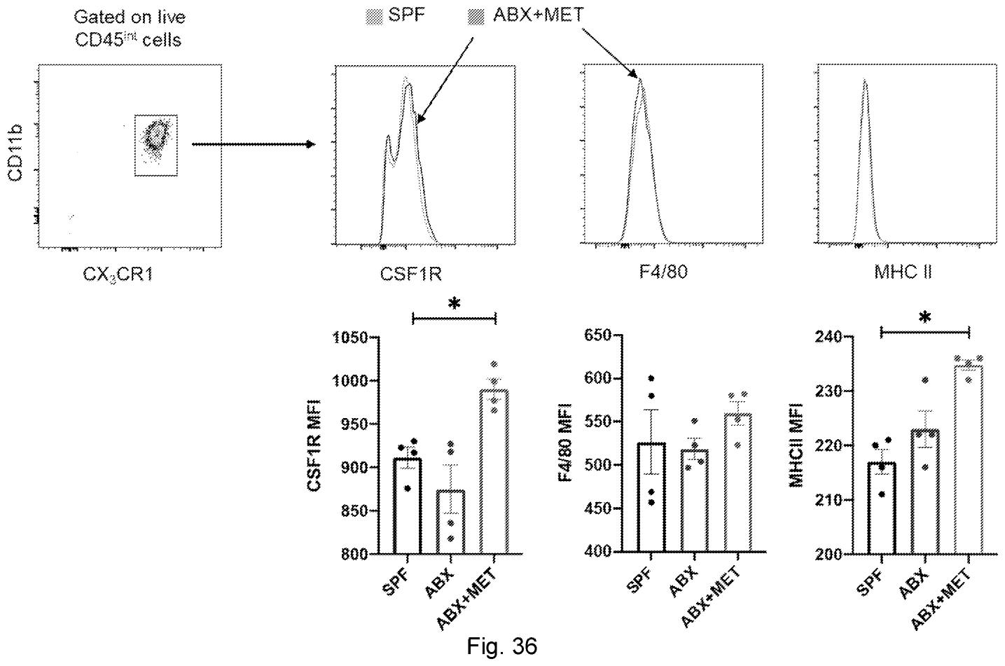

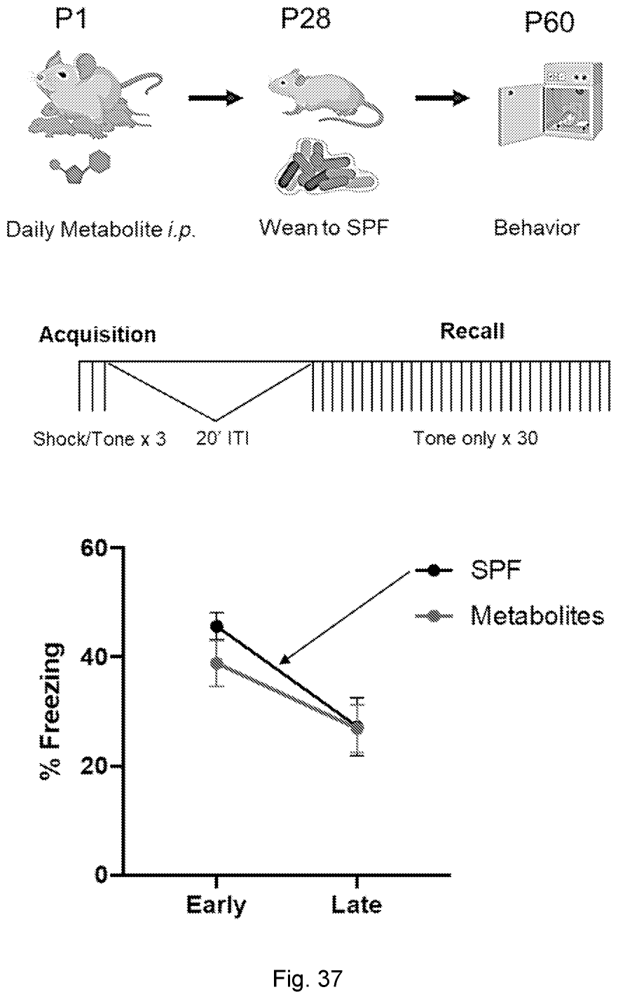

This example describes in vivo data. FIG. 34 shows experimental outline for the acute depletion of microbiota in adult animals. 6 week-old male C57Bl6 mice were treated with drinking water alone (control SPF group), antibiotics (neo, vanc, metronidazole, ampicillin, gent) in drinking water, or antibiotics plus a cocktail of the metabolites at a concentration of 32 ug/mL for a total of 2 weeks. The mice were then subjected to cue-dependent tone-shock fear conditioning. The FC assay consists of a single day conditioning regimen (3 tone/shock pairings, 0.5 mA shock) followed 3 recall blocks (5 trials per day ×3 days total). FIG. 35 shows that treatment with the metabolite cocktail rescues fear conditioning in antibiotic-treated mice. Results of The left hand figure shows the percent of time freezing during each trial within the 3^{rd} session block, the right hand figure shows the differential freezing (session 1, trial 1-session 3, trial 5). N=6-8 mice per group. Error is shown as SEM. Similar results are seen in germ-free mice. FIG. 36 shows that metabolite treatment alters the cell surface profile of microglia in vivo. FIG. 37 depicts the experimental setup and results of an experiment designed to test the capacity of a cocktail of metabolites to rescue defects in fear conditioning in germ-free mice. In this experiment, GF mice were given a cocktail of metabolites at 1.6 ug/g of body weight by daily i.p. injection. They were then weaned into an SPF environment (dirty SPF bedding swap) at P28 and then tested in a modified one day tone/shock cue-dependent fear conditioning assay as depicted. The figure on the right represents data from 8 mice per group with error as SEM.

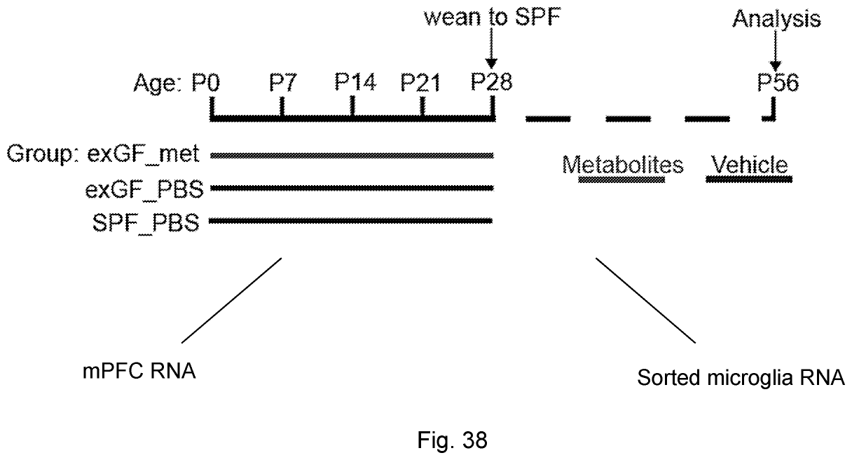

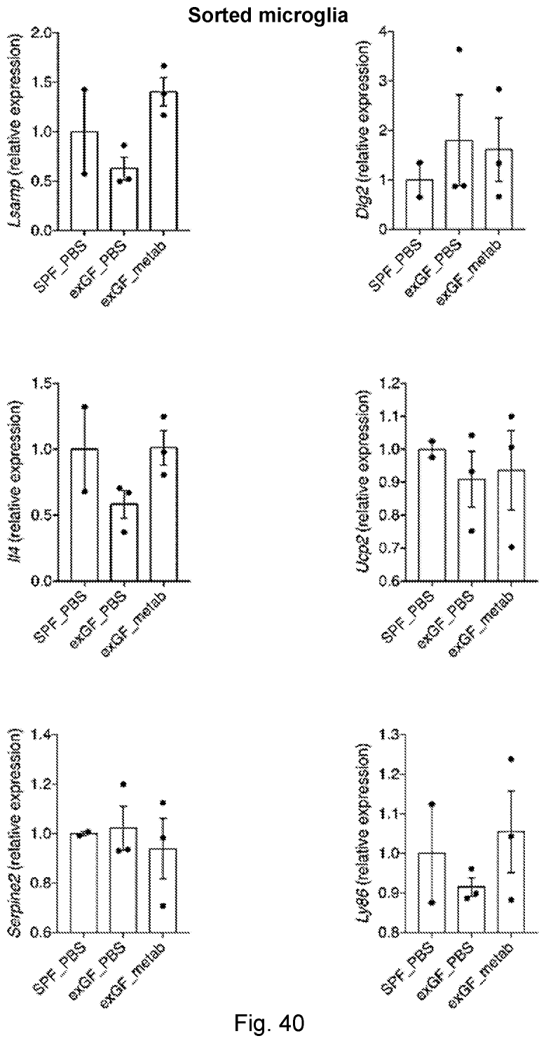

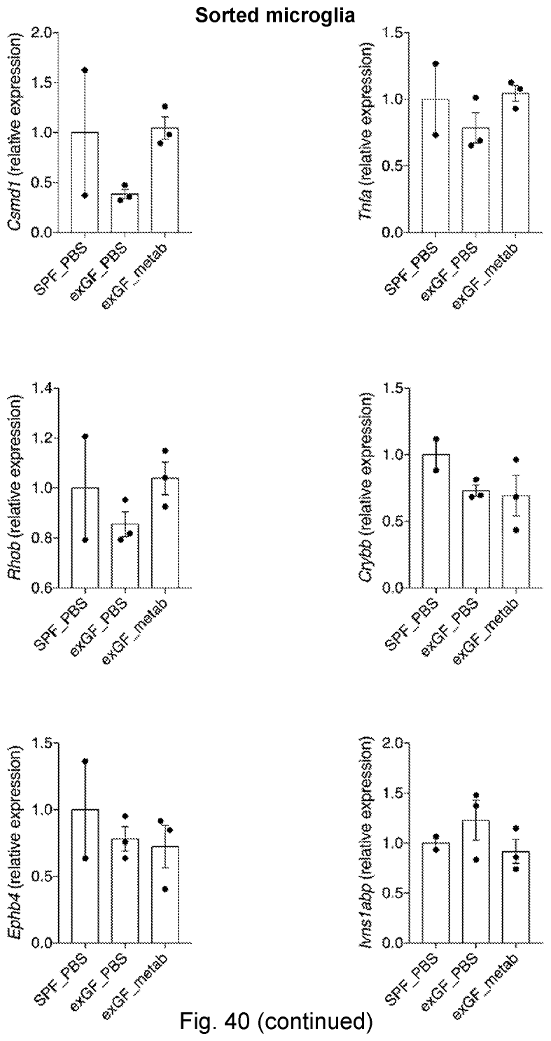

Rescue experiments were carried out using metabolite cocktail in cultured microglia. Results are shown in FIG. 38. This slide depicts the experiments design for a separate set of experiments. Here germ-free mice were given either PBS or 1.6 ug/g of body weight metabolite cocktail i.p. every 3 days starting at P1 and continuing until weaning. Mice were then switched to SPF housing conditions. At 6 weeks of age groups of mice were sacrificed and either the entire mPFC was collected and bulk RNA was extracted and underwent qPCR for the genes in the following slide (normalized to Hprt) OR microglia were sort purified from the whole brain and subjected to the same Hprt-normalized qPCR in FIG. 40.

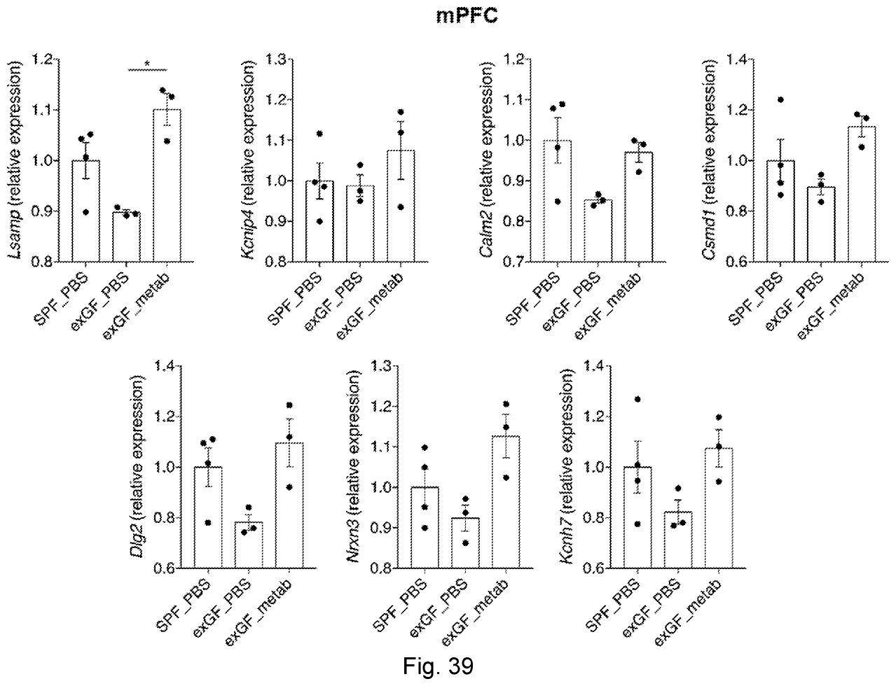

Relative expression of several genes was measured in mPFC and microglia cells was evaluated in control (SPF), treated with PBS or with metabolite cocktail (FIG. 39 and FIG. 40).

Although the present disclosure has been described in detail for the purpose of illustration, it is understood that such detail is solely for that purpose and variations can be made by those skilled in the art without departing from the spirit and scope of the disclosure which is defined by the following claims.

1. A pharmaceutical composition comprising at least two of phenyl sulfate, pyrocatechol sulfate, 3-(3-sulfooxyphenyl)propanoic acid, and indoxyl sulfate.

2. The pharmaceutical composition of claim 1 comprising at least three of phenyl sulfate, pyrocatechol sulfate, 3-(3-sulfooxyphenyl)propanoic acid, and indoxyl sulfate.

3. The composition of claim 1 comprising phenyl sulfate, pyrocatechol sulfate, 3-(3-sulfooxyphenyl)propanoic acid, and indoxyl sulfate.

4. The composition of claim 1, further comprising non-pathogenic bacteria from the Clostridium and/or Bacteroides species.

5. The composition of claim 1 formulated for oral administration.

6. A pharmaceutical composition comprising bacteria that produce phenyl sulfate, pyrocatechol sulfate, 3-(3-sulfooxyphenyl)propanoic acid, and indoxyl sulfate.

7. The pharmaceutical composition of claim 6, wherein the bacteria that produce phenyl sulfate, pyrocatechol sulfate, 3-(3-sulfooxyphenyl)propanoic acid, and indoxyl sulfate are the only bacteria present in the composition.

8. The pharmaceutical composition of claim 7, wherein the composition is present in a dried form.

9. The pharmaceutical composition of claim 7, wherein the bacteria are from the Clostridium and/or Bacteroides species.

10. A method of treating a neurological or behavioral disorder by administration to an individual diagnosed with the neurological or behavioral disorder, a composition comprising two or more of phenyl sulfate, pyrocatechol sulfate, 3-(3-sulfooxyphenyl)propanoic acid, and indoxyl sulfate.

11. The method of claim 10, wherein the individual is administered phenyl sulfate, pyrocatechol sulfate, 3-(3-sulfooxyphenyl)propanoic acid, and indoxyl sulfate.

12. The method of claim 11, wherein the circulating levels in the individual of phenyl sulfate, pyrocatechol sulfate, 3-(3-sulfooxyphenyl)propanoic acid, and indoxyl sulfate increase to normal physiologic levels upon administration of a composition comprising phenyl sulfate, pyrocatechol sulfate, 3-(3-sulfooxyphenyl)propanoic acid, and indoxyl sulfate.

13. The method of claim 10, wherein the behavioral disorder is anxiety disorder.

14. The method of claim 13, wherein the anxiety disorder is associated with a deficit in extinction learning.

15. The method of claim 10, further comprising administering to the individual bacteria that increases the amounts of one or more of the following in the individual: phenyl sulfate, pyrocatechol sulfate, 3-(3-sulfooxyphenyl)propanoic acid, and indoxyl sulfate.

16. The method of claim 15, wherein the levels of said compounds are increased in serum, cerebrospinal fluid, or fecal matter excreted by said animal.

17. The method of claim 15, wherein the bacteria are non-pathogenic bacteria from the Clostridium and/or Bacteroides species.

18. The method of claim 10, wherein the individual has panic disorder or post traumatic stress disorder.

19. A method of increasing the number, function or activity of microglia or neurons in mammalian nervous system comprising administering to an individual in need of treatment, a composition comprising two or more of phenyl sulfate, pyrocatechol sulfate, 3-(3-sulfooxyphenyl)propanoic acid, and indoxyl sulfate, and optionally, bacteria that result in increasing the concentration of phenyl sulfate, pyrocatechol sulfate, 3-(3-sulfooxyphenyl)propanoic acid, and indoxyl sulfate in the individual.

20. The method of claim 19, wherein the bacteria are non-pathogenic bacteria from the Clostridium and/or Bacteroides species.


SEQUENCE LISTING (2 sequences)

SEQ ID NO: 1 - DNA, 21 bp, Artificial Sequence
  primer
actcctacgggaggcagcagt

SEQ ID NO: 2 - DNA, 18 bp, Artificial Sequence
  primer
attaccgcggctgctggc